SEQUENCE LISTING (37 sequences)

SEQ ID NO: 1 - DNA, 9810 bp, Artificial
  Artificial Sequence
gcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttgatccggaattaattcctgtggttggcatgcacatacaaatggacgaacggataaaccttttcacgcccttttaaatatccgattattctaataaacgctcttttctcttaggtttacccgccaatatatcctgtcaaacactgatagtttaaactgaaggcgggaaacgacaatctgatcatgagcggagaattaagggagtcacgttatgacccccgccgatgacgcgggacaagccgttttacgtttggaactgacagaaccgcaacgctgcaggaattggccgcagcggccatttaaatcaattgggcgcgccgaattcgagctcggtaccgggccccccctcgaggccgaccaaccgcaagcgttgtcagtgttgcaaagcgctctgtgtgggcctactttaattgcttccagtgttaaattggcgaaaggcaataatatcgcaaaatattgtgttgtaaaatgtaattatgttttaatttcatggaaatgtttgagcataatttttattaatgtactaaattactgttttgttaaatgcaattttgctttctcgggattttaatatcaaaatctatttagaaatacacaatattttgttgcaggcttgctggagaatcgatctgctatcataaaaattacaaaaaaattttatttgcctcaattattttaggattggtattaaggacgcttaaattatttgtcgggtcactacgcatcattgtgattgagaagatcagcgatacgaaatattcgtagtactatcgataatttatttgaaaattcataagaaaagcaaacgttacatgaattgatgaaacaatacaaagacagataaagccacgcacatttaggatattggccgagattactgaatattgagtaagatcacggaatttctgacaggagcatgtcttcaattcagcccaaatggcagttgaaatactcaaaccgccccatatgcaggagcggatcattcattgtttgtttggttgcctttgccaacatgggagtccaaggtttcagggaagctggaattcactagtgattgtaaaacgacggccagtgccttttttttttttttttttttacactcaagatctcgatgcaattctcacacgaataaggtacaaagttcatcaaccttttgtcttaaaacagatagtattgacttagttccgtctacttaagtatcacacacaaagtctcttggccaaagagagagtattaagatatacatacgctcgcccttatcagcggaataattacatatcttatttttttttcttcataattatatatgttttggattttgatttcaaccgccgatttaaggtggttaattaagtagtgggagagtcaccggaagattgtcggagatttgtccagcaggtgacatctccatctcagcttctgctcccacatgatgattattctccatctggctagccaaaacctggttctcttctttcagcattttctccttttctttaagattcacaacaagcttcaacatgagttcggtcttcttggctctagtcacggagagggcagtctcaaggtgttcctccagttgaacaagagcatcgatactcacatttttgacatttgatcccacaagcttgctatccacaagttcaagtagctcatagtgtgaaccatagttcagagcttttgactgatgatccaaggctttaagatcatcagcatgctgtttcccatatcgatcaaggatcttgaccaggttatcgccggaggagaagctgtagagcttgccggaggcggagacgacgagaagagcgacggatgcgtcacagagaacagaaagctgacgagctttctcgatgagaccgttgcgacgtttggagaaggtgacttgtcggctacttttgttctcaattcgcttgatttctaatttctttcttcccatggtcaagagtcccccgtgttctctccaaatgaaatgaacttccttatatagaggaagggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcagaggcatcttcaacgatggcctttcctttatcgcaatgatggcatttgtaggagccaccttccttttccactatcttcacaataaagtgacagatagctgggcaatggaatccgaggaggtttccggatattaccctttgttgaaaagtctcaattgccctttggtcttctgagactgtatctttgatatttttggagtagacaagtgtgtcgtgctccaccatgttgacgaagattttcttcttgtcattgagtcgtaagagactctgtatgaactgttcgccagtctttacggcgagttctgttaggtcctctatttgaatctttgactccatgaagctaaactgaaggcgggaaacgacaatctgatccaagctcaagctgctctagcattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagcttgcatgcctgcaggcatgcaagcttcgtacgttaattaattcgaatccggagcggccgcacgcgtgggcccgtttaaacctcgagagatctgctagcatcgatggtaccgagctcgagactagctacaggccaaattcgctcttagccgtacaatattactcaccggtgcgatgccccccatcgtaggtgaaggtggaaattaatgatccatcttgagaccacaggcccacaacagctaccagtttcctcaagggtccaccaaaaacgtaagcgcttacgtacatggtcgataagaaaaggcaatttgtagatgttaacatccaacgtcgctttcagggatcccgaattccaagcttggaattcgggatcctacaggccaaattcgctcttagccgtacaatattactcaccggtgcgatgccccccatcgtaggtgaaggtggaaattaatgatccatcttgagaccacaggcccacaacagctaccagtttcctcaagggtccaccaaaaacgtaagcgcttacgtacatggtcgataagaaaaggcaatttgtagatgttaacatccaacgtcgctttcagggatcccgaattccaagcttggaattcgggatcctacaggccaaattcgctcttagccgtacaatattactcaccggtgcgatccccccatcgtaggtgaaggtggaaattaatgatccatcttgagaccacaggcccacaacagctaccagtttcctcaagggtccaccaaaaacgtaagcgcttacgtacatggtcgataagaaaaggcaatttgtagatgttaacatccaacgtcgctttcagggatcccgaattccaagcttgggctgcaggtcaatcccattgcttttgaagcagctcaacattgatctctttctcgagggagatttttcaaatcagtgcgcaagacgtgacgtaagtatccgagtcagtttttatttttctactaatttggtcgtttatttcggcgtgtaggacatggcaaccgggcctgaatttcgcgggtattctgtttctattccaactttttcttgatccgcagccattaacgacttttgaatagatacgctgacacgccaagcctcgctagtcaaaagtgtaccaaacaacgctttacagcaagaacggaatgcgcgtgacgctcgcggtgacgccatttcgccttttcagaaatggataaatagccttgcttcctattatatcttcccaaattaccaatacattacactagcatctgaatttcataaccaatctcgatacaccaaatcgagatctgcagggatccccgatcatgcaaaaactcattaactcagtgcaaaactatgcctggggcagcaaaacggcgttgactgaactttatggtatggaaaatccgtccagccagccgatggccgagctgtggatgggcgcacatccgaaaagcagttcacgagtgcagaatgccgccggagatatcgtttcactgcgtgatgtgattgagagtgataaatcgactctgctcggagaggccgttgccaaacgctttggcgaactgcctttcctgttcaaagtattatgcgcagcacagccactctccattcaggttcatccaaacaaacacaattctgaaatcggttttgccaaagaaaatgccgcaggtatcccgatggatgccgccgagcgtaactataaagatcctaaccacaagccggagctggtttttgcgctgacgcctttccttgcgatgaacgcgtttcgtgaattttccgagattgtctccctactccagccggtcgcaggtgcacatccggcgattgctcactttttacaacagcctgatgccgaacgtttaagcgaactgttcgccagcctgttgaatatgcagggtgaagaaaaatcccgcgcgctggcgattttaaaatcggccctcgatagccagcagggtgaaccgtggcaaacgattcgtttaatttctgaattttacccggaagacagcggtctgttctccccgctattgctgaatgtggtgaaattgaaccctggcgaagcgatgttcctgttcgctgaaacaccgcacgcttacctgcaaggcgtggcgctggaagtgatggcaaactccgataacgtgctgcgtgcgggtctgacgcctaaatacattgatattccggaactggttgccaatgtgaaattcgaagccaaaccggctaaccagttgttgacccagccggtgaaacaaggtgcagaactggacttcccgattccagtggatgattttgccttctcgctgcatgaccttagtgataaagaaaccaccattagccagcagagtgccgccattttgttctgcgtcgaaggcgatgcaacgttgtggaaaggttctcagcagttacagcttaaaccgggtgaatcagcgtttattgccgccaacgaatcaccggtgactgtcaaaggccacggccgtttagcgcgtgtttacaacaagctgtaagagcttactgaaaaaattaacatctcttgctaagctgggagctcgtcgacggatcgaattcctgcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaacctaggataaattatcgcgcgcggtgtcatctatgttactagatctctagaactagtggatctgctagccctgcaggaaatttaccggtgcccgggcggccagcatggccgtatccgcaatgtgttattaagttgtctaagcgtcaatttgtttacaccacaatatatcctgccaccagccagccaacagctccccgaccggcagctcggcacaaaatcaccactcgatacaggcagcccatcagaattaattctcatgtttgacagcttatcatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatccggctcgtataatgtgtggaattgtgagcggataacaatttcacacaggaaacagaccatgagggaagcgttgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctaggcaggcttatcttggacaagaagatcgcttggcctcgcgcgcagatcagttggaagaatttgttcactacgtgaaaggcgagatcaccaaagtagtcggcaaataaagctctagtggatccccgaggaatcggcgtgagcggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagctctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgt

SEQ ID NO: 2 - DNA, 11051 bp, Artificial
  Artificial
gtttacccgccaatatatcctgtcaaacactgatagtttaaactgaaggcgggaaacgacaatctgatcatgagcggagaattaagggagtcacgttatgacccccgccgatgacgcgggacaagccgttttacgtttggaactgacagaaccgcaacgctgcaggaattggccgcagcggccatttaaatcaattgggcgcgccgaattcgagctcggtacccggggatcctctagatcatgtttgacagcttatcatcggatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattagatgataatcatcgcaagaccggcaacaggattcaatcttaagaaactttattgccaaatgtttgaacgatctctgcaggtcgacgcatgctgatctcctgatcatctgctcgttgttggcgacgtagcagatctggtgaagaattgagaaccttatctttcaaccggacattttctttgattaagtgcttctcctcttcatacaacttattaatctgctctttgtacaatgcattctttcttgctcggatactggaaagacttttatctaattgtttttctaattcttgaagctcatcaatggaacatgcctctagaccatctccaagtaatttccgtttagaaagttcgagactttttaacttcttccgcggacctgcacatcaacaaattttggtcatatattagaaaagttataaattaaaatatacacacttataaactacagaaaagcaattgctatatactacattcttttattttgaaaaaaatatttgaaatattatattactactaattaatgataattattatatatatatcaaaggtagaagcagaaacttacctaagaagttaaaaagtctcgaactttctaaacggaaattacttggagatggtctagaggcatgttccattgatgagcttcaagaattagaaaaacaattagataaaagtctttccagtatccgagcaagaaagaatgcattgtacaaagagcagattaataagttgtatgaagaggagaagcacttaatcaaagaaaatgtccggttgaaagataaggttctcaattcttcaccagatctgctacgtcgccaacaacgagcagatgatcaggagatcagcatgcggatccaaagagagagtcgcgagagatttgcagagatcgctttaggctttgggagagattgaagagtcagaaaaagacgaaaggatgaattattatcttccacacgaaggtcttctttatatcgcaaaccaaaagcccaaaaccgtcttttctattaatgagaataaaatatctttagccaaaacaaaaaaaggaagatatcagttgaggattattatcacgaaactaaaggaaggaatcatatgatacgtgtcatattttccaccgtgcgtttttaaaagaccgactcaagtagagacatcctatggtggtggttggattaggtcatccattacatctgcttcactgacatttttctatttttctttttgtatatacttttcctcaaataatttctttcttttctatagaagaatttaatcaataaggaaaaagttcaaaaaagattctttccattaagactatgtcttggttaacccaacccattaagaataagcaatcataatatatatagagaatactaatactatatatgagatttttcttttaatttcatgttgattatgatagtttatcttcttgatttaatttatcaatacttggcataaaagattctaatctactctaataaagaaaagaaaaaaaagtatctaccattgactaattaaaataaggaaacttatctaccaaatttgagtattttttagaacaatctttttggtttaattccaaaactctaaacctaattgttgggaaaaaggacctaatttttaagaaaagttaataattagaagatctgtatgttttttttttgatccaagtttttatttcttttctctttttttcatgataaaatctatgtttttttagtctacaattaaagtaattgttattattttctttatctttttttgttgttgttgttaattccctttttttttttttaacagcaacttcttaaaaaaaaaaacagttgggccttgaatttatttcaggcctgcgttattaagcccagataataactcaaaacaaaaaaaatgttgaaccggaataaacccgcgagattaaatgccggttttcaggtaacatagaagaagaatatatgaggattgaagaagtattcaagaggcggaacaattcacaagtccaagagcttaaatttctcctcactcttctgctacagactcggaactctttctctttgctaaaataagatgttcaggatttttgttgcccgacaattcatgtatctcacactctctctcttctctgttcttactactctgttacattaccaccaactcaagactttcttccacaatggcgtttatgagacttggctccaaatccggacggatctctagagtcgaccatggtgatcactgcaggcatgcaagcttcgtacgttaattaattcgaatccggagcggccgcacgcgtgggcccgtttaaacctcgagagatctgctagcatcgatggtaccgagctcgagactagctacaggccaaattcgctcttagccgtacaatattactcaccggtgcgatgccccccatcgtaggtgaaggtggaaattaatgatccatcttgagaccacaggcccacaacagctaccagtttcctcaagggtccaccaaaaacgtaagcgcttacgtacatggtcgataagaaaaggcaatttgtagatgttaacatccaacgtcgctttcagggatcccgaattccaagcttggaattcgggatcctacaggccaaattcgctcttagccgtacaatattactcaccggtgcgatgccccccatcgtaggtgaaggtggaaattaatgatccatcttgagaccacaggcccacaacagctaccagtttcctcaagggtccaccaaaaacgtaagcgcttacgtacatggtcgataagaaaaggcaatttgtagatgttaacatccaacgtcgctttcagggatcccgaattccaagcttggaattcgggatcctacaggccaaattcgctcttagccgtacaatattactcaccggtgcgatccccccatcgtaggtgaaggtggaaattaatgatccatcttgagaccacaggcccacaacagctaccagtttcctcaagggtccaccaaaaacgtaagcgcttacgtacatggtcgataagaaaaggcaatttgtagatgttaacatccaacgtcgctttcagggatcccgaattccaagcttgggctgcaggtcaatcccattgcttttgaagcagctcaacattgatctctttctcgagggagatttttcaaatcagtgcgcaagacgtgacgtaagtatccgagtcagtttttatttttctactaatttggtcgtttatttcggcgtgtaggacatggcaaccgggcctgaatttcgcgggtattctgtttctattccaactttttcttgatccgcagccattaacgacttttgaatagatacgctgacacgccaagcctcgctagtcaaaagtgtaccaaacaacgctttacagcaagaacggaatgcgcgtgacgctcgcggtgacgccatttcgccttttcagaaatggataaatagccttgcttcctattatatcttcccaaattaccaatacattacactagcatctgaatttcataaccaatctcgatacaccaaatcgagatctgcagggatccccgatcatgcaaaaactcattaactcagtgcaaaactatgcctggggcagcaaaacggcgttgactgaactttatggtatggaaaatccgtccagccagccgatggccgagctgtggatgggcgcacatccgaaaagcagttcacgagtgcagaatgccgccggagatatcgtttcactgcgtgatgtgattgagagtgataaatcgactctgctcggagaggccgttgccaaacgctttggcgaactgcctttcctgttcaaagtattatgcgcagcacagccactctccattcaggttcatccaaacaaacacaattctgaaatcggttttgccaaagaaaatgccgcaggtatcccgatggatgccgccgagcgtaactataaagatcctaaccacaagccggagctggtttttgcgctgacgcctttccttgcgatgaacgcgtttcgtgaattttccgagattgtctccctactccagccggtcgcaggtgcacatccggcgattgctcactttttacaacagcctgatgccgaacgtttaagcgaactgttcgccagcctgttgaatatgcagggtgaagaaaaatcccgcgcgctggcgattttaaaatcggccctcgatagccagcagggtgaaccgtggcaaacgattcgtttaatttctgaattttacccggaagacagcggtctgttctccccgctattgctgaatgtggtgaaattgaaccctggcgaagcgatgttcctgttcgctgaaacaccgcacgcttacctgcaaggcgtggcgctggaagtgatggcaaactccgataacgtgctgcgtgcgggtctgacgcctaaatacattgatattccggaactggttgccaatgtgaaattcgaagccaaaccggctaaccagttgttgacccagccggtgaaacaaggtgcagaactggacttcccgattccagtggatgattttgccttctcgctgcatgaccttagtgataaagaaaccaccattagccagcagagtgccgccattttgttctgcgtcgaaggcgatgcaacgttgtggaaaggttctcagcagttacagcttaaaccgggtgaatcagcgtttattgccgccaacgaatcaccggtgactgtcaaaggccacggccgtttagcgcgtgtttacaacaagctgtaagagcttactgaaaaaattaacatctcttgctaagctgggagctcgtcgacggatcgaattcctgcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattgggtaccgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgggcggccagcatggccgtatccgcaatgtgttattaagttgtctaagcgtcaatttgtttacaccacaatatatcctgccaccagccagccaacagctccccgaccggcagctcggcacaaaatcaccactcgatacaggcagcccatcagaattaattctcatgtttgacagcttatcatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatccggctcgtataatgtgtggaattgtgagcggataacaatttcacacaggaaacagaccatgagggaagcgttgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctaggcaggcttatcttggacaagaagatcgcttggcctcgcgcgcagatcagttggaagaatttgttcactacgtgaaaggcgagatcaccaaagtagtcggcaaataaagctctagtggatctccgtacccccgggggatctggctcgcggcggacgcacgacgccggggcgagaccataggcgatctcctaaatcaatagtagctgtaacctcgaagcgtttcacttgtaacaacgattgagaatttttgtcataaaattgaaatacttggttcgcatttttgtcatccgcggtcagccgcaattctgacgaactgcccatttagctggagatgattgtacatccttcacgtgaaaatttctcaagcgctgtgaacaagggttcagattttagattgaaaggtgagccgttgaaacacgttcttcttgtcgatgacgacgtcgctatgcggcatcttattattgaataccttacgatccacgccttcaaagtgaccgcggtagccgacagcacccagttcacaagagtactctcttccgcgacggtcgatgtcgtggttgttgatctaaatttaggtcgtgaagatgggctcgagatcgttcgtaatctggcggcaaagtctgatattccaatcataattatcagtggcgaccgccttgaggagacggataaagttgttgcactcgagctaggagcaagtgattttatcgctaagccgttcagtatcagagagtttctagcacgcattcgggttgccttgcgcgtgcgccccaacgttgtccgctccaaagaccgacggtctttttgttttactgactggacacttaatctcaggcaacgtcgcttgatgtccgaagctggcggtgaggtgaaacttacggcaggtgagttcaatcttctcctcgcgtttttagagaaaccccgcgacgttctatcgcgcgagcaacttctcattgccagtcgagtacgcgacgaggaggtttatgacaggagtatagatgttctcattttgaggctgcgccgcaaacttgaggcagatccgtcaagccctcaactgataaaaacagcaagaggtgccggttatttctttgacgcggacgtgcaggtttcgcacggggggacgatggcagcctgagccaattcccagatccccgaggaatcggcgtgagcggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagctctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttgatccggaattaattcctgtggttggcatgcacatacaaatggacgaacggataaaccttttcacgcccttttaaatatccgattattctaataaacgctcttttctcttag

SEQ ID NO: 3 - DNA, 859 bp, Arabidopsis
  misc_feature (1)..(859): FLC cDNA fragment
atgggaagaaagaaattagaaatcaagcgaattgagaacaaaagtagccgacaagtcaccttctccaaacgtcgcaacggtctcatcgagaaagctcgtcagctttctgttctctgtgacgcatccgtcgctcttctcgtcgtctccgcctccggcaagctctacagcttctcctccggcgataacctggtcaagatccttgatcgatatgggaaacagcatgctgatgatcttaaagccttggatcatcagtcaaaagctctgaactatggttcacactatgagctacttgaacttgtggatagcaagcttgtgggatcaaatgtcaaaaatgtgagtatcgatgctcttgttcaactggaggaacaccttgagactgccctctccgtgactagagccaagaagaccgaactcatgttgaagcttgttgtgaatcttaaagaaaaggagaaaatgctgaaagaagagaaccaggttttggctagccagatggagaataatcatcatgtgggagcagaagctgagatggagatgtcacctgctggacaaatctccgacaatcttccggtgactctcccactacttaattaaccaccttaaatcggcggttgaaatcaaaatccaaaacatatataattatgaagaaaaaaaaataagatatgtaattattccgctgataagggcgagcgtatgtatatcttaatactctctctttggccaagagactttgtgtgtgatacttaagtagacggaactaagtcaatactatctgttttaagacaaaaggttgatgaactttgtaccttattcgtgtgagaattgcatcgagatcttgagtgtaaaaaaaaaaaaaaaaaaa

SEQ ID NO: 4 - DNA, 4818 bp, sugar beet
  misc_feature (1)..(4818): AGL20 partial gene
actaagacaattgatcggtaccaaaagcatatgaaggatctgcgtggagtgaaaagtaaagtgttggatcaaaaaaatttgctggtaaatttctattcattgtttatataaaaaggtagaatataattatattatatacttttcgattcataagcaagtctgaattatattcttcatatatttcactggaatttaaattcttttttttatgaataagcagaccaacatgtcaatccttttcaagataattaattaggtaattaggtgtcgatgtcatctaacttagtttgttaaacctcgaggtagtttttaagacttcaatggaaaaaagagaccgtaacttagtttgaaacaagatatgtcatctaacttggttgctaacattggatagtttttatgacttaaaaaggaaaaaagagaatgtaagtagtagcgtgcaaattgaaggtacagtaagcataagcattgttatgcatataaactaatcgtatataaatctaattggtaccagatttggtcttaatattgtattaatgaagtgcaagaaaagcaacaatctcatgttgtttatgttgaatattgttgttgtctcatgttgctcctgctagcttaggaagactcagatgcaccttttctttattttcttactttgtaaataacacttgcaattttaagcatggtaaattgggactagggttttaaagtattgatcatgagtcgggttggaccatatatcatcggtcaagttaggttatgtaagcgacataccaataaaatagcttgaccatttcaaaataggttgtaagtttcatcaatcgggtccatatcttctgtgatattacttactatctcaatataattactattcactataattttacaaatcatcgaaaaaataaatgttattatgtaaaataaaattggattcgtgctagtttatattgaaattatcaactttgcgtagttattgtaaatagatcgtaaaaatattaatagtcaaaatgaaacaacgacgaacatgatatgagggagtgctagatactagatgcaggggcggattcaggattctggaccaggagtagcgcaactttacattactagaaactcaaaaaaaaaagttcgtaattactaaattcaaatgttacaaaatcctaaatgttacaaatcaatcatatatcactaccaaatttccacaaattcaacccaattaacaatcatacttcacaacaatcaatcaaatttccacaaattcaacccaattaacaatcatacattcatacttcacaacaatcaatcaaatttctacaaattcaacccaattaacaatcatacattcatacttcacaacaatcaatcaaatttccacaaattcagcccaactaacaatgaaaaaaacgacaacaaccaatcaaaatttaacctcaatcaaaatttcaacccaatacccaataacaaataacaatcatacttcagtatttcacaacaatgaacaatcaaattccacagaccacaaattcagcctaactcttaacaaacaataaaaaatctgccaactagtaattccacataacacatacacatttaactcttaactaacaatgaaaaatctgccaaataacaatcttagaggaaagcaaaagaaagttacctgaacggaagtacggaagacggaatgtctgtagccctgaacgccggaactcgaaaggccgaaccgccgctgtgtcgccacttgctgtctcgccgaccaatttacacgaaaactgcgtctgccattagagtctagagagccgagaagaaagaaaaataaggttaagagaagactcaaatttagggctagatctagattacaattcgcggaaaagtaaacttaccattttctcggacctcggtttctccgacgcgaagacgatcgaaactgaagagttgagaaaaaacactccggcgagactcgattttccgacgacttgagagtttgagacttgagagagagagagagagagaggggacttgagacttgagagagcgaaattcccaaatgaaacacgctgctgaatttttttgcgatttttggcgattttttgaatttttttttttttttaaaagcaggggtagcacgtgctaccccttgttaccacgtggctccgccatgagtatctcaagcagttaaagaaagtattatactacaggtgagaacttttggaagagtggaaaggccaatcaactgcaagtgaatctgtatggtgtatccaagaccttgatatatatattcagaactccacaacaaaatatacttcctccgttttgtttttaatgcaataaagaggtattatttgtgagatataaaatattcatttgttgcatttaaaacgaaaccgaggaagtatgaatttataagaatttcccggaatcattgagtggagtttctatatgtccctgaatatctcaatctgtgtaactgcatgcattagcactttagcagcatttgtcaacttacagaatccgtatttttcatgcagcaattcaaggatgatactattgagcttgagaagaagttaaaaagtctcgaactttctaaacggttaggcaattcaatcaactaattgaatcatgcatgttctagctagctagctaactcttctacaacttatgcatgtttttatgggtttctaggaaattacttggagatggtctagaggcatgttccattgatgagcttcaagaattagaaaaacaattagataaaagtctttccagtatccgagcaagaaaggtacataatcttatctgaatttattggcgcttatctaaattaaaataaacttgtttagaacttacctgttgttatttatgcgaacttatttgtcttaaagcttattttttaaggtgaacagaataaatgctacatgcatatctgatctggtttaaccaatttactactgttttctcccagaatgcattgtacaaagagcagattaataagttgtatgaagaggtaagataaattcacaagttttactgcacaagttgattgtttttaggttattgaacgagctcagattaatgacatacgtcatttaccttcttttatagttgcatgttttcaaagacttcatttgttgtagtacttattttaaatattagacgtttgcttattttttccgttatatattttggatccgtaggagaagcacttaatcaaagaaaatgtccggttgaaagataaggtaagtagtttcatatcatacgttgcctaattgttttttttcttataggctccgtttcgtagggcgtaaaacgttttcccggaaaactgttttcctctattttcagttttacattgtttggttagcaaaagagtgtaaaaccattttcccttgggttaaatttactctcccaatgatggaaaaccattttcctttcaaaatgaagggaaaactattttccttatctctcttgtacactcttctcactacctccttactttccctttcattttcctttgacttcatcatttttattacatcgaaccaaacaacggaaaactaattttggaattgtgttttccattgtaaattattttccatgaaaatcattttacactgaaaatgttttacgccctaccaaacggagtcatagtgtaagtattcctggattgttgcatgtcctacatttggcgagtaacggacaaccgttatgatcattttattcgcaatattgatttgatgcggaagtatgcttttgatagaagacaaaacatgtgattttgtgttgaattggagtacatggcaactactttagttggtaaaatcttatgagatgaagtccataaggcttgaaattgaatctcgttcatatttttgtggttatatacgcactcaaaagaagtgaatctatatattaaactaaatgatggtagatgagaatgccttttaataataataggttatatgtaattagaggtcattctatataattgatttataataaatatgagtatctttcgatatttctgcttctatttccccatgccacaagtagtagtctttatcagaaaatttcgtaaaaattatatgaggaagcatataccaagtagtacttgatatagtgaaagaaataatctaacaatcttaagattgtgagataaagccctagtaatggtactaggaattccccttactttaccccctacttttattttaaaggggccttgcctgataagctgaattgggtcgacctatgaaaggttttggtttcctagggattagggcacttctttttaccatatatataaataaaaaaaaagagggagcgtaagtggactagtacattaggatgatcaagtttagtataaatcatacctaaagatagtatagaaattaaggttttgttcacttatgttaaacacacgttttttcatgagctattgtagtattgtatcaatgtatctcatgatgttgccctataattacaacatgtacttgattctctttccaaaagtatacaataagatgtggttcatctcataatataggggaatataatgatgatgctcttttattcgatcaactttgatatgtatttgttagtttcgatttctataatgtatgattttgtattataatttaattttatttaaaatgttgcgcatttaacttagaaaaaaacttgtgttgtaaataaagtatccatcataacacatatctggatacatactctgtgcatgaatgttttataccctacaaataatagcgaaattctaatgggtgattgctaaatccaacttctcaggttctcaattcttcaccagatctgctacct

SEQ ID NO: 5 - DNA, 283 bp, Sugar beet
  misc_feature (1)..(283): BvAGL20 repeat fragment, first and/or second strand
aagaagttaaaaagtctcgaactttctaaacggaaattacttggagatggtctagaggcatgttccattgatgagcttcaagaattagaaaaacaattagataaaagtctttccagtatccgagcaagaaagaatgcattgtacaaagagcagattaataagttgtatgaagaggagaagcacttaatcaaagaaaatgtccggttgaaagataaggttctcaattcttcaccagatctgctacgtcgccaacaacgagcagatgatcaggagatcagcatgc

SEQ ID NO: 6 - DNA, 611 bp, Sugar beet
  misc_feature (1)..(611): BvAGL20 cDNA fragment
atggtkmgrggnaaracncagatgaagagaattgaaaatgattcaagcagacaagtgacttcctcaaaaagaagaaatgggttgttgaagaaagcttttgagctttcagttctttgtgatgctgaagttgcacttatcattttttctccttctggaaaactctatgaattcgcaagctcaagtacgactaagacaattgatcggtaccaaaagcatatgaaggatctgcgtggagtgaaaagtaaagtgttggatcaaaaaaatttgctgcaattcaaggatgatactattgagcttgagaagaagttaaaaagtctcgaactttctaaacggaaattacttggagatggtctagaggcatgttccattgatgagcttcaagaattagaaaaacaattcgataaaggtctttccagtctccgagcaagaaagaatgcattgtacaaagagcagattaataagttgtatgaagaggacaagcacttaatcaaagaaaatgtccggttgaaagataaggttctcaattcttcaccagatctgctacctcgccaacaacgagcagatgatcaggagatcagcatgcaagawgtngagacnsaaytgttcatygg

SEQ ID NO: 7 - DNA, 22 bp, Artificial
  HiNK529 primer
ccgcggacctgcacatcaacaa

SEQ ID NO: 8 - DNA, 25 bp, Artificial
  HiNK792 primer
gatcaggagatcagcatgcggatcc

SEQ ID NO: 9 - DNA, 39 bp, Artificial
  HiNK793 primer
gaagcagaaacttacctaagaagttaaaaagtctcgaac

SEQ ID NO: 10 - DNA, 28 bp, Artificial
  HiNK794 primer
gttcgagactttttaacttcttccgcgg

SEQ ID NO: 11 - DNA, 25 bp, Artificial
  HiNK795 primer
gtcgacgcatgctgatctcctgatc

SEQ ID NO: 12 - DNA, 42 bp, Artificial
  HiNK796 primer
gtagaagcagaaacttacctaagaagttaaaaagtctcgaac

SEQ ID NO: 13 - DNA, 25 bp, Artificial
  HiNK624 primer
atggtkmgrggnaaracncagatga

SEQ ID NO: 14 - DNA, 26 bp, Artificial
  HiNK619 primer
ccratgaacarttsngtctcnacwtc

SEQ ID NO: 15 - DNA, 28 bp, Artificial
  HiNK725 primer
actaagacaattgatcggtaccaaaagc

SEQ ID NO: 16 - DNA, 28 bp, Artificial
  HiNK729 primer
aaggtagcagatctggtgaagaattgag

SEQ ID NO: 17 - DNA, 33 bp, Artificial
  HiNK2617 Primer
taaatggatccaagaagttaaaaagtctcgaac

SEQ ID NO: 18 - DNA, 45 bp, Artificial
  HiNK2618 primer
gaagcagaaacttacctgtcgacaagaagttaaaaagtctcgaac

SEQ ID NO: 19 - DNA, 10569 bp, Artificial
  pHiNK440
gtttacccgccaatatatcctgtcaaacactgatagtttaaactgaaggcgggaaacgacaatctgatcatgagcggagaattaagggagtcacgttatgacccccgccgatgacgcgggacaagccgttttacgtttggaactgacagaaccgcaacgctgcaggaattggccgcagcggccatttaaatcaattgggcgcgccgaattcgagctcggtacccggggatcctctagatcatgtttgacagcttatcatcggatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattagatgataatcatcgcaagaccggcaacaggattcaatcttaagaaactttattgccaaatgtttgaacgatctctgcaggtcgacgcatgctgatctcctgatcatctgctcgttgttggcgacgtagcagatctggtgaagaattgagaaccttatctttcaaccggacattttctttgattaagtgcttctcctcttcatacaacttattaatctgctctttgtacaatgcattctttcttgctcggatactggaaagacttttatctaattgtttttctaattcttgaagctcatcaatggaacatgcctctagaccatctccaagtaatttccgtttagaaagttcgagactttttaacttcttggatccaaagagagagtcgcgagagatttgcagagatcgctttaggctttgggagagattgaagagtcagaaaaagacgaaaggatgaattattatcttccacacgaaggtcttctttatatcgcaaaccaaaagcccaaaaccgtcttttctattaatgagaataaaatatctttagccaaaacaaaaaaaggaagatatcagttgaggattattatcacgaaactaaaggaaggaatcatatgatacgtgtcatattttccaccgtgcgtttttaaaagaccgactcaagtagagacatcctatggtggtggttggattaggtcatccattacatctgcttcactgacatttttctatttttctttttgtatatacttttcctcaaataatttctttcttttctatagaagaatttaatcaataaggaaaaagttcaaaaaagattctttccattaagactatgtcttggttaacccaacccattaagaataagcaatcataatatatatagagaatactaatactatatatgagatttttcttttaatttcatgttgattatgatagtttatcttcttgatttaatttatcaatacttggcataaaagattctaatctactctaataaagaaaagaaaaaaaagtatctaccattgactaattaaaataaggaaacttatctaccaaatttgagtattttttagaacaatctttttggtttaattccaaaactctaaacctaattgttgggaaaaaggacctaatttttaagaaaagttaataattagaagatctgtatgttttttttttgatccaagtttttatttcttttctctttttttcatgataaaatctatgtttttttagtctacaattaaagtaattgttattattttctttatctttttttgttgttgttgttaattccctttttttttttttaacagcaacttcttaaaaaaaaaaacagttgggccttgaatttatttcaggcctgcgttattaagcccagataataactcaaaacaaaaaaaatgttgaaccggaataaacccgcgagattaaatgccggttttcaggtaacatagaagaagaatatatgaggattgaagaagtattcaagaggcggaacaattcacaagtccaagagcttaaatttctcctcactcttctgctacagactcggaactctttctctttgctaaaataagatgttcaggatttttgttgcccgacaattcatgtatctcacactctctctcttctctgttcttactactctgttacattaccaccaactcaagactttcttccacaatggcgtttatgagacttggctccaaatccggacggatctctagagtcgaccatggtgatcactgcaggcatgcaagcttcgtacgttaattaattcgaatccggagcggccgcacgcgtgggcccgtttaaacctcgagagatctgctagcatcgatggtaccgagctcgagactagctacaggccaaattcgctcttagccgtacaatattactcaccggtgcgatgccccccatcgtaggtgaaggtggaaattaatgatccatcttgagaccacaggcccacaacagctaccagtttcctcaagggtccaccaaaaacgtaagcgcttacgtacatggtcgataagaaaaggcaatttgtagatgttaacatccaacgtcgctttcagggatcccgaattccaagcttggaattcgggatcctacaggccaaattcgctcttagccgtacaatattactcaccggtgcgatgccccccatcgtaggtgaaggtggaaattaatgatccatcttgagaccacaggcccacaacagctaccagtttcctcaagggtccaccaaaaacgtaagcgcttacgtacatggtcgataagaaaaggcaatttgtagatgttaacatccaacgtcgctttcagggatcccgaattccaagcttggaattcgggatcctacaggccaaattcgctcttagccgtacaatattactcaccggtgcgatccccccatcgtaggtgaaggtggaaattaatgatccatcttgagaccacaggcccacaacagctaccagtttcctcaagggtccaccaaaaacgtaagcgcttacgtacatggtcgataagaaaaggcaatttgtagatgttaacatccaacgtcgctttcagggatcccgaattccaagcttgggctgcaggtcaatcccattgcttttgaagcagctcaacattgatctctttctcgagggagatttttcaaatcagtgcgcaagacgtgacgtaagtatccgagtcagtttttatttttctactaatttggtcgtttatttcggcgtgtaggacatggcaaccgggcctgaatttcgcgggtattctgtttctattccaactttttcttgatccgcagccattaacgacttttgaatagatacgctgacacgccaagcctcgctagtcaaaagtgtaccaaacaacgctttacagcaagaacggaatgcgcgtgacgctcgcggtgacgccatttcgccttttcagaaatggataaatagccttgcttcctattatatcttcccaaattaccaatacattacactagcatctgaatttcataaccaatctcgatacaccaaatcgagatctgcagggatccccgatcatgcaaaaactcattaactcagtgcaaaactatgcctggggcagcaaaacggcgttgactgaactttatggtatggaaaatccgtccagccagccgatggccgagctgtggatgggcgcacatccgaaaagcagttcacgagtgcagaatgccgccggagatatcgtttcactgcgtgatgtgattgagagtgataaatcgactctgctcggagaggccgttgccaaacgctttggcgaactgcctttcctgttcaaagtattatgcgcagcacagccactctccattcaggttcatccaaacaaacacaattctgaaatcggttttgccaaagaaaatgccgcaggtatcccgatggatgccgccgagcgtaactataaagatcctaaccacaagccggagctggtttttgcgctgacgcctttccttgcgatgaacgcgtttcgtgaattttccgagattgtctccctactccagccggtcgcaggtgcacatccggcgattgctcactttttacaacagcctgatgccgaacgtttaagcgaactgttcgccagcctgttgaatatgcagggtgaagaaaaatcccgcgcgctggcgattttaaaatcggccctcgatagccagcagggtgaaccgtggcaaacgattcgtttaatttctgaattttacccggaagacagcggtctgttctccccgctattgctgaatgtggtgaaattgaaccctggcgaagcgatgttcctgttcgctgaaacaccgcacgcttacctgcaaggcgtggcgctggaagtgatggcaaactccgataacgtgctgcgtgcgggtctgacgcctaaatacattgatattccggaactggttgccaatgtgaaattcgaagccaaaccggctaaccagttgttgacccagccggtgaaacaaggtgcagaactggacttcccgattccagtggatgattttgccttctcgctgcatgaccttagtgataaagaaaccaccattagccagcagagtgccgccattttgttctgcgtcgaaggcgatgcaacgttgtggaaaggttctcagcagttacagcttaaaccgggtgaatcagcgtttattgccgccaacgaatcaccggtgactgtcaaaggccacggccgtttagcgcgtgtttacaacaagctgtaagagcttactgaaaaaattaacatctcttgctaagctgggagctcgtcgacggatcgaattcctgcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattgggtaccgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgggcggccagcatggccgtatccgcaatgtgttattaagttgtctaagcgtcaatttgtttacaccacaatatatcctgccaccagccagccaacagctccccgaccggcagctcggcacaaaatcaccactcgatacaggcagcccatcagaattaattctcatgtttgacagcttatcatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatccggctcgtataatgtgtggaattgtgagcggataacaatttcacacaggaaacagaccatgagggaagcgttgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctaggcaggcttatcttggacaagaagatcgcttggcctcgcgcgcagatcagttggaagaatttgttcactacgtgaaaggcgagatcaccaaagtagtcggcaaataaagctctagtggatctccgtacccccgggggatctggctcgcggcggacgcacgacgccggggcgagaccataggcgatctcctaaatcaatagtagctgtaacctcgaagcgtttcacttgtaacaacgattgagaatttttgtcataaaattgaaatacttggttcgcatttttgtcatccgcggtcagccgcaattctgacgaactgcccatttagctggagatgattgtacatccttcacgtgaaaatttctcaagcgctgtgaacaagggttcagattttagattgaaaggtgagccgttgaaacacgttcttcttgtcgatgacgacgtcgctatgcggcatcttattattgaataccttacgatccacgccttcaaagtgaccgcggtagccgacagcacccagttcacaagagtactctcttccgcgacggtcgatgtcgtggttgttgatctaaatttaggtcgtgaagatgggctcgagatcgttcgtaatctggcggcaaagtctgatattccaatcataattatcagtggcgaccgccttgaggagacggataaagttgttgcactcgagctaggagcaagtgattttatcgctaagccgttcagtatcagagagtttctagcacgcattcgggttgccttgcgcgtgcgccccaacgttgtccgctccaaagaccgacggtctttttgttttactgactggacacttaatctcaggcaacgtcgcttgatgtccgaagctggcggtgaggtgaaacttacggcaggtgagttcaatcttctcctcgcgtttttagagaaaccccgcgacgttctatcgcgcgagcaacttctcattgccagtcgagtacgcgacgaggaggtttatgacaggagtatagatgttctcattttgaggctgcgccgcaaacttgaggcagatccgtcaagccctcaactgataaaaacagcaagaggtgccggttatttctttgacgcggacgtgcaggtttcgcacggggggacgatggcagcctgagccaattcccagatccccgaggaatcggcgtgagcggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagctctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttgatccggaattaattcctgtggttggcatgcacatacaaatggacgaacggataaaccttttcacgcccttttaaatatccgattattctaataaacgctcttttctcttag

SEQ ID NO: 20 - DNA, 10569 bp, Artificial
  pHiNK441
gtttacccgccaatatatcctgtcaaacactgatagtttaaactgaaggcgggaaacgacaatctgatcatgagcggagaattaagggagtcacgttatgacccccgccgatgacgcgggacaagccgttttacgtttggaactgacagaaccgcaacgctgcaggaattggccgcagcggccatttaaatcaattgggcgcgccgaattcgagctcggtacccggggatcctctagatcatgtttgacagcttatcatcggatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattagatgataatcatcgcaagaccggcaacaggattcaatcttaagaaactttattgccaaatgtttgaacgatctctgcaggtcgacaagaagttaaaaagtctcgaactttctaaacggaaattacttggagatggtctagaggcatgttccattgatgagcttcaagaattagaaaaacaattagataaaagtctttccagtatccgagcaagaaagaatgcattgtacaaagagcagattaataagttgtatgaagaggagaagcacttaatcaaagaaaatgtccggttgaaagataaggttctcaattcttcaccagatctgctacgtcgccaacaacgagcagatgatcaggagatcagcatgcggatccaaagagagagtcgcgagagatttgcagagatcgctttaggctttgggagagattgaagagtcagaaaaagacgaaaggatgaattattatcttccacacgaaggtcttctttatatcgcaaaccaaaagcccaaaaccgtcttttctattaatgagaataaaatatctttagccaaaacaaaaaaaggaagatatcagttgaggattattatcacgaaactaaaggaaggaatcatatgatacgtgtcatattttccaccgtgcgtttttaaaagaccgactcaagtagagacatcctatggtggtggttggattaggtcatccattacatctgcttcactgacatttttctatttttctttttgtatatacttttcctcaaataatttctttcttttctatagaagaatttaatcaataaggaaaaagttcaaaaaagattctttccattaagactatgtcttggttaacccaacccattaagaataagcaatcataatatatatagagaatactaatactatatatgagatttttcttttaatttcatgttgattatgatagtttatcttcttgatttaatttatcaatacttggcataaaagattctaatctactctaataaagaaaagaaaaaaaagtatctaccattgactaattaaaataaggaaacttatctaccaaatttgagtattttttagaacaatctttttggtttaattccaaaactctaaacctaattgttgggaaaaaggacctaatttttaagaaaagttaataattagaagatctgtatgttttttttttgatccaagtttttatttcttttctctttttttcatgataaaatctatgtttttttagtctacaattaaagtaattgttattattttctttatctttttttgttgttgttgttaattccctttttttttttttaacagcaacttcttaaaaaaaaaaacagttgggccttgaatttatttcaggcctgcgttattaagcccagataataactcaaaacaaaaaaaatgttgaaccggaataaacccgcgagattaaatgccggttttcaggtaacatagaagaagaatatatgaggattgaagaagtattcaagaggcggaacaattcacaagtccaagagcttaaatttctcctcactcttctgctacagactcggaactctttctctttgctaaaataagatgttcaggatttttgttgcccgacaattcatgtatctcacactctctctcttctctgttcttactactctgttacattaccaccaactcaagactttcttccacaatggcgtttatgagacttggctccaaatccggacggatctctagagtcgaccatggtgatcactgcaggcatgcaagcttcgtacgttaattaattcgaatccggagcggccgcacgcgtgggcccgtttaaacctcgagagatctgctagcatcgatggtaccgagctcgagactagctacaggccaaattcgctcttagccgtacaatattactcaccggtgcgatgccccccatcgtaggtgaaggtggaaattaatgatccatcttgagaccacaggcccacaacagctaccagtttcctcaagggtccaccaaaaacgtaagcgcttacgtacatggtcgataagaaaaggcaatttgtagatgttaacatccaacgtcgctttcagggatcccgaattccaagcttggaattcgggatcctacaggccaaattcgctcttagccgtacaatattactcaccggtgcgatgccccccatcgtaggtgaaggtggaaattaatgatccatcttgagaccacaggcccacaacagctaccagtttcctcaagggtccaccaaaaacgtaagcgcttacgtacatggtcgataagaaaaggcaatttgtagatgttaacatccaacgtcgctttcagggatcccgaattccaagcttggaattcgggatcctacaggccaaattcgctcttagccgtacaatattactcaccggtgcgatccccccatcgtaggtgaaggtggaaattaatgatccatcttgagaccacaggcccacaacagctaccagtttcctcaagggtccaccaaaaacgtaagcgcttacgtacatggtcgataagaaaaggcaatttgtagatgttaacatccaacgtcgctttcagggatcccgaattccaagcttgggctgcaggtcaatcccattgcttttgaagcagctcaacattgatctctttctcgagggagatttttcaaatcagtgcgcaagacgtgacgtaagtatccgagtcagtttttatttttctactaatttggtcgtttatttcggcgtgtaggacatggcaaccgggcctgaatttcgcgggtattctgtttctattccaactttttcttgatccgcagccattaacgacttttgaatagatacgctgacacgccaagcctcgctagtcaaaagtgtaccaaacaacgctttacagcaagaacggaatgcgcgtgacgctcgcggtgacgccatttcgccttttcagaaatggataaatagccttgcttcctattatatcttcccaaattaccaatacattacactagcatctgaatttcataaccaatctcgatacaccaaatcgagatctgcagggatccccgatcatgcaaaaactcattaactcagtgcaaaactatgcctggggcagcaaaacggcgttgactgaactttatggtatggaaaatccgtccagccagccgatggccgagctgtggatgggcgcacatccgaaaagcagttcacgagtgcagaatgccgccggagatatcgtttcactgcgtgatgtgattgagagtgataaatcgactctgctcggagaggccgttgccaaacgctttggcgaactgcctttcctgttcaaagtattatgcgcagcacagccactctccattcaggttcatccaaacaaacacaattctgaaatcggttttgccaaagaaaatgccgcaggtatcccgatggatgccgccgagcgtaactataaagatcctaaccacaagccggagctggtttttgcgctgacgcctttccttgcgatgaacgcgtttcgtgaattttccgagattgtctccctactccagccggtcgcaggtgcacatccggcgattgctcactttttacaacagcctgatgccgaacgtttaagcgaactgttcgccagcctgttgaatatgcagggtgaagaaaaatcccgcgcgctggcgattttaaaatcggccctcgatagccagcagggtgaaccgtggcaaacgattcgtttaatttctgaattttacccggaagacagcggtctgttctccccgctattgctgaatgtggtgaaattgaaccctggcgaagcgatgttcctgttcgctgaaacaccgcacgcttacctgcaaggcgtggcgctggaagtgatggcaaactccgataacgtgctgcgtgcgggtctgacgcctaaatacattgatattccggaactggttgccaatgtgaaattcgaagccaaaccggctaaccagttgttgacccagccggtgaaacaaggtgcagaactggacttcccgattccagtggatgattttgccttctcgctgcatgaccttagtgataaagaaaccaccattagccagcagagtgccgccattttgttctgcgtcgaaggcgatgcaacgttgtggaaaggttctcagcagttacagcttaaaccgggtgaatcagcgtttattgccgccaacgaatcaccggtgactgtcaaaggccacggccgtttagcgcgtgtttacaacaagctgtaagagcttactgaaaaaattaacatctcttgctaagctgggagctcgtcgacggatcgaattcctgcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattgggtaccgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgggcggccagcatggccgtatccgcaatgtgttattaagttgtctaagcgtcaatttgtttacaccacaatatatcctgccaccagccagccaacagctccccgaccggcagctcggcacaaaatcaccactcgatacaggcagcccatcagaattaattctcatgtttgacagcttatcatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatccggctcgtataatgtgtggaattgtgagcggataacaatttcacacaggaaacagaccatgagggaagcgttgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctaggcaggcttatcttggacaagaagatcgcttggcctcgcgcgcagatcagttggaagaatttgttcactacgtgaaaggcgagatcaccaaagtagtcggcaaataaagctctagtggatctccgtacccccgggggatctggctcgcggcggacgcacgacgccggggcgagaccataggcgatctcctaaatcaatagtagctgtaacctcgaagcgtttcacttgtaacaacgattgagaatttttgtcataaaattgaaatacttggttcgcatttttgtcatccgcggtcagccgcaattctgacgaactgcccatttagctggagatgattgtacatccttcacgtgaaaatttctcaagcgctgtgaacaagggttcagattttagattgaaaggtgagccgttgaaacacgttcttcttgtcgatgacgacgtcgctatgcggcatcttattattgaataccttacgatccacgccttcaaagtgaccgcggtagccgacagcacccagttcacaagagtactctcttccgcgacggtcgatgtcgtggttgttgatctaaatttaggtcgtgaagatgggctcgagatcgttcgtaatctggcggcaaagtctgatattccaatcataattatcagtggcgaccgccttgaggagacggataaagttgttgcactcgagctaggagcaagtgattttatcgctaagccgttcagtatcagagagtttctagcacgcattcgggttgccttgcgcgtgcgccccaacgttgtccgctccaaagaccgacggtctttttgttttactgactggacacttaatctcaggcaacgtcgcttgatgtccgaagctggcggtgaggtgaaacttacggcaggtgagttcaatcttctcctcgcgtttttagagaaaccccgcgacgttctatcgcgcgagcaacttctcattgccagtcgagtacgcgacgaggaggtttatgacaggagtatagatgttctcattttgaggctgcgccgcaaacttgaggcagatccgtcaagccctcaactgataaaaacagcaagaggtgccggttatttctttgacgcggacgtgcaggtttcgcacggggggacgatggcagcctgagccaattcccagatccccgaggaatcggcgtgagcggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagctctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttgatccggaattaattcctgtggttggcatgcacatacaaatggacgaacggataaaccttttcacgcccttttaaatatccgattattctaataaacgctcttttctcttag

SEQ ID NO: 21 - DNA, 702 bp, Beta vulgaris
  CDS (63)..(662)
aaaggacagagagagtgagagaaattgcagcgacgaagaaagagaaaggtatttggataaggatgggaagaaggaagatagagatgaaaagaattgaagataaaagtagtcgtcaagttacattttcaaagcggcgttctggtcttatcaaaaaagctcgcgaactctctatcctttgtgatgtcgatgttgctgttcttgttttctctaatcgtggtcgtctttacgaattcgtcaatagttcttcttcttccagtttgtctcagattcttaagcgctatcaagattccactgcagcagacgggaaagcttcaatagctgctgttgaaacagagagttcaccttctagttgtgcagaagtccaaacatgtggtgagctagtaaaatcagttgaaaggtacctagaaggaccagagcttgaaaatcttaggcttgaggacttcatgaggctggagaggcaactagctgatgcccttgtacagaccagaacccgaaaggagaagctgttgaaacaagagaatgaacagttgaaggatgaggtagcaaatctgataggcattcccaagagccgaaaccataaggatttaggggttaacaacttgatggaggtggatgctgatagacaatactctcagccactcagaacacttccactgctgaggtaactgctgtaagagtcggcattgagcagcattttgsmct

SEQ ID NO: 22 - protein, 200 aa, Beta vulgaris
MGRRKIEMKRIEDKSSRQVTFSKRRSGLIKKARELSILCDVDVAVLVFSNRGRLYEFVNSSSSSSLSQILKRYQDSTAADGKASIAAVETESSPSSCAEVQTCGELVKSVERYLEGPELENLRLEDFMRLERQLADALVQTRTRKEKLLKQENEQLKDEVANLIGIPKSRNHKDLGVNNLMEVDADRQYSQPLRTLPLLR

SEQ ID NO: 23 - DNA, 747 bp, Beta vulgaris
  CDS (63)..(707)
aaaggacagagagagtgagagaaattgcagcgacgaagaaagagaaaggtatttggataaggatgggaagaaggaagatagagatgaaaagaattgaagataaaagtagtcgtcaagttacattttcaaagcggcgttctggtcttatcaaaaaagctcgcgaactctctatcctttgtgatgtcgatgttgctgttcttgttttctctaatcgtggtcgtctttacgaattcgtcaatagttcttcttcttccagtttgtctcagattcttaagcgctatcaagattccactgcagcagacgggaaagcttcaatagctgctgttgaaacagagcagagttcaccttctagttgtgcagaagtccaaacatgtggtgagctagtaaaatcagttgaaaggtacctagaaggaccagagcttgaaaatcttaggcttgaggacttcatgaggctggagaggcaactagctgatgcccttgtacagaccagaacccgaaagactcaacttatgctagaatctatcggaacactaagtgaacaggagaagctgttgaaacaagagaatgaacagttgaaggatgaggtagcaaatctgataggcattcccaagagccgaaaccataaggatttaggggttaacaacttgatggaggtggatgctgatagacaatactctcagccactcagaacacttccactgctgaggtaactgctgtaagagtcggcattgagcagcattttgsmct

SEQ ID NO: 24 - protein, 215 aa, Beta vulgaris
MGRRKIEMKRIEDKSSRQVTFSKRRSGLIKKARELSILCDVDVAVLVFSNRGRLYEFVNSSSSSSLSQILKRYQDSTAADGKASIAAVETEQSSPSSCAEVQTCGELVKSVERYLEGPELENLRLEDFMRLERQLADALVQTRTRKTQLMLESIGTLSEQEKLLKQENEQLKDEVANLIGIPKSRNHKDLGVNNLMEVDADRQYSQPLRTLPLLR

SEQ ID NO: 25 - DNA, 705 bp, Beta vulgaris
  CDS (63)..(665)
aaaggacagagagagtgagagaaattgcagcgacgaagaaagagaaaggtatttggataaggatgggaagaaggaagatagagatgaaaagaattgaagataaaagtagtcgtcaagttacattttcaaagcggcgttctggtcttatcaaaaaagctcgcgaactctctatcctttgtgatgtcgatgttgctgttcttgttttctctaatcgtggtcgtctttacgaattcgtcaatagttcttcttcttccagtttgtctcagattcttaagcgctatcaagattccactgcagcagacgggaaagcttcaatagctgctgttgaaacagagcagagttcaccttctagttgtgcagaagtccaaacatgtggtgagctagtaaaatcagttgaaaggtacctagaaggaccagagcttgaaaatcttaggcttgaggacttcatgaggctggagaggcaactagctgatgcccttgtacagaccagaacccgaaaggagaagctgttgaaacaagagaatgaacagttgaaggatgaggtagcaaatctgataggcattcccaagagccgaaaccataaggatttaggggttaacaacttgatggaggtggatgctgatagacaatactctcagccactcagaacacttccactgctgaggtaactgctgtaagagtcggcattgagcagcattttgsmct

SEQ ID NO: 26 - protein, 201 aa, Beta vulgaris
MGRRKIEMKRIEDKSSRQVTFSKRRSGLIKKARELSILCDVDVAVLVFSNRGRLYEFVNSSSSSSLSQILKRYQDSTAADGKASIAAVETEQSSPSSCAEVQTCGELVKSVERYLEGPELENLRLEDFMRLERQLADALVQTRTRKEKLLKQENEQLKDEVANLIGIPKSRNHKDLGVNNLMEVDADRQYSQPLRTLPLLR

SEQ ID NO: 27 - DNA, 702 bp, Beta vulgaris; identical to SEQ ID NO: 21

SEQ ID NO: 28 - DNA, 682 bp, Beta vulgaris
aaaggacagagagagtgagagaaattgcagcgacgaagaaagagaaaggtatttggataaggatgggaagaaggaagatagagatgaaaagaattgaagataaaagtagtcgtcaagttacattttcaaagcggcgttctggtcttatcaaaaaagctcgcgaactctctatcctttgtgatgtcgatgttgctgttcttgttttctctaatcgtggtcgtctttacgaattcgtcaatagttcttcttcttccagtttgtctcagattcttaagcgctatcaagattccactgcagcagacgggaaagcttcaatagctgctgttgaaacagagcagagttcaccttctagttgtgcagaagtccaaacatgtggtgagctagtaaaatcagttgaaaggtacctagaaggaccagagcttgaaaatcttaggcttgaggacttcatgaggctggagaggcaactagctgatgcccttgtacagaccagaacccgaaagactcaacttatgctagaatctatcggaacactaagtgaacaggagaagctgttgaaacaagagaatgaacagttgaaggatgaggtagcaaatctgataggcattcccaagagccgaaaccataaggatttaggggttaacaacttgatggaggtggatgctgatagacaatactctcagcc

SEQ ID NO: 29 - DNA, 640 bp, Beta vulgaris
aaaggacagagagagtgagagaaattgcagcgacgaagaaagagaaaggtatttggataaggatgggaagaaggaagatagagatgaaaagaattgaagataaaagtagtcgtcaagttacattttcaaagcggcgttctggtcttatcaaaaaagctcgcgaactctctatcctttgtgatgtcgatgttgctgttcttgttttctctaatcgtggtcgtctttacgaattcgtcaatagttcttcttcttccagtttgtctcagattcttaagcgctatcaagattccactgcagcagacgggaaagcttcaatagctgctgttgaaacagagcagagttcaccttctagttgtgcagaagtccaaacatgtggtgagctagtaaaatcagttgaaaggtacctagaaggaccagagcttgaaaatcttaggcttgaggacttcatgaggctggagaggcaactagctgatgcccttgtacagaccagaacccgaaaggagaagctgttgaaacaagagaatgaacagttgaaggatgaggtagcaaatctgataggcattcccaagagccgaaaccataaggatttaggggttaacaacttgatggaggtggatgctgatagacaatactctcagcc

SEQ ID NO: 30 - DNA, 26 bp, Artificial sequence
  HiNK5277
cgncgnaayggnctnctnaaraargc

SEQ ID NO: 31 - DNA, 32 bp, Artificial sequence
  HiNK5279
gcntaygarctntcngtnctntgygaygcnga

SEQ ID NO: 32 - DNA, 21 bp, Artificial sequence
  AGL20 A
gtctcgaactttctaaacgga

SEQ ID NO: 33 - DNA, 20 bp, Artificial sequence
  AGL20 B
gatcatctgctcgttgttgg

SEQ ID NO: 34 - DNA, 22 bp, Artificial sequence
  HiNK023
cgcaagaccggcaacaggattc

SEQ ID NO: 35 - DNA, 23 bp, Artificial sequence
  gapCex5/6F
gctgctgctcacttgaagggtgg

SEQ ID NO: 36 - DNA, 20 bp, Artificial sequence
  gapCex8R
cttccacctctccagtcctt

SEQ ID NO: 37 - DNA, 25 bp, Artificial sequence
  HiNK 819
tctgcgtggagtgaaaagtaaagtg

(19) United States
(12) Patent Application Publication
(10) Pub. No.: US 2009/0205073 A1
(43) Pub. Date: Aug. 13, 2009

(54) TRANSGENIC PLANTS AND METHODS FOR CONTROLLING BOLTING IN SUGAR BEET

(75) Inventors: Johannes Jacobus Ludgerus Gielen, Bouloc (FR); Petronella Maria Van Roggen, Landskrona (SE); Signe Irene Elisabet Wremert Weich, Landskrona (SE)

Correspondence Address: SYNGENTA BIOTECHNOLOGY, INC.;PATENT DEPARTMENT, 3054 CORNWALLIS ROAD, P.O. BOX 12257, RESEARCH TRIANGLE PARK, NC, 27709-2257, US

(73) Assignee: Syngenta Participations AG

(21) Appl. No.: 12/297,908

(22) PCT Filed: Apr. 4, 2007
(86) PCT No.: PCT/EP2007/053325

§ 371 Date: Jan. 30, 2009

(30) Foreign Application Priority Data
Apr. 21, 2006 (EP) 06290670.6
Jan. 9, 2007 (EP) 07290025.1

(51) Int. Cl. C12N 15/82; A01H 5/00; C12Q 1/68; C12P 7/10; C10L 1/18

(52) U.S. Cl. 800/278; 800/298; 435/6; 435/165; 44/307

(57) ABSTRACT

This invention relates to the field of sugar beet bolting and flowering control, specifically methods and transgenic sugar beet plants for suppressing the vernalization response. In particular, the present invention includes sugar beet plants and methods for modulating sugar beet vernalization by over expression of an FLC gene or by suppressing AGL20 gene expression.

### FIELD OF THE INVENTION

This invention relates to the field of sugar beet bolting and flowering control, specifically to methods and transgenic sugar beet plants for suppressing the vernalization response.

### BACKGROUND OF THE INVENTION

Sugar beet has been cultivated for thousands of years as a sweets source, but its potential as a source of sugar was not discovered until the 18^{th} century. The sugar beet is a biennial plant belonging to the Chenopodiaceae. Its usual life cycle is completed in two years. In the first year a large succulent root is developed, which serves as a reserve for energy in the form of sucrose. For this reason it is farmed as an annual. In the second year it produces flowers and seeds. If there happens to be prolonged cool periods in the first year, the seed stalk can already sprout. This genetically determined thermal induction leads to a phenomenon called bolting. Cropping the beet for sugar extraction cuts the biennial cycle in half, whilst the sucrose is at its peak.

As already mentioned an obligate part of the complete sugar beet life cycle is the cold-induced vernalization, which induces bolting of the plants. The likelihood of bolting is increased in relationship to the number of days on which the maximum temperature does not exceed 12° C. This can lead to loss of yield when the early sowing method is applied, as 1% bolters in a crop have been estimated to reduce sugar yield by 0.4-0.7%.

There exist two methods for cropping sugar beet, spring and autumn cropping, whereas they are practiced in the southern, milder, climate or in northern latitudes respectively. Both rely on varieties with different degrees of bolting resistance. Bolting resistance influences temperature, length and irradiation limits tolerable for seed stalk induction and is a key trait in sugar beet breeding. To allow for complete control of bolting and flowering, by either blocking vernalization, devernalizing vernalized plants or suppressing flower or viable seed production would allow the sugar beet crop to be sown in autumn in northern latitudes without the risk of bolting and flowering in the following season. This shift from a spring into a winter crop would permit growers to drill their crop in autumn and to harvest the next summer. Comparison of winter to spring cultivars in crops like wheat and oilseed rape has shown that winter cultivars consistently yield higher than spring crops. The result would be an improvement of the economic viability and profitability of the crop. A further advantage would be the possibility to combine the growing of spring and winter crops, which would result in an extension of the harvest campaign by starting two to three months earlier, thus allowing for the improved capitalization on investments in equipment and infrastructure necessary for sugar beet harvesting, transport and processing.

In Arabidopsis thatiana functional analysis has distinguished four distinct flowering pathways (Levy and Dean, 1998). These four pathways can be assigned to environmental stimuli, such as photoperiodic and vernalization promotion pathways, or inherent developmental signals. e.g. autonomous promotion and floral repression pathways. In some species the timing of flowering is primarily influenced by environmental factors, such as photoperiod, light quality/quantity, vernalization and water or nutrient availability. Other species are influenced less by exogenous signals and rely more on endogenous ones, such as plant size or number of nodes.

One locus of interest is the FLOWERING LOCUS C (FLC) discovered in naturally occurring late-flowering ecotypes of Arabidopsis (Koornneef et al, 1994; Lee et al. 1994). FLC is a MADS box transcriptional regulator (Michaels, S D and R M Amasino, 1999) that represses flowering.

In contrast, there are genes that cause the switch from vegetative to reproductive growth, including the “flowering locus T” (FT), “leafy” (LFY), and “suppressor of over expression of constans” referred to as “Agamous-like 20” (AGL20). (Nilsson et al, 1998; Kobayashi et al., 1999; Blazquez et al., 2000; Lee et al, 2000; Samach et al., 2000; Borner et al., 2000) Overexpression of AGL20 causes early flowering in Arabidopsis, whereas its down-regulation causes late flowering.

In the case of sugar beet, it has been shown that the vernalization response is one of the most important factors of flower induction. Although bolting resistant varieties are known and available to sugar beet farmers, still there are major problems with the cultivation of the higher yielding winter beet due to bolting incidents. Currently, there are no plants or methods for predictably delaying sugar beet vernalization.

Vernalization and its effect on biennial sugar beet have been described in detail (e.g. Jaggard et al, 1983). Sugar beet responds to temperatures between 3 and 12° C. and cooling degrees accumulate. Several weeks of 3-12° C. are required for the beet to start bolting. The ITB Bolting Model shows that in France, vernalization occurs up to 90 days (13 weeks) after drilling. Seventeen days of 7° C. is the critical number during these 90 days to initiate bolting.

### SUMMARY OF THE INVENTION

The present invention includes sugar beet plants and methods for modulating sugar beet vernalization response by over expressing the FLC gene or by suppressing AGL20 gene expression in sugar beet.

In one embodiment, the invention relates to sugar beet plants and methods for modulating sugar beet vernalization response by overexpressing the FLC gene and by suppressing AGL20 gene expression in the same sugar beet plant.

### BRIEF DESCRIPTION OF THE FIGURES AND SEQ IDs

FIG. 1 is a plasmid map of binary vector pHiNK260.

FIG. 2 is an alignment of the cDNA sequences of the AGL20 homologues from Arabidopsis thaliana (AtAGL20), Nicotiana tabacum (NtAGL20) and Sinapsis alba (SaAGL20). The degenerate primers HiNK624 and HiNK619 were designed to the conserved regions and are shown underlined.

FIG. 3 is a phylogenetic tree inferred from the alignment of the coding region of the AGL20 homologue from sugar beet to the AGL20 homologues from Arabidopsis thaliana, Pinus taeda, Pisum sativum, Sinapsis alba and Nicotiana tabacum.

FIG. 4 is a plasmid map of binary vector pHiNK382.

FIG. 5 is a table containing the phenotypic results of FLC events.

FIG. 6 is a table containing the phenotypic results of AGL20 events.

FIG. 7 is a plasmid map of binary vector pHiNK440.

FIG. 8 is a plasmid map of binary vector pHiNK441.

FIG. 9 depicts an alignment of the three protein sequences for the Beta vulgaris FLC gene (BvFLC) that shows the INDELs discriminating between the three different splicing variants.

FIG. 10 depicts an alignment of the three splicing variants of the putative FLC homologue from sugar beet showing the two in-frame INDELs. The sequence of degenerate primer HiNK5279 that was used to amplify these three cDNA fragments is boxed.

FIG. 11 shows the result of the expression of the RNAi components of pHiNK 440 and 441, control gene GAPC and the endogenous sugar beet gene BvAGL20 by RT-PCR. The endogenous BvAGL20 gene was down regulated in the hybrid (A), but not in the plants, transgene for only one dsRNA, component (B and C), nor the NT (D). W=water; D=DNA; R=RNA; 0.5, 1 and 2=amount of cDNA in μl per RT-PCR reaction.

SEQ ID NO: 1 depicts the nucleotide sequence of binary vector pHiNK260 that carries an expression cassette comprising the Arabidopsis FLC gene.
SEQ ID NO: 2 depicts the nucleotide sequence of binary vector pHiNK382 that carries an expression cassette comprising an inverted repeat of the sugar beet AGL20 homologue.
SEQ ID NO: 3 depicts the nucleotide sequence of the Arabidopsis FLC cDNA (Accession No. AF537203)
SEQ ID NO: 4 depicts the nucleotide sequence of the partial genomic sequence of the sugar beet AGL20 homologue.
SEQ ID NO; 5 depicts the nucleotide sequence of a 0.28 Kb cDNA fragment consisting of exons 3 to 7 of the AGL20 homologue from sugar beet.
SEQ ID NO: 6 depicts the nucleotide sequence of the sugar beet AGL20 homolgue (BvAGL20).
SEQ ID NO: 7 depicts the nucleotide sequence of primer HiNK529.
SEQ ID NO: 8 depicts the nucleotide sequence of primer HiNK792
SEQ ID NO: 9 depicts the nucleotide sequence of primer HiNK793
SEQ ID NO: 10 depicts the nucleotide sequence of primer HiNK794
SEQ ID NO: 11 depicts the nucleotide sequence of primer HiNK795
SEQ ID NO: 12 depicts the nucleotide sequence of primer HiNK796
SEQ ID NO: 13 depicts the nucleotide sequence of primer HiNK624
SEQ ID NO: 14 depicts the nucleotide sequence of primer HiNK619
SEQ ID NO: 15 depicts the nucleotide sequence of primer HiNK725
SEQ ID NO: 16 depicts the nucleotide sequence of primer HiNK729
SEQ ID NO: 17 depicts the nucleotide sequence of primer HiNK2617
SEQ ID NO: 18 depicts the nucleotide sequence of primer HiNK2618
SEQ ID NO: 19 depicts the nucleotide sequence depicts the nucleotide sequence of binary vector pHiNK440 that carries an expression cassette comprising a fragment of the sugar beet AGL20 homologue in sense orientation.
SEQ ID NO: 20 depicts the nucleotide sequence depicts the nucleotide sequence of binary vector pHiNK441 that carries an expression cassette comprising a fragment of the sugar beet AGL20 homologue in antisense orientation.
SEQ ID NO: 21 depicts the nucleotide sequence of contig_{—}71+EST identifying the coding region of splicing variant 1 of the endogenous sugar beet FLC gene.
SEQ ID NO: 22 depicts the amino acid sequence of the expression product of the coding region of splicing variant 1 of the endogenous sugar beet FLC gene.
SEQ ID NO: 23 depicts the nucleotide sequence of contig_{—}78+EST identifying the coding region of splicing variant 2 of the endogenous sugar beet FLC gene.
SEQ ID NO: 24 depicts the amino acid sequence of the expression product of the coding region of splicing variant 2 of the endogenous sugar beet FLC gene
SEQ ID NO. 25 depicts the nucleotide sequence of contig_{—}79+EST identifying the coding region of splicing variant 3 of the endogenous sugar beet FLC gene.
SEQ ID NO: 26 depicts the amino acid sequence of the expression product of the coding region of splicing variant 3 of the endogenous sugar beet FLC gene.
SEQ ID NO: 27 depicts the nucleotide sequence of contig_{—}71+EST
SEQ ID NO: 28 depicts the nucleotide sequence of contig_{—}78+EST
SEQ ID NO: 29 depicts the nucleotide sequence of contig_{—}79+EST
SEQ ID NO: 30 depicts the nucleotide sequence of primer HiNK5277
SEQ ID NO: 31 depicts the nucleotide sequence of primer HiNK5279
SEQ ID NO: 32 depicts the nucleotide sequence of primer AGL20 A
SEQ ID NO: 33 depicts the nucleotide sequence of primer AGL20 B
SEQ ID NO: 34 depicts the nucleotide sequence of primer HiNK023
SEQ ID NO: 35 depicts the nucleotide sequence of primer gapCex5/6F
SEQ ID NO: 36 depicts the nucleotide sequence of primer gapCex8R
SEQ ID NO: 37 depicts the nucleotide sequence of primer HiNK HiNK 819

### DEFINITIONS

The technical terms and expressions used within the scope of this application are generally to be given the meaning commonly applied to them in the pertinent art of plant breeding and cultivation if not otherwise indicated herein below.

As used in this specification and the appended claims, the singular forms “a”, “an”, and “the” include plural referents unless the context clearly dictates otherwise. Thus, for example, reference to “a plant” includes one or more plants, and reference to “a cell” includes mixtures of cells, tissues, and the like.

“Sugar beet” refers to all species and subspecies within the genus Beta as well as all kinds of cultivated beets of Beta vulgaris. Cultivated beets have been separated into four groups: leaf beet, garden beet, fodder beet and sugar beet. “Sugar beet” refers also to all cultivated beets including those grown for other purposes than the production of sugar, such as ethanol, plastics or other industrial products. In particular, “Sugar beet” refers to fodder beet and sugar beet, but especially to sugar beet.

“Bolting” refers to the transition from the vegetative rosette stage to the inflorescence or reproductive growth stage.

“Vernalization” refers to the process by which floral induction in some plants is promoted by exposing the plants to chilling for a certain duration.

A “coding sequence” is a nucleic acid sequence that is transcribed into RNA such as mRNA, rRNA, tRNA, snRNA, sense RNA or antisense RNA. Preferably the RNA is then translated in an organism to produce a protein.

A “gene” is a defined region that is located within a genome and that, besides the aforementioned coding nucleic acid sequence, comprises other, primarily regulatory, nucleic acid sequences responsible for the control of the expression, that is to say the transcription and translation, of the coding portion. A gene may also comprise other 5′ and 3′ untranslated sequences and termination sequences. Further elements that may be present are, for example, introns.

The term “nucleic acid” refers to deoxyribonucleotides or ribonucleotides and polymers thereof in either single- or double-stranded form, composed of monomers (nucleotides) containing a sugar, phosphate and a base which is either a purine or pyrimidine. Unless specifically limited, the term encompasses nucleic acids containing known analogs of natural nucleotides which have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions) and complementary sequences as well as the sequence explicitly indicated. Specifically, degenerate codon substitutions may be achieved by generating sequences in which the third position of one or more selected (or all) codons is substituted with mixed-base and/or deoxyinosine residues. A “nucleic acid fragment” is a fraction of a given nucleic acid molecule. In higher plants, deoxyribonucleic acid (DNA) is the genetic material while ribonucleic acid (RNA) is involved in the transfer of information contained within DNA into proteins. The term “nucleotide sequence” refers to a polymer of DNA or RNA which can be single- or double-stranded, optionally containing synthetic, non-natural or altered nucleotide bases capable of incorporation into DNA or RNA polymers. The terms “nucleic acid” or “nucleic acid sequence” may also be used interchangeably with gene, cDNA, DNA and RNA encoded by a gene.

The term “heterologous” when used in reference to a gene or nucleic acid refers to a gene encoding a factor that is not in its natural environment (i.e., has been altered by the hand of man). For example, a heterologous gene may include a gene from one species introduced into another species. A heterologous gene may also include a gene native to an organism that has been altered in some way (e.g., mutated, added in multiple copies, linked to a non-native promoter or enhancer sequence, etc.). Heterologous genes further may comprise plant gene sequences that comprise cDNA forms of a plant gene; the cDNA sequences may be expressed in either a sense (to produce mRNA) or anti-sense orientation (to produce an anti-sense RNA transcript that is complementary to the mRNA transcript). In one aspect of the invention, heterologous genes are distinguished from endogenous plant genes in that the heterologous gene sequences are typically joined to nucleotide sequences comprising regulatory elements such as promoters that are not found naturally associated with the gene for the protein encoded by the heterologous gene or with plant gene sequences in the chromosome, or are associated with portions of the chromosome not found in nature (e.g., genes expressed in loci where the gene is not normally expressed).

“Inverted repeat” refers to a nucleotide sequence found at two sites on the same nucleic acid sequence, but in opposite orientation.

“Expression cassette” as used herein means a nucleic acid molecule capable of directing expression of a particular nucleotide sequence or sequences in an appropriate host cell, comprising a promoter operably linked to the nucleotide sequence or sequences of interest which is/are operably linked to termination signals. It also typically comprises sequences required for proper translation of the nucleotide sequencers). The expression cassette may also comprise sequences not necessary in the direct expression of a nucleotide sequence of interest but which are present due to convenient restriction sites for removal of the cassette from an expression vector. The expression cassette comprising the nucleotide sequence(s) of interest may be chimeric, meaning that at least one of its components is heterologous with respect to at least one of its other components. The expression cassette may also be one that is naturally occurring but has been obtained in a recombinant form useful for heterologous expression. Typically, however, the expression cassette is heterologous with respect to the host, i.e., the particular nucleic acid sequence of the expression cassette does not occur naturally in the host cell and must have been introduced into the host cell or an ancestor of the host cell by a transformation process known in the art. The expression of the nucleotide sequence(s) in the expression cassette may be under the control of a constitutive promoter or of an inducible promoter that initiates transcription only when the host cell is exposed to some particular stimulus, which may be an external stimulus or an internal stimulus being provided from the host itself. In the case of a multicellular organism, such as a plant, the promoter can also be specific to a particular tissue, or organ, or stage of development. An expression cassette, or fragment thereof can also be referred to as “inserted sequence” or “insertion sequence” when transformed into a plant.

“Operably-linked” refers to the association of nucleic acid sequences on a single nucleic acid fragment so that the function of one affects the function of the other. For example, a promoter is operably-linked with a coding sequence or functional RNA when it is capable of affecting the expression of that coding sequence or functional RNA (i.e., that the coding sequence or functional RNA is under the transcriptional control of the promoter). Coding sequences in sense or antisense orientation can be operably-linked to regulatory sequences.

“Primers” as used herein are isolated nucleic acids that are capable of becoming annealed to a complimentary target DNA strand by nucleic acid hybridization to form a hybrid between the primer and the target DNA strand, then extended along the target DNA strand by a polymerase, such as DNA polymerase. Primer pairs or sets can be used for amplification of a nucleic acid molecule, for example, by the polymerase chain reaction (PCR) or other conventional nucleic-acid amplification methods.

“Suppression” refers to the absence or observable decrease in the level of protein and/or mRNA product from a target gene. In particular, “suppression” refers to a decrease in the level of protein and/or mRNA product from a target gene in the range of between 20% and 100%, particularly of between 40% and 80%, more particularly of between 50% and 90%, even more particularly of between 60% and 95%, but especially of between 75% and 98% and up to 100%. The consequences of inhibition can be confirmed by examination of the outward properties of the cell or organism or by biochemical and gene expression detection techniques known to those skilled in the art. For example, suppression of the AGL20 gene expression is indicated by an absence or delay of the vernalization response in a growing sugar beet plant.

Substantially identical or homologous in the context of two nucleic acid or protein sequences, refers to two or more sequences or subsequences that have at least 60%, preferably 80%, more preferably 90%, even more preferably 95%, and most preferably at least 99% nucleotide or amino acid residue identity, when compared and aligned for maximum correspondence, as measured using one of the following sequence comparison algorithms or by visual inspection. In particular, the substantial identity exists over a region of the sequences that is at least about 50 residues in length, more particularly over a region of at least about 100 residues, and especially the sequences are substantially identical over at least about 150 residues. In a specific embodiment, the sequences are substantially identical over the entire length of the coding regions. Furthermore, substantially identical nucleic acid or protein sequences perform substantially the same function.

For sequence comparison, typically one sequence acts as a reference sequence to which test sequences are compared. When using a sequence comparison algorithm, test and reference sequences are input into a computer, subsequence coordinates are designated if necessary, and sequence algorithm program parameters are designated. The sequence comparison algorithm then calculates the percent sequence identity for the test sequencers) relative to the reference sequence, based on the designated program parameters.

Optimal alignment of sequences for comparison can be conducted, e.g., by the local homology algorithm of Smith & Waterman (1981), by the homology alignment algorithm of Needleman & Wunsch (1970), by the search for similarity method of Pearson & Lipman, Proc. Nat'l. Acad. Sci. USA 85: 2444 (1988), by computerized implementations of these algorithms (GAP, BESTFIT, FASTA, and TFASTA in the Wisconsin, Genetics Software Package, Genetics Computer Group, 575 Science Dr., Madison, Wis.), or by visual inspection (see generally, Ausubel et al., infra). One example of an algorithm that is suitable for determining percent sequence identity and sequence similarity is the BLAST algorithm, which is described in Altschul et al. (1990).

Another indication that two nucleic acid sequences are substantially identical is that the two molecules hybridize to each other under stringent conditions. The phrase “hybridizing specifically to” refers to the binding, duplexing, or hybridizing of a molecule only to a particular nucleotide sequence under stringent conditions when that sequence is present in a complex mixture (e.g., total cellular) DNA or RNA “Bind(s) substantially” refers to complementary hybridization between a probe nucleic acid and a target nucleic acid and embraces minor mismatches that can be accommodated by reducing the stringency of the hybridization media to achieve the desired detection of the target nucleic acid sequence.

“Stringent hybridization conditions” and “stringent hybridization wash conditions” in the context of nucleic acid hybridization experiments such as Southern and Northern hybridizations are sequence dependent, and are different under different environmental parameters. Longer sequences hybridize specifically at higher temperatures. An extensive guide to the hybridization of nucleic acids is found in Tijssen (1993).

A further indication that two nucleic acid sequences or proteins are substantially identical is that the protein encoded by the first nucleic acid is immunologically cross reactive with, or specifically binds to, the protein encoded by the send nucleic acid. Thus, a protein is typically substantially identical to a second protein, for example, where the two proteins differ only by conservative substitutions.

“Synthetic” refers to a nucleotide sequence comprising structural characters that are not present in the natural sequence. For example, an artificial sequence that resembles more closely the 3+C content and the normal codon distribution of dicot and/or monocot genes is said to be synthetic.

“Transformation” is a process for introducing heterologous nucleic acid into a host cell or organism. In particular, “transformation” means the stable integration of a DNA molecule into the genome of an organism of interest.

“Transformed/transgenic/recombinant” refers to a host organism such as a bacterium or a plant into which a heterologous nucleic acid molecule has been introduced. The nucleic acid molecule can be stably integrated into the genome of the host or the nucleic acid molecule can also be present as an extrachromosomal molecule. Such an extrachromosomal molecule can be auto-replicating. Transformed cells, tissues, or plants are understood to encompass not only the end product of a transformation process, but also transgenic progeny thereof. A “non-transformed”, “non-transgenic”, or “non-recombinant” host refers to a wild-type organism, e.g., a bacterium or plant, which does not contain the heterologous nucleic acid molecule.

The term transgenic “event” refers to a recombinant plant produced by transformation and regeneration of a single plant cell with heterologous DNA, for example, an expression cassette that includes a gene of interest. The term “event” refers to the original transformant and/or progeny of the transformant that include the heterologous DNA. The term “event” also refers to progeny produced by a sexual outcross between the transformant and another sugar beet line. Even after repeated backcrossing to a recurrent parent, the inserted DNA and the flanking DNA from the transformed parent is present in the progeny of the cross at the same chromosomal location. Normally, transformation of plant tissue produces multiple events, each of which represent insertion of a DNA construct into a different location in the genome of a plant cell. Based on the expression of the transgene or other desirable characteristics, a particular event is selected.

A “transgene” refers to a gene introduced into the genome of an organism by genetic manipulation in order to alter its genotype.

A “transgenic plant” is a plant having one or more plant cells that contain an expression vector.

The term “Messenger RNA (mRNA) refers to the RNA that is without introns and that can be translated into protein by the cell.

“cDNA” refers to a single- or a double-stranded DNA that is complementary to and derived from mRNA.

The term “expression” when used in reference to a nucleic acid sequence, such as a gene, refers to the process of converting genetic information encoded in a gene into RNA (e.g., mRNA, rRNA, tRNA, or snRNA) through “transcription” of the gene (i.e., via the enzymatic action of an RNA polymerase), and into protein where applicable (as when a gene encodes a protein), through “translation” of mRNA. Gene expression can be regulated at many stages in the process.

“Overexpression” refers to the level of expression in transgenic cells or organisms that exceeds levels of expression in normal or untransformed (nontransgenic) cells or organisms.

“Antisense inhibition” refers to the production of antisense RNA transcripts capable of suppressing the expression of protein from an endogenous gene or a transgene.

“Gene silencing” refers to homology-dependent suppression of viral genes, transgenes, or endogenous nuclear genes. Gene silencing may be transcriptional, when the suppression is due to decreased transcription of the affected genes, or post-transcriptional, when the suppression is due to increased turnover (degradation) of RNA species homologous to the affected genes. Gene silencing includes virus-induced gene silencing.

“RNA interference” (RNAi) refers to the process of sequence-specific post-transcriptional gene silencing in plants and animals mediated by short interfering RNAs (siRNAs). Various terms such as siRNA, target RNA molecule, dicer or ribonuclease III enzyme are concepts known to those skilled in the art and full descriptions of these terms and other concepts pertinent to RNAi can be found in the literature. For reference, several terms pertinent to RNAi are defined below. However, it is understood that any particular hypothesis describing the mechanisms of RNAi are not necessary to practice the present invention.

The term “siRNAs” refers to short interfering RNAs. In some embodiments, siRNAs comprise a duplex, or double-stranded region, of about 21-23 nucleotides long; often siRNAs contain from about two to four unpaired nucleotides at the 3′ end of each strand. At least one strand of the duplex or double-stranded region of a siRNA is substantially homologous to or substantially complementary to a target RNA molecule. The strand complementary to a target RNA molecule is the “antisense strand;” the strand homologous to the target RNA molecule is the “sense strand,” and is also complementary to the siRNA antisense strand. siRNAs may also contain additional sequences; non-limiting examples of such sequences include linking sequences, or loops, as well as stem and other folded structures. siRNAs appear to function as key intermediaries in triggering RNA interference in invertebrates and in vertebrates, and in triggering sequence-specific RNA degradation during posttranscriptional gene silencing in plants.

The term “target RNA molecule” refers to an RNA molecule to which at least one strand of the short double-stranded region of a siRNA is homologous or complementary. Typically, when such homology or complementary is about 100%, the siRNA is able to silence or inhibit expression of the target RNA molecule. Although it is believed that processed mRNA is a target of siRNA, the present invention is not limited to any particular hypothesis, and such hypotheses are not necessary to practice the present invention. Thus, it is contemplated that other RNA molecules may also be targets of siRNA. Such RNA target molecules include unprocessed mRNA, ribosomal RNA, and viral RNA genomes.

“RNA-inducing silencing complex” (RISC) mediates cleavage of single-stranded RNA having sequence complementary to the antisense strands of siRNA duplex. Cleavage of the target RNA takes place in the middle of the region of complementary to the antisense strand of the siRNA duplex (Elbashier et al. 2001).

The term “sufficient complementary” means that a first or a second strand sequence of RNA introduced into a plant cell is capable of hybridizing or annealing sufficiently to the RNA produced by a target gene (mRNA) under conditions found in the cytoplasm of said plant cell, such that suppression of the expressions of the target gene is triggered. For example, the strand of the first or second strand sequence of RNA that binds to the mRNA produced by the target gene is at least 50% identical to the corresponding mRNA sequence of the target gene, more desirably at least 70% identical, yet more desirable is at least 90% identity and even more desirable is at least 95% identical.

It is to be understood that the percentage of identity between the strand of the first or second strand sequence of RNA and the mRNA produced by the target gene, which is in the range of between at least 70% identity and at least 95% identity, can be any numerical value within this range.

### DETAILED DESCRIPTION OF THE INVENTION

The present invention includes transgenic sugar beet plants and methods for modulating sugar beet vernalization response by over expressing an FLC gene and/or by suppressing AGL20 gene expression in sugar beet.

One embodiment of the invention includes constitutively expressing an FLC gene resulting in modulation of bolting resistance in sugar beet. According to the present invention, transgenic sugar beet plants overexpressing a FLC gene no longer respond to a typical vernalization period of 18 weeks by bolting and subsequent flowering, but to the contrary continue vegetative growth (non-bolting) and develop a normal taproot.

The invention includes a transgenic sugar beet plant comprising in its genome the coding region of a heterologous FLC gene, wherein expression of the FLC gene causes overexpression of the FLC gene product thereby suppressing the vernalization response of the sugar beet plant. A gene is considered to be overexpressed if the expression rate is above the basic level of expression normally found in the native, untransformed sugar beet plant. In particular, overexpression refers to an expression rate which exceeds the basic level of expression normally found in the native, untransformed sugar beet plant by at least 10%, particularly by at least 20%, more particularly by at least 30%, even more particularly by at least 40%, but especially by at least 50% to 100% or higher.

In a specific embodiment of the invention, a sugar beet plant is, provided wherein said heterologous FLC gene comprises the heterologous FLC coding region consisting of the FLC cDNA depicted under Accession No, AF537203 (http://ftp.dna.affrc.go.jp-/pub/dna_all/A/F5/37/20/AF537203/AF537203), particularly under the control of a heterologous constitutive promoter causing overexpression of the FLC gene product thereby suppressing the vernalization response of the sugar beet plant.

The present invention also includes a transgenic sugar beet plant comprising a heterologous FLC gene as depicted by SEQ ID NO: 3, wherein expression of the FLC gene causes overexpression of the FLC gene product thereby suppressing the vernalization response of the sugar beet plant.

The present invention also includes a transgenic sugar beet plant comprising an endogenous FLC gene as depicted in SEQ ID NOs: 27, 28 and 29 or the encoding part thereof, particularly under the control of a heterologous constitutive promoter causing overexpression of the endogenous gene product thereby suppressing the vernalization response of the sugar beet plant.

The present invention also includes a transgenic sugar beet plant comprising an endogenous FLC gene as depicted in SEQ ID NOs: 21, 23 and 25 or the encoding part thereof, particularly under the control of a heterologous constitutive promoter causing overexpression of the endogenous gene product thereby suppressing the vernalization response of the sugar beet plant.

The invention also includes a transgenic sugar beet plant comprising a heterologous FLC gene that has at least 99%, 98%, 97%, 96%, 95%, 94% or 93% sequence identity with the nucleotide sequence of the FLC cDNA depicted under Accession No. AF537203, particularly under the control of a heterologous constitutive promoter causing overexpression of the FLC gene product thereby suppressing the vernalization response of the sugar beet plant.

In a specific embodiment, a heterologous FLC gene is provided that has at least 99%, 98%, 97%, 96%, 95%, 94% or 93% sequence identity with the nucleotide sequence of the FLC cDNA as depicted by SEQ ID NO: 3, wherein expression of the FLC gene causes overexpression of the FLC gene product thereby suppressing the vernalization response of the sugar beet plant.

The invention also includes a transgenic sugar beet plant comprising an endogenous FLC gene that has at least 99%, 98%, 97%, 96%, 95%, 94% or 93% sequence identity with the nucleotide sequence depicted in SEQ ID NOs: 27, 28 and 29 or the encoding part thereof, which encoding part may be under the control of a heterologous constitutive promoter, wherein expression of the FLC gene causes overexpression of the FLC gene product thereby suppressing the vernalization response of the sugar beet plant.

The invention also includes a transgenic sugar beet plant comprising an endogenous FLC gene that has at least 99%, 98%, 97%, 96%, 95%, 94% or 93% sequence identity with the nucleotide sequence depicted in SEQ ID NOs: 21, 23 and 25 or the encoding part thereof, which encoding part may be under the control of a heterologous constitutive promoter, wherein expression of the FLC gene causes overexpression of the FLC gene product thereby suppressing the vernalization response of the sugar beet plant.

The invention also includes a seed of the transgenic sugar beet plant comprising a heterologous FLC gene comprising a coding region consisting of the FLC cDNA depicted under Accession No. AF537203, particularly under the control of a heterologous constitutive promoter causing overexpression of the FLC gene product thereby suppressing the vernalization response of the sugar beet plant.

In a specific embodiment, a heterologous FLC gene is provided as depicted by SEQ ID NO: 3, wherein expression of the FLC gene in a plant grown form said seed causes overexpression of the FLC gene product thereby suppressing the vernalization response of the sugar beet plant.

The invention also includes a seed of the transgenic sugar beet plant comprising an endogenous FLC gene as depicted in SEQ ID NOs: 27, 28 and 29 or the encoding part thereof, particularly the encoding part of an endogenous FLC gene under the control of a heterologous constitutive promoter, wherein expression of the FLC gene in a plant grown form said seed causes overexpression of the FLC gene product thereby suppressing the vernalization response of the sugar beet plant.

The invention also includes a seed of the transgenic sugar beet plant comprising an endogenous FLC gene as depicted in SEQ ID NOs: 21, 23 and 25 or the encoding part thereof, particularly the encoding part of an endogenous FLC gene under the control of a heterologous constitutive promoter, wherein expression of the FLC gene in a plant grown form said seed causes overexpression of the FLC gene product thereby suppressing the vernalization response of the sugar beet plant.

The invention also includes a seed of a transgenic sugar beet plant, wherein the seed comprises a heterologous FLC gene that has at least 99%, 98%, 97%, 96%, 95%, 94% or 93% sequence identity with the nucleotide sequence of the FLC cDNA depicted under Accession No. AF537203, particularly under the control of a heterologous constitutive promoter causing overexpression of the FLC gene product thereby suppressing the vernalization response of the sugar beet plant.

In a specific embodiment, a heterologous FLC gene is provided that has at least 99%, 98%, 97%, 96%, 95%, 94% or 93% sequence identity with the nucleotide sequence of the FLC cDNA as depicted by SEQ ID NO: 3, wherein expression of the FLC gene in a plant grown form said seed causes overexpression of the FLC gene product thereby suppressing the vernalization response of the sugar beet plant.

The invention also includes a seed of a transgenic sugar beet plant, wherein the seed comprises an endogenous FLC gene that has at least 99%, 98%, 97%, 96%, 95%, 94% or 93% sequence identity with the nucleotide sequence depicted in SEQ ID NOs: 27, 28 and 29 or with the encoding part thereof, which encoding part may be under the control of a heterologous constitutive promoter, wherein expression of the FLC gene in a plant grown form said seed causes overexpression of the FLC gene product thereby suppressing the vernalization response of the sugar beet plant.

The invention also includes a seed of a transgenic sugar beet plant, wherein the seed comprises an endogenous FLC gene that has at least: 99%, 98%, 97%, 96%, 95%, 94% or 93% sequence identity with the nucleotide sequence depicted in SEQ ID NOs: 21, 23 and 25 or with the encoding part thereof, which encoding part may be under the control of a heterologous constitutive promoter, wherein expression of the FLC gene in a plant grown form said seed causes overexpression of the FLC gene product thereby suppressing the vernalization response of the sugar beet plant.

One embodiment of the invention includes a transgenic sugar beet plant according to the invention comprising a heterologous FLC gene as disclosed herein before incorporated in an expression cassette, particularly an expression cassette depicted by nucleotide sequence 786-2817 of SEQ ID NO: 1.

One embodiment of the invention includes a transgenic sugar beet plant according to the invention comprising a heterologous FLC gene as disclosed herein before incorporated in an expression cassette, particularly an expression cassette depicted by nucleotide sequence 786-2817 of SEQ ID NO: 1, wherein the expression cassette comprises a constitutive promoter.

One embodiment of the invention includes a transgenic sugar beet plant according to the invention comprising a heterologous FLC gene as disclosed herein before incorporated in an expression cassette, particularly an expression cassette depicted by nucleotide sequence 786-2817 of SEQ ID NO: 1, wherein the expression cassette comprises a CaMV35S promoter.

In another specific embodiment, the invention relates to a transgenic sugar beet plant comprising a heterologous FLC gene as disclosed herein before, particularly incorporated in an expression cassette under the control of a constitutive promoter, particularly the constitutive CaMV 35S promoter and a terminator, particularly the mannopine synthase (mas) terminator from Agrobacterium tumefaciens.

In one embodiment, the invention relates to a transgenic sugar beet plant comprising an expression cassette comprising a coding region of an endogenous FLC gene, particularly an FLC gene as depicted in SEQ ID NOs: 27, 28 and 29.

In one embodiment, the invention relates to a transgenic sugar beet plant comprising an expression cassette comprising a coding region of an endogenous FLC gene, particularly an FLC gene as depicted in SEQ ID NOs: 21, 23 and 25.

In a specific embodiment, said coding region of the endogenous FLC gene is under the control of a constitutive promoter, particularly under the control of a constitutive CaMV 35S promoter, but especially under the control of the constitutive CaMV 35S promoter and a terminator, particularly the mannopine synthase (mas) terminator from Agrobacterium tumefaciens.

Another embodiment of the invention is a method of producing a transgenic sugar beet plant according to the invention comprising:
transforming a sugar beet plant cell with a transgene comprising regulatory sequences operably linked to a heterologous or an endogenous plant FLC gene coding region;
identifying a sugar beet plant cell carrying the inserted transgene; and
regenerating a transgenic plant from the plant cell identified in b).

The present invention further includes producing biofuels, such as ethanol, butanol, methanol, biogas and diesel derived from a transgenic sugar beet plant according to the invention and as described herein before comprising in its genome the coding region of a heterologous or of an endogenous FLC gene, wherein expression of said FLC gene causes over expression of the FLC gene product thereby suppressing the vernalization response of said sugar beet plan.

The present invention further includes producing other industrial applications such as plastics derived from a transgenic sugar beet plant according to the invention and as described herein before comprising in its genome the coding region of a heterologous or of an endogenous FLC gene, wherein expression of said FLC gene causes over expression of the FLC gene product thereby suppressing the vernalization response of said sugar beet plant.

The present invention also includes a method of suppressing the expression of an endogenous AGL20 gene of a sugar beet plant cell, comprising introducing into said plant cell a first RNA strand and; a second RNA strand, wherein said first RNA strand or, in the alternative, said second strand is sufficiently complimentary to at least a portion of an RNA strand of said endogenous AGL20 gene to hybridize or anneal to the RNA produced by the AGL20 gene such as to cause suppression of the expression of the endogenous AGL20 gene and said first RNA strand and said second RNA strand form a double stranded RNA, wherein said double stranded RNA participates in RNA interference of expression of said endogenous AGL20 gene.

One embodiment of the invention includes a method of suppressing the expression of an endogenous AGL20 gene of a sugar beet plant cell comprising introducing into said plant cell a first RNA strand and a second RNA strand, wherein introducing into said plant cell a first RNA strand and a second RNA strand comprises transforming said cell with an heterologous DNA, which when transcribed in the plant cell, yields a nucleotide sequence corresponding to said first RNA strand and a nucleotide sequence corresponding to said second RNA strand.

In another embodiment of the invention said heterologous DNA includes an inverted repeat, which when transcribed, yields a nucleotide sequence corresponding to said first RNA strand and a nucleotide sequence corresponding to said second RNA strand.

In a specific embodiment, the invention relates to a method of suppressing the expression of an endogenous AGL20 gene according to the invention and as described herein before, wherein said first RNA strand has a degree of complementarity to a portion of RNA of a sugar beet AGL20 gene fragment approximately 0.6 Kb in size obtainable from sugar beet cDNA obtained from total RNA extracted from sugar beet leaves in a reverse trascriptase reaction using primer 5′-CCRATGAACARTTSNGTCTCNACWTC-3′ (SEQ ID NO: 14), which cDNA is used as a template in a PCR reaction employing a degenerate forward primer with the nucleotide sequence 5′-ATGGTKMGRGGNAARACNCAGATGA-3′ (SEQ ID NO: 13), which shares sequence homology to the extreme NH2-terminus starting at the ATG codon and spanning codons 1 to 9; and a degenerate reverse primer with the nucleotide sequence 5′-CCRATGAACARTTSNGTCTCNACWTC-3′ (SEQ ID NO: 14), which is complementary to the COOH-terminus, hybridizing just upstream of the stop codon at exon 8, such as to allow said first RNA strand to hybridize or anneal to the RNA strand of said AGL20 gene fragment resulting in the suppression of the expression of the endogenous AGL20 gene.

Yet another embodiment of the invention includes the method of suppressing the expression of an endogenous AGL20 gene as described herein before, wherein said first RNA strand is sufficiently complimentary to a portion of RNA of the sugar beet AGL20 gene fragment depicted in SEQ ID NO: 6 to hybridize or anneal to the RNA produced by the AGL20 gene such as to cause suppression of the expression of the endogenous AGL20 gene.

This suppression of the AGL20 gene leads to a delay of the vernalization response in a growing sugar beet plant or causes the sugar beet plant to develop a non-bolting phenotype, which means that the sugar beet plant does no longer respond to a typical vernalization period of 18 weeks by bolting and subsequent flowering, but to the contrary continue vegetative growth (non-bolting) and develop a normal taproot.

Plants expressing said delayed vernalisation response or said non-bolting phenotype can be easily identified and selected by applying a phenotypic analysis experiment employing standardized growth conditions.

The invention also includes the method of suppressing the expression of an endogenous AGL20 gene as described herein before, wherein said first RNA strand comprises a sequence fragment about 21 to about 23 nucleotides in length that is sufficiently complementary to a portion of RNA of said sugar beet AGL20 gene such as to cause suppression of the expression of the endogenous AGL20 gene.

The invention includes the method of suppressing the expression of an endogenous AGL20 gene as described herein before, wherein said first RNA strand comprises a sequence fragment about 21 to about 25 nucleotides in length that is sufficiently complementary to a portion of RNA of said sugar beet AGL20 gene such as to cause suppression of the expression of the endogenous AGL20 gene.

The invention further includes the method of suppressing the expression of an endogenous AGL20 gene according to the invention, Wherein said first RNA strand comprises a sequence fragment about 21 to about 30 nucleotides in length that is sufficiently complementary to a portion of RNA of said sugar beet AGL20 gene such as to cause suppression of the expression of the endogenous AGL20 gene.

One embodiment of the invention further includes the method of suppressing the expression of an endogenous AGL20 gene according to the invention, wherein said first RNA, strand comprises a sequence fragment about 18 to about 23 nucleotides in length that is sufficiently complementary to a portion of RNA of said sugar beet AGL20 gene to result in the suppression of the expression of the endogenous AGL20 gene.

One embodiment of the invention further includes the method of suppressing the expression of an endogenous AGL20 gene according to the invention, wherein said first RNA strand comprises a sequence fragment about 18 through 25 nucleotides in length that is sufficiently complementary to a portion of RNA of said sugar beet AGL20 gene to result in the suppression of the expression of the endogenous AGL20 gene.

One embodiment of the invention further includes the method of suppressing the expression of an endogenous AGL20 gene according to the invention, wherein said first RNA strand comprises a sequence fragment about 18 through 30 nucleotides in length that is sufficiently to a portion of RNA of said sugar beet AGL20 gene to result in the suppression of the expression of the endogenous AGL20 gene.

In one embodiment, the invention relates to a method of suppressing expression of an AGL20 gene according to the invention and as described herein before, wherein the heterologous DNA that transcribes said first RNA strand is obtainable from a 0.28 Kb cDNA fragment consisting of exons 3 to 7 of the AGL20 gene fragment approximately 0.6 Kb in size obtainable from sugar beet cDNA obtained from total RNA extracted from sugar beet leaves in a reverse trascriptase reaction using primer 5′-CCRATGAACARTTSNGTCTCNACWTC-3′ (SEQ ID NO: 14), which cDNA is used as a template in a PCR reaction employing a degenerate forward primer with the nucleotide sequence 5′-ATGGTKMGRGGNAARACNCAGATGA-3′ (SEQ ID NO: 13), which shares sequence homology to the extreme NH2-terminus starting at the ATG codon and spanning codons 1 to 9; and a degenerate reverse primer with the nucleotide sequence 5′-CCRATGAACARTTSNGTCTCNACWTC-3′ (SEQ ID NO: 14), which is complementary to the COOH-terminus, hybridizing just upstream of the stop codon at exon 8, in a PCR reaction using a forward primer with the nucleotide sequence 5′-CTATGGATCCGCATGCTG ATCTCCTGATC-3′ (SEQ ID NO: 8) and a reverse primer with the nucleotide sequence 5′- [symbol] [symbol] [symbol] AAGA AGTTAAAAAGTCTCGAAC-3′ (SEQ ID NO: 9).

One embodiment of the invention further includes the method of suppressing expression of an AGL20 gene, wherein the heterologous DNA that transcribes said first RNA strand is depicted by SEQ ID NO: 5.

In one aspect, the invention relates to an expression cassette comprising a heterologous DNA comprising a first RNA strand and a second RNA strand wherein said first RNA strand has a degree of complementarity to at least a portion of an RNA strand of an endogenous AGL20 gene which allows said first RNA strand to hybridize or anneal to the RNA strand of said endogenous AGL20 gene and wherein said first RNA strand and said second RNA strand form a double stranded RNA such that upon expression in a plant suppression of the endogenous AGL20 gene is caused.

In a specific embodiment of the invention, an expression cassette is provided comprising an inverted repeat, which, when transcribed in the sugar beet cell, forms a double stranded RNA molecule in said plant cell comprising said first and second RNA strands.

In another specific embodiment of the inventions, an expression cassette is provided, wherein said inverted repeat is operatively linked to a constitutive promoter, particularly a CaMV promoter.

In one embodiment, the invention relates to an expression cassette as described herein before comprising a heterologous DNA that transcribes said first RNA strand, which heterologous DNA is obtainable from a 0.28 Kb cDNA fragment consisting of exons 3 to 7 of an AGL20 gene fragment approximately 0.6 Kb in size obtainable from sugar beet cDNA obtained from total RNA extracted from sugar beet leaves in a reverse trascriptase reaction using primer 5′-CCRATGAACARTTSNGTCTCNACWTC-3′ (SEQ ID NO: 14), which cDNA is used as a template in a PCR reaction employing a degenerate forward primer with the nucleotide sequence 5′-ATGGTKMGRGGNAARACNCAGATGA-3′ (SEQ ID NO: 13), which shares sequence homology to the extreme NH2-terminus starting at the ATG codon and spanning codons 1 to 9; and a degenerate reverse primer with the nucleotide sequence 5′-CCRATGAACARTTSNGTCTCNACWTC-3′ (SEQ ID NO: 14), which is complementary to the COOH-terminus, hybridizing just upstream of the stop codon at exon 8, in a PCR reaction using a forward primer with the nucleotide sequence 5′-CTATGGATCCGCATGCTG ATCTCCTGATC-3′ and a reverse primer with the nucleotide sequence 5′- [symbol] [symbol] [symbol] AAGA AGTTAAAAAGTCTCGAAC-3′.

In another specific embodiment of the invention the expression cassette comprises a heterologous DNA as depicted by SEQ ID NO: 5.

In one embodiment an expression cassette according to the invention and as described herein above comprises an inverted repeat, which, when transcribed in the sugar beet cell, forms a double stranded RNA molecule in said plant cell comprising said first and second RNA strands.

In a specific embodiments said inverted repeat is operatively linked to a constitutive promoter, particularly a CaMV promoter.

In one embodiment, the invention relates to an expression cassette as described herein before, wherein said heterologous DNA is inserted between a promoter and terminator which heterologous DNA is obtainable by
a) amplifying a 0.28 Kb cDNA fragment consisting of exons 3 to 7 of the 0.6 Kb AGL20 gene fragment according to the invention and as described herein before, in a recombinant PCR reaction using a forward primer with the nucleotide sequence 5′-CTATGGATCCGCATGCTG ATCTCCTGATC-3′ (SEQ ID NO: 8) and a reverse primer with the nucleotide sequence 5′- [symbol] [symbol] [symbol] AAGA AGTTAAAAAGTCTCGAAC-3′ (SEQ ID NO: 9);
b) amplifying a 0.19 Kb fragment comprising the ST-LS1 intron and flanking splicing sites using forward primer 5′-ATCCAACCGCGGACCTGCACATCAACAA-3′ (SEQ ID NO: 7) and reverse primer 5′- [symbol] [symbol] [symbol] [symbol] AGGTAAGTTTCTGCTTCTAC-3 (SEQ ID NO: 12);
c) fusing the amplification products obtained in steps a) and b) to each other by means of a second round of PCR using primers of SEQ ID NO:8 and SEQ ID NO:7 and using a mix of both amplification products as template, yielding a fusion product of 0.47 Kb in length;
d) amplifying the 0.28 Kb BvAGL20 fragment a second time, using forward primer 5′-TAAATCCGCGGAAGAAGTTAAAAAGTCTCGAAC-3′ (SEQ ID NO: 10) and reverse primer 5′-CTATTTGTCGACGCATGCTGATCTCCTGATC-3′ (SEQ ID NO: 11) that differ from the primer used in step a) with respect to their linkers;
e) fusing both fragments at the Sac II restriction sites to create an inverted repeat for the BvAGL20 sequence separated by the intron from the potato ST-LS1 gene.

In a specific embodiment, an expression cassette is provided as depicted by the nucleotide sequence 233-2657 of SEQ ID NO: 2.

One embodiment of the invention further includes the method of suppressing the expression of an endogenous AGL20 gene according to the invention, wherein introducing said first and second RNA strands is by insertion of an expression cassette according to the invention and as described herein before comprising said heterologous DNA into the genome of said plant cell.

One embodiment of the invention further includes the method of suppressing the expression of an endogenous AGL20 gene according to the invention, wherein said expression cassette is depicted by the nucleotide sequence 233-2657 of SEQ ID NO: 2.

One embodiment of the invention further includes the method of suppressing the expression of an endogenous AGL20 gene according to the invention, wherein introducing said first and second RNA strands is by insertion of said strands into the plant cell by injection.

One embodiment of the invention further includes the method of suppressing the expression of an endogenous AGL20 gene according to the invention, further comprising introducing into the genome of the plant an expression cassette that includes an inverted repeat, which when transcribed, forms a double stranded RNA molecule in said plant cell comprising said first and second RNA strands.

One embodiment of the invention further includes the method of suppressing the expression of an endogenous AGL20 gene according to the invention, wherein said first RNA sequence is sufficiently complementary to an RNA sequence of the nucleic acid sequence depicted by SEQ ID NO: 6.

The invention further includes the method of suppressing the expression of an endogenous AGL20 gene according to the invention, wherein said inverted repeat is operatively linked to a constitutive promoter.

The invention includes the method of suppressing the expression of an endogenous AGL20 gene according to the invention, further comprising an intron located between said first and second RNA strands.

The invention also includes the method of suppressing the expression of an endogenous AGL20 gene according to the invention, wherein said intron is depicted by the nucleotide sequence depicted by 817-1009 of SEQ ID NO: 2.

The invention includes a transgenic sugar beet cell, particularly a transgenic sugar beet plant, comprising a heterologous gene construct, said construct comprising a heterologous DNA, which when transcribed in the sugar beet cell, yields a first RNA nucleotide sequence and a second RNA nucleotide sequence, wherein said first RNA nucleotide sequence is sufficiently complimentary to at least a portion of a RNA strand of said endogenous AGL20 gene to hybridize or anneal to the RNA produced by the AGL20 gene such as to cause suppression of the expression of the endogenous AGL20 gene and said first RNA nucleotide sequence and said second RNA nucleotide sequence form a double stranded RNA, wherein the double stranded RNA participate in RNA interference of expression of said endogenous AGL20 gene.

In one embodiment, a transgenic sugar beet cell is provided, particularly a transgenic sugar beet plant, wherein the heterologous DNA is obtainable from a 0.28 Kb cDNA fragment consisting of exons 3 to 7 of the AGL20 gene fragment according to the invention and as described herein before, in a PCR reaction using a forward primer with the nucleotide sequence 5′-CTATGGATCCGCATGCTG ATCTCCTGATC-3′ (SEQ ID NO: 8) and a reverse primer with the nucleotide sequence 5′- [symbol] [symbol] [symbol] AAGA AGTTAAAAAGTCTCGAAC-3′ (SEQ ID NO: 9).

In a specific embodiment the transgenic sugar beet cell, particularly the transgenic sugar beet plant, according to the invention comprises a heterologous gene construct, wherein the heterologous DNA is depicted by SEQ ID NO: 5.

In still another specific embodiment the transgenic sugar beet cell, particularly the transgenic sugar beet plant, according to the invention comprises a heterologous gene construct, wherein said gene construct includes an inverted repeat, which when transcribed, forms a double stranded RNA molecule in said plant cell comprising said first and second RNA strands, wherein said double stranded RNA molecule triggers AGL20 gene silencing.

In a specific embodiment of the invention, a transgenic sugar beet cell is provided, particularly a transgenic sugar beet plant, comprising a heterologous DNA inserted between a promoter and terminator, which heterologous DNA is obtainable by
a. amplifying a 0.28 Kb cDNA fragment consisting of exons 3 to 7 of the 0.6 Kb AGL20 gene fragment according to the invention and as described herein before, in a recombinant PCR reaction using a forward primer with the nucleotide sequence 5′-CTATGGATCCGCATGCTG ATCTCCTGATC-3′ (SEQ ID NO: 8) and a reverse primer with the nucleotide sequence 5′- [symbol] [symbol] [symbol] AAGA AGTTAAAAAGTCTCGAAC-3′ (SEQ ID NO. 9);
b. amplifying a 0.19 Kb fragment comprising the ST-LS1 intron and flanking splicing sites using forward primer 5′-ATCCAACCGCGGACCTGCACATCAACAA-3′ (SEQ ID NO: 7) and reverse primer 5′- [symbol] [symbol] [symbol] AGGTAAGTTTCTGCTTCTAC-3′ (SEQ ID NO: 12);
c. fusing the amplification products obtained in steps a) and b) to each other by means of a second round of PCR using primers of SEQ ID NO:8 and SEQ ID NO:7 and using a mix of both amplification products as template, yielding a fusion product of 0.47 Kb in length;
d. amplifying the 0.28 Kb BvAGL20 fragment a second time, using forward primer 5′-TAAATCCGCGGAAGAAGTTAAAAAGTCTCGAAC-3′ (SEQ ID NO: 10) and reverse primer 5′-CTATTTGTCGACGCATGCTGATCTCCTGATC-3′ (SEQ ID NO: 11) that differ from the primer used in step a) with respect to their linkers;
e. fusing both fragments at the Sac II restriction sites to create an inverted repeat for the BvAGL21 sequence separated by the intron from the potato ST-LS1 gene.

In another specific embodiment the transgenic sugar beet cell, particularly the transgenic sugar beet plant, according to the invention comprises a heterologous gene construct, wherein said gene construct comprises an expression cassette depicted by nucleotide sequence 233-2657 of SEQ ID NO: 2.

In another specific embodiment of the invention, a transgenic sugar beet cell, particularly a transgenic sugar beet plant, is provided comprising an expression cassette according to the invention and as described herein before.

In one embodiment, the invention relates to a method of producing a transgenic sugar beet plant according to the invention and as described herein before comprising:
a. transforming a sugar beet cell with an expression cassette according to the invention and as described herein before;
b. identifying a sugar beet cell containing the heterologous DNA,
c. regenerating a transgenic plant from said plant cell identified in step b)
d. identifying a sugar beet plant exhibiting a delay of the vernalization response or a complete suppression of the vernalization response resulting in a non bolting (NB) phenotype,
e. optionally confirming the presence of the heterologous DNA in the plant cell genome introduced in step a)

The invention also includes a method of suppressing the expression of an AGL20 gene in a sugar beet plant cell, comprising introducing into the plant cell a first RNA fragment that is sufficiently identical or complementary to a portion of the AGL20 gene, a second RNA fragment that is sufficiently complementary to the first RNA fragment, to result in the suppression of the expression of the endogenous AGL20 gene, wherein the first and second RNA fragments form a double stranded RNA molecule in the plant cell, wherein the double stranded RNA molecule suppresses by siRNA mediated silencing the expression of the AGL20 gene.

The invention also includes a method of suppressing the expression of an endogenous AGL20 gene in a sugar beet plant, comprising:
a) introducing into a sugar beet plant cell a first RNA strand;
b) growing said plant cell into a first plant;
c) introducing into a second sugar beet plant cell a second RNA strand, wherein said first RNA strand is sufficiently complimentary to at least a portion of a RNA strand of said endogenous AGL20 gene to hybridize or anneal to the RNA produced by the AGL20 gene such as to cause suppression of the expression of the endogenous AGL20 gene and said first RNA strand and said second RNA strand are capable of forming a double stranded RNA;
d) growing said second sugar beet plant cell into a second plant;
e) crossing said first plant with said second plant to produce seed; and
f) growing a plant from said seed, wherein said first and second RNA strands form double stranded RNA which participates in RNA interference of expression of said endogenous AGL20 gene.

In a specific aspect, the invention relates to a transgenic sugar beet cell, particularly a transgenic sugar beet plant, comprising in its genome
i. a first heterologous gene construct comprising the coding region of a heterologous or of an endogenous FLC gene, and
ii. a second heterologous gene construct capable of encoding a RNA composition, said construct comprising a heterologous DNA, which when transcribed, yields a first RNA nucleotide sequence and a second RNA nucleotide sequence, wherein said first RNA nucleotide sequence is sufficiently complimentary to at least a portion of a RNA strand of said endogenous AGL20 gene to hybridize or anneal to the RNA produced by the AGL20 gene and said first RNA nucleotide sequence and said second RNA nucleotide sequence form a double stranded RNA, wherein the double stranded RNA participate in RNA interference of expression of said endogenous AGL20 gene.

In one embodiment of the invention, a transgenic sugar beet cell, particularly a transgenic sugar beet plant, is provided, wherein said first heterologous gene construct comprises the FLC coding region consisting of the FLC cDNA of Accession No. AF537203.

In a specific embodiment of the invention said FLC gene is depicted by SEQ ID NO: 3.

In another embodiment of the invention, a transgenic sugar beet cell, particularly a transgenic sugar beet plant, is provided, wherein said FLC gene comprises the FLC coding region which has at least 99%, 98%, 97%, 96%, 95%, 94% or 93% sequence identity with the nucleotide sequence of the FLC cDNA of Accession No. AF537203.

In a specific embodiment of the invention said FLC gene has at least between 93% and 99% sequence identity with the nucleotide sequence depicted by SEQ ID NO: 3.

In another specific embodiment, said FLC gene comprises the FLC coding region of an endogenous FLC gene as depicted in SEQ ID NOs: 21, 23 and 25.

In still another specific embodiment, said FLC gene comprises an FLC coding region which has at least 99%, 98%, 97%, 96%, 95%, 94% or 93% sequence identity with the nucleotide sequence the FLC coding region of an endogenous FLC gene as depicted in SEQ ID NOs: 21, 23 and 25.

In one embodiment, the invention relates to a transgenic sugar beet cell, particularly a transgenic sugar beet plant, according to the invention and as described herein before, wherein said heterologous DNA comprised in the second gene construct is obtainable from a 0.28 Kb cDNA fragment consisting of exons 3 to 7 of the AGL20 gene fragment according to the invention and as described herein before, in a PCR reaction using a forward primer with the nucleotide sequence 5′-CTATGGATCCGCATGCTG ATCTCCTGATC-3′ (SEQ ID NO: 8) and a reverse primer with the nucleotide sequence 5′- [symbol] [symbol] [symbol] AAGA AGTTAAAAAGTCTCGAAC-3′ (SEQ ID NO: 9).

In one embodiment, the invention relates to a transgenic sugar beet cell, particularly a transgenic sugar beet according to the invention and as described herein before, wherein the heterologous DNA that transcribes said first RNA strand is depicted by SEQ ID NO: 5.

In one embodiment, the invention relates to a transgenic sugar beet cell or plant according to the invention and as described herein before, wherein said heterologous DNA comprised in the second gene construct includes an inverted repeat, which when transcribed, forms a double stranded RNA molecule in said plant cell comprising said first and second RNA strands, wherein said double stranded RNA molecule triggers AGL20 gene silencing.

In one embodiment, the inventions relates to a transgenic sugar beet cell or plant according to the invention and as described herein before, comprising in the second gene construct a heterologous DNA inserted between a promoter and terminator, which heterologous DNA is obtainable by
a. amplifying a 0.28 Kb cDNA fragment consisting of exons 3 to 7 of the 0.6 Kb AGL20 gene fragment according to the invention and as described herein before, in a recombinant PCR reaction using a forward primer with the nucleotide sequence 5′-CTATGGATCCGCATGCTG ATCTCCTGATC-3′ (SEQ ID NO: 8) and a reverse primer with the nucleotide sequence 5′- [symbol] [symbol] [symbol] AAGA AGTTAAAAAGTCTCGAAC-3′ (SEQ ID NO: 9);
b. amplifying a 0.19 Kb fragment comprising the ST-LS1 intron and flanking splicing sites using forward primer 5′-ATCCAACCGCGGACCTGCACATCAACAA-3′ (SEQ ID NO: 7) and reverse primer 5′- [symbol] [symbol] [symbol] [symbol] AGGTAAGTTTCTGCTTCTAC-3′ (SEQ ID NO: 12);
c. fusing the amplification products obtained in steps a) and b) to each other by means of a second round of PCR using primers of SEQ ID NO:8 and SEQ ID NO:7 and using a mix of both amplification products as template, yielding a fusion product of 0.47 Kb in length;
d. amplifying the 0.28 Kb BvAGL20 fragment a second time, using forward primer 5′-TAAATCCGCGGAAGAAGTTAAAAAGTCTCGAAC-3′ (SEQ ID NO: 10) and reverse primer 5′-CTATTTGTCGACGCATGCTGATCTCCTGATC-3′ (SEQ ID NO: 11) that differ from the primer used in step a) with respect to their linkers;
e. fusing both fragments at the Sac II restriction sites to create an inverted repeat for the BvAGL20 sequence separated by the intron from the potato ST-LS1 gene.

In one embodiment, the invention relates to transgenic sugar beet cell or plant according to the invention and as described herein before wherein said heterologous DNA comprised in the second gene construct is depicted by nucleotide sequence 233-2657 of SEQ ID NO: 2.

In one embodiment of the invention, a transgenic sugar beet cell, particularly a transgenic sugar beet plant, is provided, wherein said second heterologous gene construct is comprised in an expression cassette according to the invention and as described herein before.

In a specific aspect, the invention relates to a transgenic sugar beet plant as described herein before, wherein co-expression of the first and second heterologous gene construct leads to a synergistic delay of the vernalization response in said sugar beet plant.

In one embodiment, in invention thus relates to a transgenic sugar beet cell, particularly a transgenic sugar beet plant comprising the AGL20 and the FLC expression product in a synergistically effective amount.

In another specific aspect, the invention relates to a transgenic sugar beet plant as described herein before, wherein co-expression of the first and second heterologous gene construct leads to a complete suppression of the vernalization response in said sugar beet plant resulting in a non-bolting (NB) phenotype.

In still another specific aspect, the invention relates to a transgenic sugar beet plant as described herein before which plant is obtainable by a cross of two parent plants wherein the first heterologous gene construct is contributed by parent 1 and the second heterologous gene construct is contributed by parent 2, wherein at least one of the parent plants does not exhibit a non bolting (NB) phenotype.

In one embodiment, the invention relates to a method of producing a transgenic sugar beet plant according to the invention comprising:
a. transforming a sugar beet cell with an expression cassette comprising a heterologous FLC gene wherein said FLC gene is operably linked to regulatory sequences and/or an expression cassette according to the invention and as described herein before comprising a heterologous gene construct capable of suppressing expression of an endogenous AGL20 gene;
b. identifying a sugar beet cell containing the heterologous DNA,
c. optionally transforming the sugar beet cell identified in step b) with an expression cassette comprising a heterologous FLC gene wherein said FLC gene is operably linked to regulatory sequences or with an expression cassette according to the invention and as described herein before comprising a heterologous gene construct capable of suppressing expression of an endogenous AGL20 gene and identifying a sugar beet cell containing both the introduced heterologous DNAs;
d. regenerating a transgenic plant from said plant cell identified in step b)
e. identifying a sugar beet plant exhibiting a delay of the vernalization response or a complete suppression of the vernalization response resulting in a non bolting (NB) phenotype,
f. optionally confirming the presence of the heterologous DNAs in the plant cell genome introduced in step a) and, optionally, step c).

In a specific embodiment, the method of producing a transgenic sugar beet plant comprises crossing of two parent plants wherein the first heterologous gene construct is contributed by parent 1 represented by a sugar beet plant comprising a FLC gene according to the invention and as described herein before and the second heterologous gene construct is contributed by parent 2 represented by a sugar beet plant comprising a heterologous gene construct capable of suppressing expression of an endogenous AGL20 gene according to the invention and as described herein before; and to the plant resulting from said cross, particularly a plant that contains the gene construct contributed by both parent 1 and parent 2, more particularly a plant that contains the gene construct contributed by both parent 1 and parent 2 and exhibits the delayed vernalization response or non-bolting phenotype.

In a specific embodiment, the invention relates to a method of producing a transgenic sugar beet plant, wherein at least one of the parent plants does not exhibit a non bolting (NB) phenotype.

The present invention includes a root of the transgenic sugar beet plant according to the invention and as described herein before, wherein the root is derived from a plant derived from a transgenic sugar beet cell comprising
a) a heterologous gene construct, said construct comprising a heterologous DNA, which when transcribed in the sugar beet cell, yields a first RNA nucleotide sequence and a second RNA nucleotide sequence, wherein said first RNA nucleotide sequence is sufficiently complimentary to at least a portion of a RNA strand of said endogenous AGL20 gene to hybridize or anneal to the RNA produced by the AGL20 gene such as to cause suppression of the expression of the endogenous AGL20 gene and said first RNA nucleotide sequence and said second RNA nucleotide sequence form a double stranded RNA, wherein the double stranded RNA participate in RNA interference of expression of said endogenous AGL20 gene
b) a heterologous gene construct, said construct comprising a heterologous or an endogenous FLC gene
c) a combination of a) and b).

The present invention includes a plant derived from a transgenic sugar beet cell according to the invention and as described herein before comprising a heterologous gene construct, said construct comprising
a) a heterologous DNA, which when transcribed in the sugar beet cell, yields a first RNA nucleotide sequence and a second RNA nucleotide sequence, wherein said first RNA nucleotide sequence is sufficiently complimentary to at least a portion of a RNA strand of said endogenous AGL20 gene to hybridize or anneal to the RNA produced by the AGL20 gene such as to cause suppression of the expression of the endogenous AGL20 gene and said first RNA nucleotide sequence and said second RNA nucleotide sequence form a double stranded RNA, wherein the double stranded RNA participate in RNA interference of expression of said endogenous AGL20 gene.
b) a heterologous gene construct, said construct comprising a heterologous or an endogenous FLC gene
c) a combination of a) and b).

The present invention also includes a progeny plant derived from a transgenic sugar beet plant according to the invention and as described herein before comprising a heterologous gene construct, said construct
a) heterologous DNA, which when transcribed in the sugar beet cell, yields a first RNA nucleotide sequence and a second RNA nucleotide sequence, wherein said first RNA nucleotide sequence is sufficiently complimentary to at least a portion of a RNA strand of said endogenous AGL20 gene to hybridize or anneal to the RNA produced by the AGL20 gene such as to cause suppression of the expression of the endogenous AGL20 gene and said first RNA nucleotide sequence and said second RNA nucleotide sequence form a double stranded RNA, wherein the double stranded RNA participate in RNA interference of expression of said endogenous AGL20 gene.
b) a heterologous gene construct, said construct comprising a heterologous or an endogenous FLC gene
c) a combination of a) and b).

The present invention includes sugar derived from the transgenic sugar beet root comprising heterologous gene construct, said construct comprising
a) heterologous DNA, which when transcribed in the sugar beet cell, yields a first RNA nucleotide sequence and a second RNA nucleotide sequence, wherein said first RNA nucleotide sequence is sufficiently complimentary to at least a portion of a RNA strand of said endogenous AGL20 gene to hybridize or anneal to the RNA produced by the AGL20 gene such as to cause suppression of the expression of the endogenous AGL20 gene and said first RNA nucleotide sequence and said second RNA nucleotide sequence form a double stranded RNA, wherein the double stranded RNA participate in RNA interference of expression of said endogenous AGL20 gene.
b) a heterologous gene construct, said construct comprising a heterologous or an endogenous FLC gene
c) a combination of a) and b).

The present invention includes biofuels such as ethanol, butanol, methanol, biogas and diesel derived from the transgenic sugar beet root comprising heterologous gene construct, said construct comprising
a) heterologous DNA, which when transcribed in the sugar beet cell, yields a first RNA nucleotide sequence and a second RNA nucleotide sequence, wherein said first RNA nucleotide sequence is sufficiently complimentary to at least a portion of a RNA strand of said endogenous AGL20 gene to hybridize or anneal to the RNA produced by the AGL20 gene such as to cause suppression of the expression of the endogenous AGL20 gene and said first RNA nucleotide sequence and said second RNA nucleotide sequence form a double stranded RNA, wherein the double stranded RNA participate in RNA interference of expression of said endogenous AGL20 gene.
b) a heterologous gene construct, said construct comprising a heterologous or an endogenous FLC gene
c) a combination of a) and b).

The present invention includes other industrial applications such as plastics derived from the transgenic sugar beet root comprising heterologous gene construct, said construct comprising
a) heterologous DNA, which when transcribed in the sugar beet cell, yields a first RNA nucleotide sequence and a second RNA nucleotide sequence, wherein said first RNA nucleotide sequence is sufficiently complimentary to at least a portion of a RNA strand of said endogenous AGL20 gene to hybridize or anneal to the RNA produced by the AGL20 gene such as to cause suppression of the expression of the endogenous AGL20 gene and said first RNA nucleotide sequence and said second RNA nucleotide sequence form a double stranded RNA, wherein the double stranded RNA participate in RNA interference of expression of said endogenous AGL20 gene.
b) a heterologous gene construct, said construct comprising a heterologous or an endogenous FLC gene
c) a combination of a) and b).

The present invention also includes a method of producing sugar, ethanol, biogas and/or diesel fuel comprising processing a sugar beet plant according to any of the preceding claims and deriving sugar from the sugar beet plant comprising a heterologous gene construct, said construct comprising
a) heterologous DNA, which when transcribed in the sugar beet cell, yields a first RNA nucleotide sequence and a second RNA nucleotide sequence, wherein said first RNA nucleotide sequence is sufficiently complimentary to at least a portion of a RNA strand of said endogenous AGL20 gene to hybridize or anneal to the RNA produced by the AGL20 gene such as to cause suppression of the expression of the endogenous AGL20 gene and said first RNA nucleotide sequence and said second RNA nucleotide sequence form a double stranded RNA, wherein the double stranded RNA participate in RNA interference of expression of said endogenous AGL20 gene.
b) a heterologous gene construct, said construct comprising a heterologous or an endogenous FLC gene
c) a combination of a) and b).

The present invention is further directed to novel compositions and methods relating to RNA interference (RNAi). The compositions include dsRNA containing RNA strands that are sufficiently complementary or identical to a target mRNA, such as AGL20 mRNA. As currently understood, the dsRNA are processed by Dicer by cutting the dsRNA into short interfering RNA (siRNA). According to one embodiment of the present invention, novel siRNA compositions are incorporated into the RISC complex for RNA, interference of a target gene mRNA, such as the sugar beet AGL20 gene mRNA. Interfering with sugar beet AGL20 gene mRNA expression results in suppression or delay of the sugar beet vernalization response. A delay in the vernalization response results in the sugar beet plant continuing its vegetative growth and to develop a normal taproot.

In one embodiment of the invention, the siRNA includes a first RNA strand that is between 21 and 23 nucleotides in length and a second RNA strand that hybridizes to the first sequence under biological conditions, such as those conditions found in the cell, particularly in the cytoplasm and/or the nucleus of the cell.

In yet another embodiment of the invention, the siRNA includes a first RNA strand that is between 19 and 30 nucleotides in length and a second RNA strand that hybridizes to the first sequence under biological conditions, such as those conditions found cell, particularly in the cytoplasm and/or the nucleus of the cell.

The invention includes siRNAs of any length, provided that the novel siRNA play a rote in triggering RNA interference of a target gene mRNA, such as the sugar beet AGL20 gene mRNA.

In another embodiment of the invention, the siRNA first or second strands are sufficiently complementary or identical to a nucleotide sequence of RNA produced by the AGL20 gene to trigger RNA silencing. The term “sufficient complementary” means that the first or second strand sequences of the siRNA are capable of hybridizing or annealing sufficiently to the RNA produced by the target gene (mRNA) under conditions found in the cytoplasm, such that RNAi is triggered which leads to a suppression of the expression of the target gene. This suppression of the AGL20 gene causes the sugar beet plant to develop a non-bolting phenotype which means that the sugar beet plant does no longer respond to a typical vernalization period of 18 weeks by bolting and subsequent flowering, but to the contrary continue vegetative growth (non-bolting) and develop a normal taproot.

Plants expressing said non-bolting phenotype can be easily identified and selected by applying a phenotypic analysis experiment employing standardized growth conditions.

In one embodiment, a siRNA molecule of the invention includes a nucleic acid strand that is sufficiently complementary or identical to at least a portion of the AGL20 gene. It is known that if the siRNA strand is identical, the target mRNA is cut into useless RNA fragments. However, if the pairing is less than identical, the RISC complex binds to the mRNA and is capable of blocking ribosome movement along the native mRNA, but is not capable of cutting the mRNA into small fragments. Nevertheless, in either case, expression of the gene from which the mRNA is transcribed, is silenced—no AGL20 protein is formed. The present invention, therefore, further includes one strand of the siRNA that is sufficiently complementary or identical to a corresponding sequence of the mRNA transcribed from the gene whose expression is altered. For example, the strand of the siRNA that binds to the mRNA is at least 50% identical to the corresponding mRNA sequence of the target gene, more desirably at least 70% identical, yet more desirable is at least 90% identity and even more desirable is at least 95% identical.

It is to be understood that the percentage of identity between the one strand of the siRNA that is sufficiently complementary or identical to a corresponding sequence of the mRNA transcribed from the gene whose expression is altered and the mRNA produced by the target gene, which is in the range of between at least 70% identity and at least 95% identity, can be any numerical value within this range.

It is known that RNA sequences with insertions, deletions, and single point mutations relative to the target sequence are also effective for target gene expression suppression. Sequence identity between the siRNA molecule and the target gene transcription product (for example, the target gene mRNA) may be optimized by alignment algorithms known in the art and calculating the percent similarity between the nucleotide sequences. Alternatively, the siRNA molecule of the present invention may be identified not by its sequence similarity to the target molecule, but by its capability to hybridize to and silence expression of the target sequence.

According to another embodiment of the present invention, novel siRNA compositions can be used by RNA dependent RNA polymerase (RdRp) to make a new dsRNA, which can then be processed to form more siRNA.

Yet another embodiment of the invention occurs when the single stranded siRNA compositions of the invention not associated with RISC bind to their corresponding mRNA, for example AGL20 transcribed mRNA, wherein RNA dependent RNA polymerase serves as a primer to produce dsRNA.

In yet another embodiment of the invention, a method of gene silencing includes separately introducing into a plant cell a sense RNA fragment of a target gene, such as AGL20, and an antisense RNA fragment of the same gene, wherein the sense RNA fragment and the antisense RNA are capable of forming a double-stranded RNA molecule, wherein the expression of the target gene in the cell is altered. In a preferred embodiment, the RNA fragments are comprised in two different RNA molecules. In another preferred embodiment, the RNA fragments are mixed before being introduced into the cell under conditions allowing them to form a double-stranded RNA molecule. In another preferred embodiment, the RNA fragments are introduced into said cell sequentially. In yet another embodiment, the RNA fragments are comprised in one RNA molecule. In such case, the RNA molecule is preferably capable of folding such that said RNA fragments comprised therein form a double stranded RNA molecule. Various methods of using sense and antisense RNA fragments to silence a target gene are described in WO99/61631.

The present invention further provides for a method of introducing into a plant cell a dsRNA molecule comprises of sense and antisense fragments of a target gene mRNA.

It is understood, however, that the underlying mechanics of RNAi silencing may not be entirely understood, and thus the present invention is not be bound to any particular RNAi silencing theory. Thus, in the context of the present invention, the term “siRNA mediated silencing” is 5 not restricted to a particular RNA interference cellular mechanism.

### EXAMPLES

### Example 1

### Assembly of the Binary Transformation Vector for the Constitutive Expression of FLC in Transgenic Sugar Beet

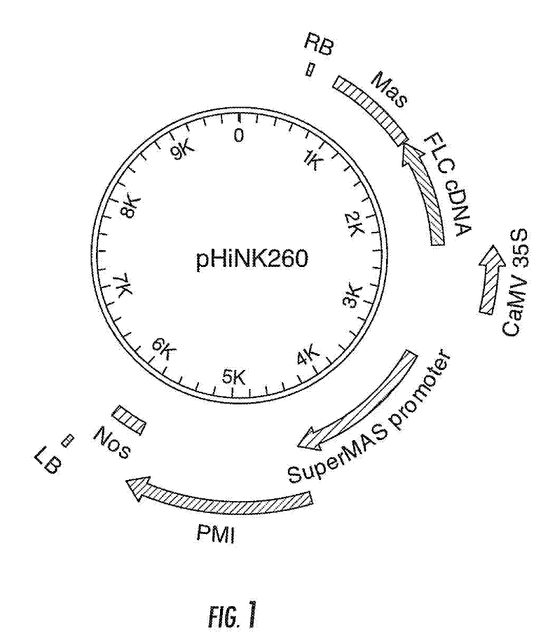

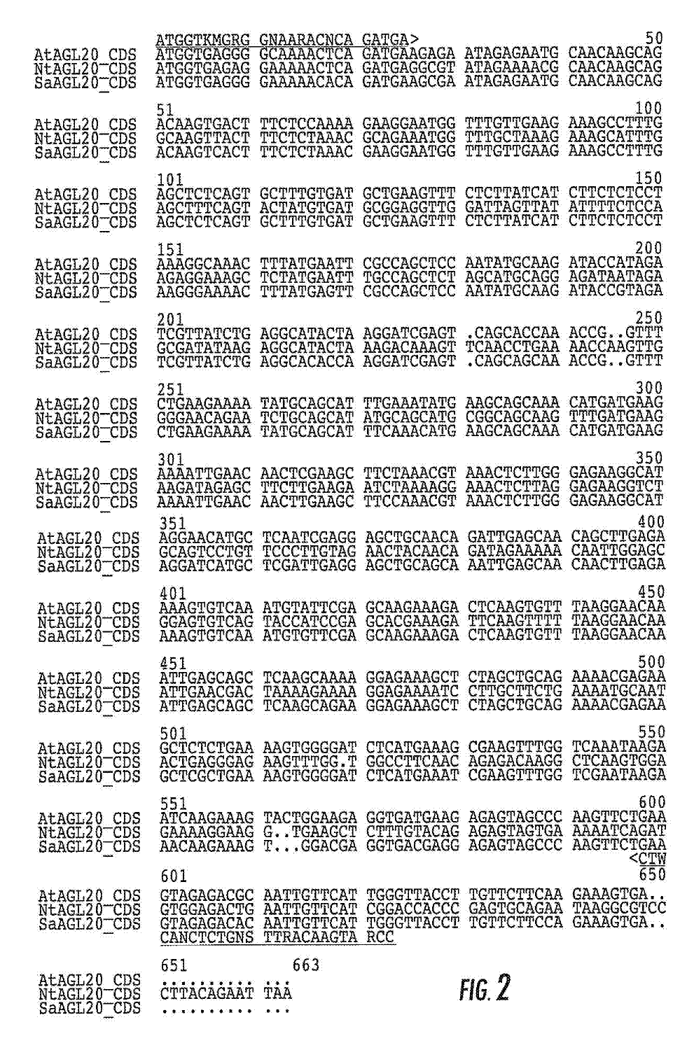

The FLC gene cassette is under the control of the constitutive CaMV 35S promoter. The FLC coding region consists of the FLC cDNA from Arabidopsis thaliana (Accession No. AF537203, SEQ ID NO: 3) followed by the mannopine synthase (mas) terminator from Agrobacterium tumefaciens. The gene cassette was introduced as a 2.4 Kb Aso I-Pac I fragment on the T-DNA of the proprietary binary transformation vector pVictorHiNK carrying the SuperMAS::PMI::NOS selectable marker gene for mannose selection in sugar beet (Joersbo et al, 1998), yielding binary vector pHiNK260 (FIG. 1). The complete nucleotide sequence of pHiNK260 is disclosed in SEQ. 1. Upon completion, binary vector pHiNK260 was transformed into Agrobacterium tumefaciens strain EHA101 (Hood et al., 1986) by means of a heatshock as described in Holsters et al., 1978.

### Example 2

### Amplification and Cloning of Homologues from Sugar Beet Beta vulgaris

2.1 Amplification and Cloning of the Putative FLC Homologue from Sugar Beet.

In order to amplify and clone the FLC homologue from sugar beet, degenerate primers were designed against the conserved MADS_MEF2-like domain that is present at the NH2 terminus of all Type 2 members of the MADS box family of transcription factors to which FLC belongs (Pa\check{r}enicová et al, 2003). Degenerate primer HiNK5277 (5′-CGNCGNAAYGGNCTNCTNAARAARGC-3′, SEQ ID NO: 30) targets the conserved amino acid sequence motif “RRNGLLKKA”; primer HiNK5279 (5′-GCNTAYGARCTNTCNGTNCTNTGYGAYGCNGA-3′, SEQ ID NO:31) hybridizes immediately downstream of HiNK5277 and targets amino acid sequence motif ‘AYELSVLCDAE’.

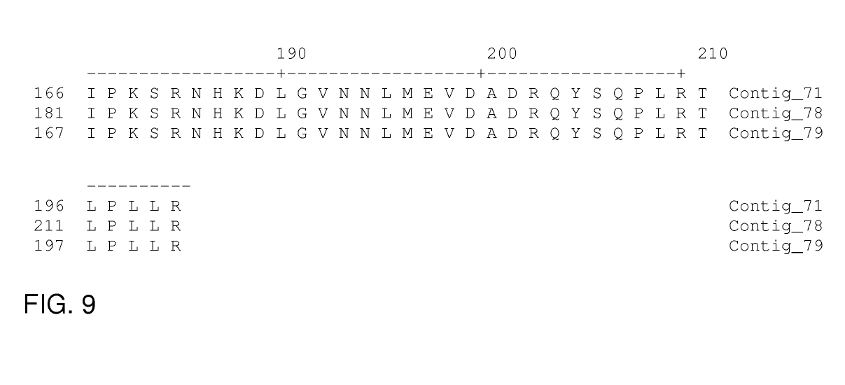

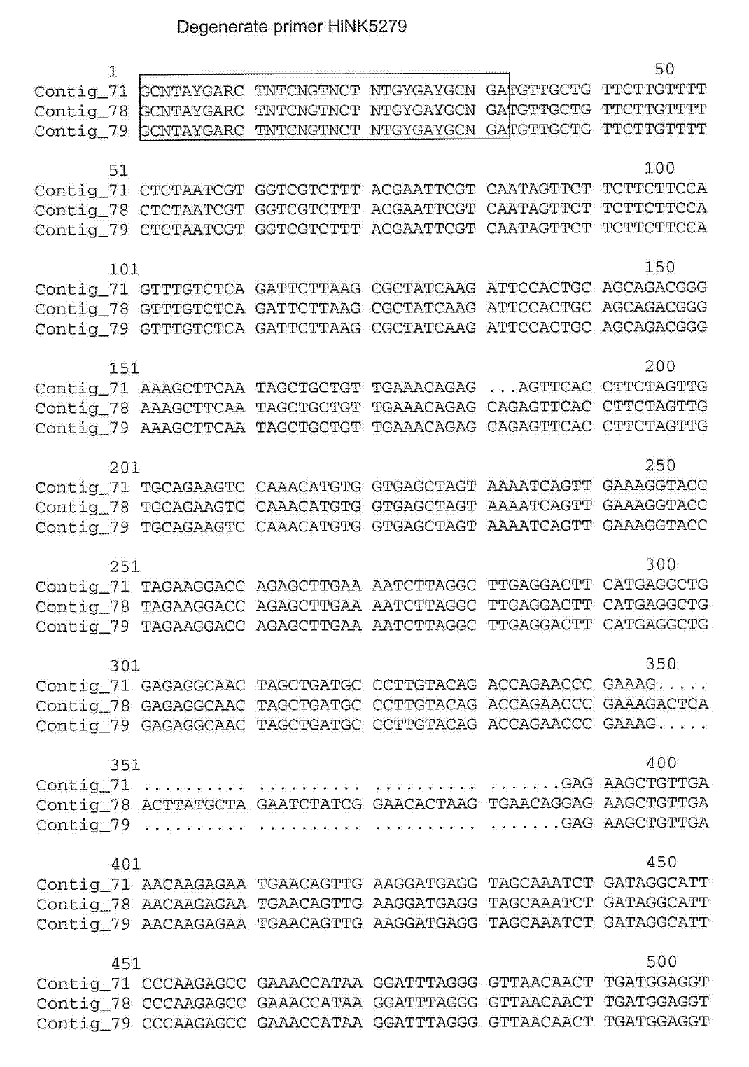

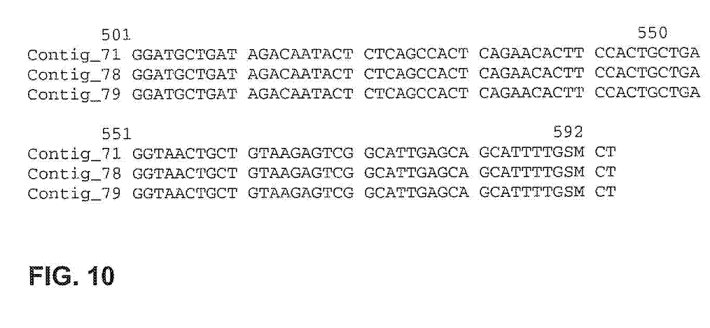

Total RNA was extracted from sugar beet leaves and apices using the RNeasy Plant Mini kit from Qiagen and converted into cDNA using the FirstChoice RLM-RACE kit from Ambion, Inc. Experimental conditions were essentially as described by the 3′ RLM-RACE protocol supplied with the kit using the 3′ RACE adapter as primer in the reverse transcriptase reaction. The putative FLC homologue was subsequently amplified starting from the cDNA reaction as initial template in two successive rounds of PCR using the 3′ RACE Outer Primer in combination with degenerate primer HiNK5277 followed by the combination of the 3′ RACE Inner Primer with degenerate primer HiNK5279 in typical PCR reactions. The thus obtained amplification fragment measured approximately 0.6 Kb in size as expected according to the sequence of the FLC homologues from Brassica species. However, the obtained DNA band is expected to contain multiple sequences due to the degenerate nature of primers HiNK5277 and HiNK5279 that in principle will allow for the amplification of multiple members of the MAIDS box family of transcription factors. The PCR products were excised, purified, cloned and subsequently submitted for sequence analysis. Amongst the various sequences obtained that, as expected all share part of the MADS box motif, three highly homologous sequences were identified as putative homologues of FLC, referred to as contig_{—}71, _{—}78 and _{—}79. These three cDNA fragments differ from each other by small in-frame deletions which suggest that they represent alternative splicing variants of one and the same gene. At the 5′ end all three cDNA fragments show extensive sequence homology to a public sugar beet EST with accession number BQ595637. Combining the EST sequence with the three cDNA fragments allowed for the reconstitution of the full-length cDNAs transcribed from the putative FLC homologue (SEQ ID NO 21, 23 and 25) and the corresponding translation products (SEQ ID NO 22, 24 and 26). The alignment, of three putative FLC proteins is shown in FIG. 9.

2.2 Amplification and Cloning of the AGL20 Homologue from Sugar Beet.

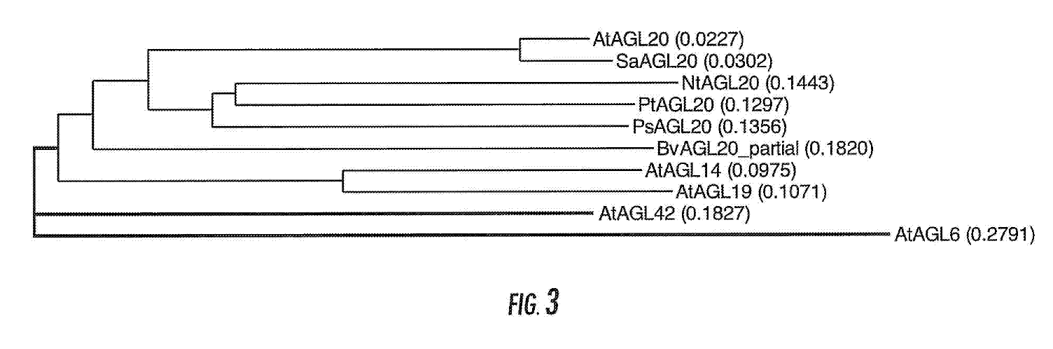

In order to amplify and clone the AGL20 homologue from sugar beet, degenerate primers were designed against the conserved nucleotide sequences when aligning the AGL20 cDNA from Arabidopsis to the AGL20 homologues from mustard and tobacco (FIG. 2). Since AGL20 belongs to the large family of MADS box transcription factors, the primers were designed to regions conserved between the various AGL20 homologues, but distinct to most of the other MADS box family members. Primer HiNK624 (5′-ATGGTKMGRGGNAARACNCAGATGA-3′, SEQ ID NO: 13), shares sequence homology to the extreme NH2-terminus starting at the ATG codon and spanning codons 1 to 9; primer HiNK619 (5″-CCRATGAACARTTSNGTCTCNACWTC-3′, SEQ ID NO: 14), is complementary to the COOH-terminus, hybridizing just upstream of the stop codon at exon 81 spanning codons 198 to 206 according to the AGL20 sequence from Arabidopsis.

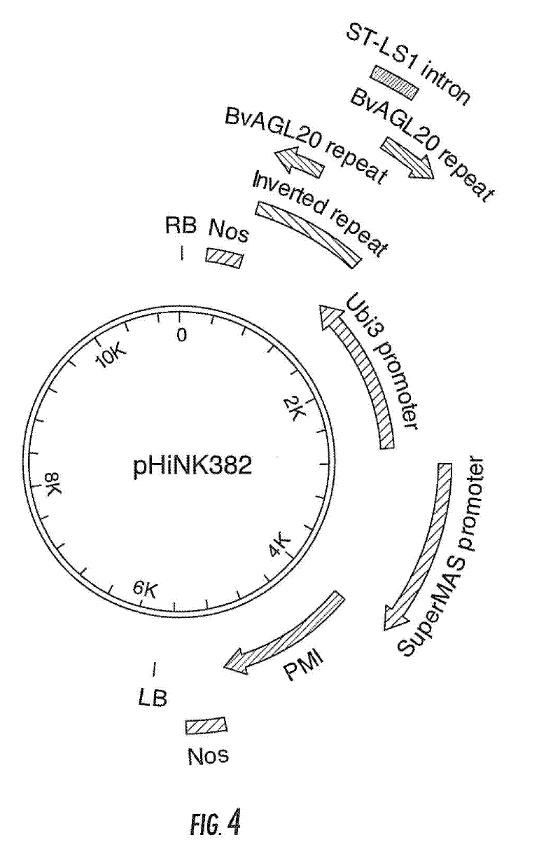

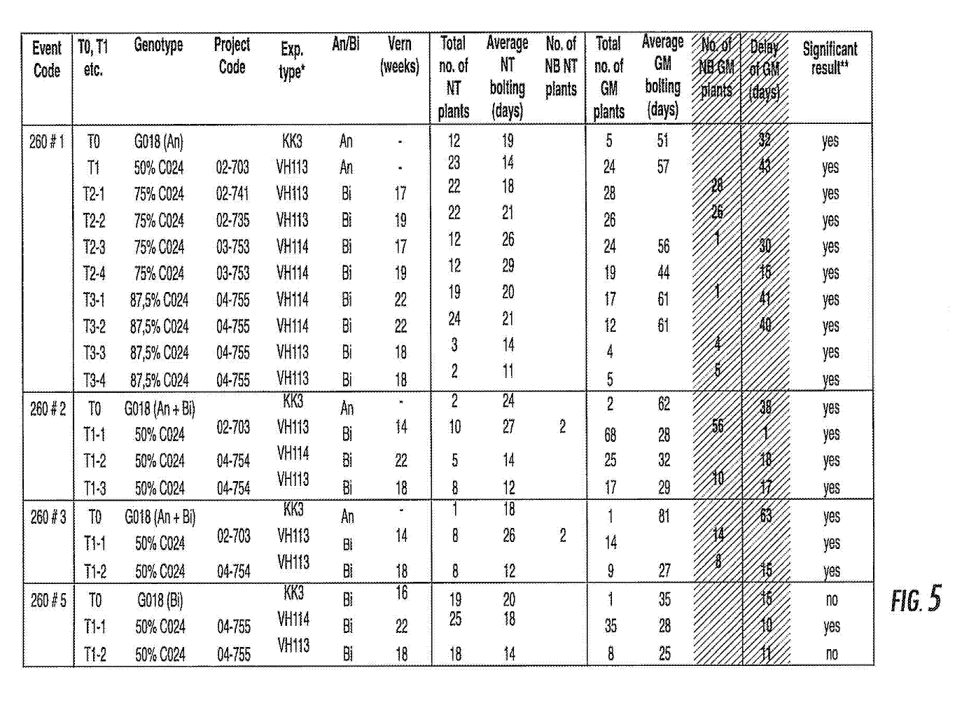

Total RNA was extracted from sugar beet leaves using the RNeasy Plant Mini kit from Qiagen and converted into cDNA using the Superscript™ II RNase H⁻ reverse transcriptase from Life Technologies. Experimental conditions were essentially as described by the suppliers, but using HiNK619 as primer in the reverse transcriptase reaction. The putative AGL20 homologue was amplified starting from the cDNA reaction as template and using primers HiNK619 and HiNK624 in a typical PCR reaction. The thus obtained amplification fragment measured approximately 0.6 Kb in size as was expected according to the sequence of the AGL20 homologues from heterologous species. Upon cloning and sequence analysis, the nucleotide sequence of the sugar beet homologue as listed in FIG. 3 was shown to share strong homology to the AGL20 gene from Arabidopsis (FIG. 4) and is referred to as BvAGL20 hereinafter (SEQ ID NO: 6). Albeit that the BvAGL20 fragment did not share as strong homology as observed for the AGL20 homologues from other species, including pine and pea, the homology to AGL20 was stronger than to the any of the other members of the MADS box transcription factors from Arabidopsis. A partial genomic sequence including introns 2 to 6 was obtained by designing primers to exons 2 and 7 HiNK725 (5′-ACTAAGACAATTATCGGTACCAAAGC-3′, SEQ ID NO: 15) and HiNK729 (5′-AAGGTAGCAGATCTGGTGAAGAATTGAG-3′, SEQ ID NO: 16), respectively that were used to amplify and sequence the genomic fragment obtained using sugar beet DNA as template in a typical PCR reaction. The partial genomic sequence of the sugar beet homologue (SEQ ID NO: 4) showed strong conservation with respect to the position of the intervening sequences when compared to the Arabidopsis sequence, regardless of the fact that the introns in sugar beet are substantially longer than in Arabidopsis.

### Example 3

### Assembly of the Binary Transformation Vector for RNAi Induced Suppression of the AGL20 Gene in Transgenic Sugar Beet

By means of a strategy known as ‘recombinant-PCR’ (Higuchi, 1990), a 0.28 Kb cDNA fragment consisting of exons 3 to 7 of the AGL20 homologue from sugar beet (SEQ ID NO: 5) was fused to the second intron from the potato ST-LS1 gene (Eckes et al., 1986; Vancanneyt et al., 1990). Care was taken not to include the MADS domain to prevent suppression of other MADS box transcription factors due to the strong sequence conservation of the MADS domain amongst the family of MADS box transcription factors. The BvAGL20 fragment was amplified using primers HiNK792 (5′-CTATGGATCCGCATGCTG ATCTCCTGATC-3′, SEQ ID NO: 8) and 793 (5′- [symbol] [symbol] [symbol] AAGA AGTTAAAAAGTCTCGAAC-3′, SEQ ID NO, 9), the first carrying a short linker to add a BamH I restriction site, the latter carrying a tail of 17 nucleotides complementary to the 5′ end of the ST-LS1 intron (linkers and tails are underlined hereinafter). The 0.19 Kb fragment comprising the ST-LS1 intron and flanking splicing sites was amplified using primers HiNK529 (5′-ATCCAACCGCGGACCTGCACATCAACAA-3′, SEQ ID NO; 7) and 796 (5′- [symbol] [symbol] [symbol] AGGTAAGTTTCTGCTTCTAC-3′, SEQ ID NO: 12), HiNK529 carrying a linker including the recognition sequence of Sac II and HiNK796 carrying a tail of 22 nucleotides identical to the 5′ extremity of the 0.28 Kb BvAGL20 fragment. As a consequence of the added tails, primers HiNK793 and 796 as well as their cognate amplification products are complementary to each other over a length of 39 nucleotides. By virtue of this overlap both amplification products were fused to each other by means of a second round of PCR using primers HiNK792 and 529 and using a mix of both amplification products as template, yielding a fusion product of 0.47 Kb in length. The 0.28 Kb BvAGL20 fragment was amplified a second time, now using primers HiNK794 (5′-TAAATCCGCGGAAGAAGTTAAAAAGTCTCGAAC-3′, SEQ ID NO: 10) and 795 (5′-CTATTTGTCGACGCATGCTGATCTCCTGATC-3′, SEQ ID NO: 11) that differ from HiNK793 respectively HiNK792 with respect to their linkers only; HiNK794 and 795 carry 5′ linkers to add a Sac II and a Sal I recognition sequence respectively. Both fragments were fused at the Sac II restriction sites to create an inverted repeat for the BvAGL20 sequence separated by the intron from the potato ST-LS1 gene. The intron was included as spacer fragment to stabilize the inverted repeat, but also to improve the efficiency of the RNAi phenomenon in the future transgenic events (Wang and Waterhouse, 2001; Smith et al., 2000). The thus obtained inverted repeat of approximately 0.75 Kb was subsequently introduced between the Ubi3-promoter from Arabidopsis (Norris et al., 1993) and the nos terminator from Agrobacterium tumefaciens as a BamH I-Sal I fragment. Subsequently, the gene cassette was transferred as a 2.5 Kb Asc I-Pac I fragment onto the T-DNA of the proprietary binary transformation vector pVictorHiNK, yielding pHiNK382, next to the SuperMAS::PMI::NOS selectable marker gene for mannose selection (FIG. 4). The complete nucleotide sequence of pHiNK382 is disclosed in SEQ. ID NO: 2.

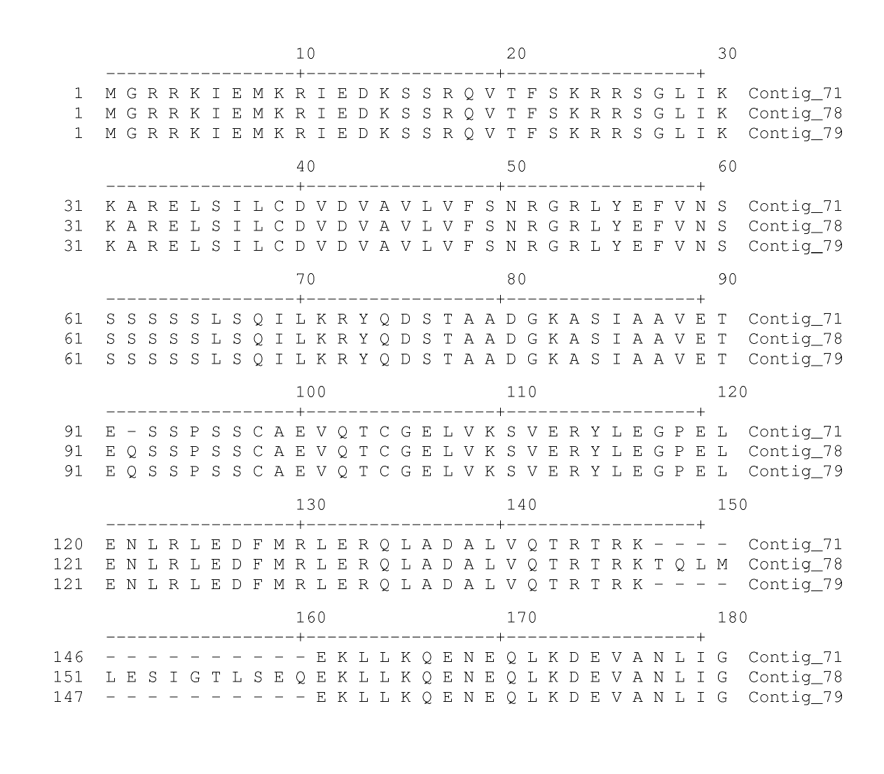

Another embodiment of the invention includes two more binary vectors: pHiNK440 and pHiNK441, that were assembled for the transgenic expression of the BvAGL20 cDNA fragment in sugar beet. Contrary to pHiNK382 that carries an inverted repeat for the BvAGL20 cDNA fragment resulting in the instant formations of a dsRNA or hairpin upon expression of the gene cassette, pHiNK440 and 441 only express the sense or antisense orientation, respectively, of the BvAGL20 cDNA fragment. The dsRNA for BvAGL20 is therefore obtained after crossing events for either vector to each other, resulting in the simultaneous accumulation of the sense and antisense orientation of the cDNA fragment, and in the subsequent formation of a dsRNA. The gene cassettes for the sense (pHiNK40) and antisense (pHiNK441) expression were obtained by amplifying the same 0.28 Kb BvAGL20 fragment (SEQ ID NO: 5) using primers HiNK2617 (5′-TAAATGGATCC AAGAAGTTAAAAAGTCTCGAAC-3′, SEQ ID NO: 17) and HiNK795, respectively primers HiNK2618 (5′-GAAGCAGAAACTTACCTGTCGACAAGAAGTTAAAAAGTCT CGAAC-3′, SEQ ID NO: 18) and HiNK792, and the subsequent cloning of the amplification products as BamHI-SalI fragment between the Ubi3 promoter and the nos terminator. As in the case of pHiNK382, the gene cassettes were subsequently transferred as Ase I-Pac I fragments onto the T-DNA of the proprietary binary transformation vector pVictorHiNK that already carried the SuperMAS::PMI::NOS selectable marker for mannose selection, yielding pHiNK440 and 441 (FIGS. 7 and 8). The complete nucleotide sequences of binary vectors pHiNK440 and 441 are disclosed in SEQ. 19 and SEQ. 20 respectively. Upon completion all binary vectors were transformed into Agrobacterium tumefaciens strain EHA101 by means of the heatshock protocol described in Holsters et al., 1978.

Therefore, the present invention further includes providing an expression cassette comprising a BvAGL20 cDNA fragment oriented in the sense direction and a second expression cassette comprising the BvAGL20 cDNA fragment oriented in the antisense direction. In one embodiment, the expression cassette including a BvAGL20 cDNA fragment oriented in the sense direction is pHiNK440, wherein an expression cassette including the BvAGL20 cDNA fragment oriented in the antisense direction is pHiNK441.

The present invention further includes a method for the conditional RNAi suppression of endogenous sugar beet expression of AGL20, wherein the method includes: (a) transforming a male or a female sugar beet parental inbred line with a BvAGL20 cDNA fragment oriented in the sense direction and transforming a female or a male parental inbred line with the BvAGL20 cDNA fragment oriented in the antisense direction; (b) crossing the female and male parental lines of (a) to produce a hybrid sugar beet plant, wherein the sense and antisense cDNA fragments form dsRNA in the hybrid sugar beet plant resulting in bolting control of said hybrid plant.

The present invention recognizes that parental lines comprising only a sense or antisense BvAGL20 cDNA fragment will not undergo RNAi of AGL20 expression and therefore will develop to produce flowers and seed for generation of progeny. Only when the sense and antisense fragments are combined in the hybrid plant do the RNAi mechanisms cause suppression of bolting, thereby allowing sugar beet to be sown in autumn in northern latitudes without the risk of bolting and flowering in the following season. This shifts the sugar beet from a traditional spring crop into a winter crop, which permits growers to drill their crop in autumn and to harvest the next summer. It has been shown that winter cultivars typically produce higher yields compared to spring cultivars.

### Example 4

### Transformation

Intact sugar beet seeds were surface sterilized, germinated and pretreated in vitro. Explants were then transformed via Agrobacterium tumefaciens mediated gene transfer, using the multiple shoot protocol disclosed in WO 02/14523 A2.

### 4.1 Seed Sterilization and Germination

Seeds of sugarbeet (Beta vulgaris L.) are surface sterilized and plated onto seed germination medium (GM) under aseptic culture. The GM comprised may contain Murashige and Skoog (MS) salts with about 30 g/L sucrose, myo-inositol (100 mg/L), pantothenic acid (1 mg/L) and appropriate gelling agent were also included in the GM, as were plant growth regulators with cytokinin-like function. Cytokinin levels are generally within a typical range of 0.5 mg/L to 5 mg/L, and usually between 1.0 and 2.0 mg/L. The auxin inhibitor TIBA is also added.

4.2 Excision and Initiation of Shoot Meristematic Cultures aShoot tips of 10-20 day old seedlings are excised and plated onto shoot multiplication medium (SMM). In this case, the SMM comprised Murashige and Skoog salts with 30 g/L sucrose and appropriate gelling agent. In addition, the SMM contained at least one cytokinin growth regulator such as BA, kinetin, 2-ip or zeatin, generally within a concentration range of about 1 to 10 mg/L and usually within a concentration range of 1-5 mg/L. The shoot tips consisted of both apical and axillary shoot meristematic regions, leaf primordia, 5 mm of hypocotyl and the cotyledonary leaves which are cut off to reduce further elongation. Every 7-10 days following plating, target explants were subcultured to fresh SMM after removing any new elongated leaf material. Multiple shoot target explants are; typically cultured under low light intensity (10-30 μEinsteins) for 16 hour day-lengths at ca 21-22° C. After 4 to 7 weeks the multiple shoot cultures resemble compact rosettes and are ready for transformation.

### 4.3 Inoculation and Incubation of Multiple Shoot Culture

Agrobacterium tumefaciens mediated transformation is utilized for the transformation of the multiple shoot culture. The A. tumefaciens strain EHA101 containing the binary vectors according to the invention (e.g. pHiNK260; pHiNK382; pHiNK440 and 441) is grown on solid culture medium consisting of 1 g/L yeast extract, 5 g/L peptone and appropriate gelling agent for 2-3 days at 28° C. One day prior to transformation the multiple shoot culture is prepared for inoculation by removing any remaining elongated leaf material.

To begin inoculation, single colonies of A. tumefaciens are collected together on the original YEB culture plate using a sterile loop. For the actual inoculation of each target explant, a sterile scalpel blade is dipped into the collected A. tumefaciens colonies and used to make cuts in the apical and axillary meristem regions of each target. Immediately following this inoculation step, about 7 μl of MSMG Induction Medium (MS salts, 2 g/L Glucose, MES, and 200 μM acetosyringone) is applied to the wounded surface of each target in some experiments. An effort is made to cut through the center of as many meristematic zones as possible in order to direct gene delivery to shoot meristem producing cells. Ten to twenty target cultures are typically treated in sequence and then allowed to air dry under sterile conditions in a laminar flow hood for 10 minutes. Following the air drying treatment, treated target explants are moved to MSCC co-cultivation medium (MS salts, 85 vitamins, 2 mg/L BA, 30 g/L sucrose, 200 μM acetosyringone with appropriate gelling agent). The treated explants are then incubated on MSCC medium for 2-4 days at 21-22° C. with continuous dark culture.

### 4.4 Target Culture and Selection

Following inoculation and co-cultivation, the multiple shoot explants are transferred to fresh SMM medium with 2 mg/L BA and appropriate antibiotics and gelling agent for a minimum of four days before applying mannose selection pressure. Transformed tissues are selected on gradually increasing amounts of mannose (2.5 g/L-15 g/L) and decreasing amounts of sucrose (20 g/L-3 g/L) following transformation. Mannose selection levels are increased in a stepwise manner, from 2.5 g/L mannose+20 g/L sucrose to 4 g/L mannose+20 g/L sucrose, followed by 5 g/L mannose+20 g/L sucrose, followed by 6 g/L mannose+18 g/L sucrose and 8 g/L mannose+15 g/L sucrose. The multiple shoot cultures continue to grow in size and are carefully divided at each sub-culturing to promote adequate selection pressure. During this period, the BA level is reduced to 0.25 mg/L and then eliminated to promote shoot elongation. Areas of surviving transformed tissue are continually removed from dying untransformed sections of the original target explant and surviving sections are again carefully divided to promote stringent selection. Selection and shoot regeneration typically progress over a time period of from 10 to about 30 weeks. As young shoots emerge they are separated and isolated under selection for the most efficient selection of transformed shoots.

### 4.5 Elongation of Transformed Shoots

Once the young shoots reach approximately 0.5-1.5 cm, they are transferred to containers with shoot elongation medium (elongation of developing shoots is enhanced by reduction of cytokinin levels) with mannose selection as described above. The shoot elongation medium containing MS salts, appropriate gelling agent and low levels of cytokinin are incorporated in the elongation medium, within a typical range of 0.1 to 1.0 mg/l. The optimal cytokinin application for sugar beet is 0.2 mg/L kinetin.

### 4.6 Regeneration of Transformed Plants

Selection of transgenic sugar beet shoots was performed on a standard regeneration medium supplemented with mannose-6-phosphate as selective agent (WO 94/20627).

Transformed shoots are cloned on MS-based cloning medium plus mannose at 5-15 g/L. Multiple shoots from one original transgenic shoot are sometimes desirable, and for this reason a combination of cytokinin and auxin in the basal MS medium was used to induce cloning. Low levels of both growth regulators typically range from 0.1 mg/L to 0.5 mg/L For sugar beet, MS salts, 30 g/L sucrose and appropriate gelling agent with 0.2 mg/L kinetin, and 0.1 mg/L NAA is used, Some months after inoculation, transgenic shoots were confirmed by means of the PMI-assay or PCR analysis, Clonal propagation and rooting of transgenic shoots were performed on standard propagation and rooting medium, under maintained mannose selection to eliminate chimeric plants that escaped the selection procedure. Finally plants were sent to greenhouse for phenotype testing.

Single shoots or clones are successfully rooted when transferred to a rooting medium containing MS basal medium supplemented with an auxin such as IBA at 0.5 mg/L to 5 mg/L. In one example, the rooting medium contains 5 mg/L IBA and about 12-15 g/L mannose.

It is understood that transformation of plant species is now routine for an impressive number of plant species, including both the Dicotyledoneae as well as the Monocotyledoneae. In principle any transformation method may be used to introduce chimeric DNA according to the invention into a suitable ancestor cell, as long as the cells are capable of being regenerated into whole plants. Methods may suitably be selected from the calcium/polyethylene glycol method for protoplasts (Krens, F. A. et al., 1982; Negrutiu I. et al, 1987), electroporation of protoplasts (Shillito R. D. et al., 1985), microinjection into plant material (Crossway A. et al., 1986), DNA or RNA-coated particle bombardment of various plant material (Klein T. M. et al., 1987), infection with (non-integrative) viruses and the like). One method according to the invention comprises Agrobacterium-mediated DNA transfer. Another method according to the invention is the use of the so-called binary vector technology as disclosed in EP A 120 516 and U.S. Pat. No. 4,940,838.

### Example 5

### Growth Conditions for T0 Generation Plants

Plasmid pHiNK260 was transformed in both annual and biennial sugar beet acceptor genotypes, while pHiNK382 was transformed in biennial material only. The first generation of transformed plants is called T0. Later generations are called T1, T2 etc. Seed was used for experiments using T1, T2 etc generations.

In order to create non-transgenic (NT) control plants, in vitro regenerated sugar beet shoots were produced. Besides of the actual transformation and selection procedures these NT shoots were treated and rooted similar to the transformed shoots. Every delivery of transgenic plants to the greenhouse was accompanied with at least one NT control plant of the same genotype.

After transfer to the greenhouse, the T0 transformed and NT control shoots were submitted to a rooting phase. The small plants were planted in small pots with soil and grown under enhanced CO₂ conditions for two weeks. After these two weeks, the rooted plants were transferred to 12 cm (0.7 liter) pots.

After the rooting phase, annual sugar beet plants were transferred to Biochamber KK3 (17 hours artificial light; 18° C. day+night temperature). The arrival day in KK3 was ‘Day 0’ and considered the start of the phenotypic analysis experiment.

After the rooting phase, biennial sugar beet plants were transferred to greenhouse VH113 (17 hours light, temperature 18-25° C. day and 15° C. night) for 2 weeks prior to vernalization. Vernalization occurred in cold room KK6 at a constant temperature of 6° C. and 12 hours under low artificial light for several weeks. Generally, sugar beet plants with the genetic background G018 were vernalized for 14 weeks, while G024 material Was vernalized for 16 weeks. The day that the plants were taken out of the vernalization room was ‘Day 0’ and considered the start of the phenotypic analysis experiment. Plants were first slowly acclimatized for two weeks in Biochamber KK5, stepwise increasing the temperature from 10 to 18° C., and subsequently repotted in larger, 16 cm (2 liter) pots and transferred to biochamber KK3.

The phenotypic analysis of the T0 generation events were started on a continuous basis and generally lasted for 3 months (90 days) or until all plants had started bolting. Plants which still had not started bolting after 3 months, were called Non-Bolting (NB) and were re-vernalized in an attempt to induce bolting and flowering for production of the next generation.

### Example 6

### Growth Conditions of T1, T2, T3 Generations

Summary of Growth Conditions: Phenotypic analysis experiments started from seed. Seed was germinated in 96-format plug-pot trays. In order to establish a uniform germination and root formation, the trays were grown at 17 hours light and temperatures of 18-25° C. day and 16° C. night. After two weeks, the plants were sampled for PCR analysis.

PCR analysis was carried out in order to identify the NT and transgenic plants in the progeny populations. This was achieved by means of, a POP reaction for either the transgene cassette or the selectable marker PMI. Populations segregating in annual and biennial plants were also tested with markers for the B-gene controlling the annual habit.

Using the PCR results, both transgene and NT plants were selected. NT plants functioned as internal control plants and accompanied the transgenic plants throughout the experiment. Only vigorous plants were selected and potted up for the phenotypic analysis of annual plants (Day 0). The biennial plants were kept in the plug-pots and artificially vernalized before entering the experiment. In the second semi-field trial described, biennial plants were planted out before vernalization.

Following the selection of the plants, the phenotypic analysis experiments employed different growth conditions as detailed hereinbelow:

Experiment 02-703 was carried out in greenhouse VH113 in 2002. The annual plants entered the phenotypic analysis directly upon PCR analysis, while the biennial plants were first vernalized in KK6 for 14 weeks. The procedure for vernalization and acclimatization in KK5 was identical as for the T0 generation.

Experiment 02-741 and 735 were combined and carried out in greenhouse VH113. Only biennial plants were selected and these were vernalized in KK6 for 17 and 19 weeks. After the 2 week acclimatization in KK5, vigorous plants were re-potted and transferred to the greenhouse VH113 in the first week of May, 2003.

Experiment 03-753 was carried out in greenhouse VH114. Vernalization occurred artificially in KK6 for 17 and 19 weeks and acclimatization for 2 weeks in KK5. Plants were transplanted in VH114 at the end of April and early May 2004. In this greenhouse, biennial plants were grown in the soil instead of pots. The experiment was therefore called a semi-field trial.

Experiment 04-754 and 755 were combined and carried out as a semi-field trial in greenhouse VH114 from September 2004 to May 2005. Vernalization occurred naturally in the unheated but frost-free greenhouse VH114. The plants were exposed to 13 weeks of mild vernalization (7-12° C.) and 15 weeks of strong vernalization (3-7° C.). Vigorous left-over plants were vernalized artificially for 18 weeks at 6° C. in KK6 and after two weeks of acclimatization in KK5 transferred to VH113 during the middle of March 2005.

Experiment 04-766 and 767 were combined and carried out in the climate chamber KK11 (16 hours light, temperature 18° C. day and 12° C. night). Vigorous biennial plants were vernalized in KK6 for 15 weeks at 6° C. and acclimatized in KK5 for 2 weeks before re-potting and transfer to KK11 (16 hours light, temperature 18° C. day and 12° C. night.

Bolting was scored up to three times per week during the phenotypic analysis experiments. The day of bolting was defined as the first day that stretching of the internodes of the meristem was first visible.

The above experiments are described now in more detail.

### Semi Field Trail VH114 September 2004-May 2005

### Experiment 04-754 AND 755

Seeds were germinated in a greenhouse with both natural and artificial light and heat, in order to obtain uniform germination. After two weeks, the plants in 96-format plug trays were transferred to more natural autumn conditions.

After six weeks, on 20 and 21 Oct. 2004, selected plants were planted out in VH114, in the soil with a conventional field trial layout. Temperature measurements were taken at canopy height (air) and at 10 cm soil depth (soil).

Drilling
Greenhouse VH113; week 37-39 (Mid September 2004)

| | |
|---|---|
| Temperature: | 18-25° C. day and 16° C. night |
| Light: | 17 H artificial + 12 H natural light; |
| | Metal halide lamp OSRAM Power Star HQI-BT |
| | 400W/D; >150-200 μmol/m² |
| Watering: | On daily basis |
| CO₂: | ambient |
| Pot size: | Plug pot trays (96-format tray, wells 4 × 4 cm) |

During daytime, light intensity could increase >800 μmol/m² due to sunlight. The lights were switched off when light intensity was >35 klux (600 μmol/m²)

Pre-vernalization
Greenhouse VH111; week 40-43 (End September-Mid October 2004)

| | |
|---|---|
| Temperature: | 10-15° C. |
| Light: | 12-10 H natural light |
| Watering: | On daily basis |
| CO₂: | Ambient |
| Pot size: | Plug pot trays (96-format trays, wells 4 × 4 cm) |

Night vernalization. Plants are transplanted before vernalization.

Vernalization
‘Natural’, 22 weeks
Greenhouse VH114; week 43-12
(Mid October 2004-End March 2005)

| | |
|---|---|
| Temperature air: | 0 < X < 12° C. until the end of March (week 13) at canopy height |
| Temperature soil: | 5 < X < 10° C. Nov-end March (week 13) at 10 cm below surface |
| Light: | Natural light and day length (10 H Oct - 6 H Dec - 12 H March) and |
| | minimized diffuse light from neighboring greenhouses. |
| | Below 200 total radiation PAR until first week February (week 5), |
| | increasing PAR up to 1200 PAR in Mid April (week 6-15) |
| Watering: | Seldom - only a few times in total, but like a heavy rain poor. |
| CO₂: | Ambient |
| Pot size: | Plants planted in soil as on field (18 cm in rows and 48 cm |
| | between rows) |
| Comments: | Temperature: one peak below 0° C. (4 Mar. 2005 at 05:18): −0.49° C. |

### Acclimatization

No special temperature acclimatization

Post-vernalization
Greenhouse VH114; week 12-19 (April-Mid May 2005)

| | |
|---|---|
| Temperature air: | Most day temp 15-25° C.; most night temp. |
| | 5-10° C. at canopy height |
| Temperature soil: | Most day temp 12-15° C.; most night temp. |
| | 7-10° C. 10 cm from surface |
| Light: | Natural light and day length 13-16 H >800 |
| | total radiation PAR on most days from early |
| | April |
| Watering: | Seldom - only a few times, but like a heavy |
| | rain shower. |
| CO₂: | Ambient |
| Pot size: | Plants planted in soil as on field (18 cm in |
| | rows and 48 cm between rows) |

### Greenhouse VH113 September 2004-May 2005

### Experiment 04-754 and 755

Seeds were germinated in a greenhouse with extra light and heat, in order to obtain uniform germination (same batch as for VH114 experiment). After two weeks; the plants in 96-format plug trays were transferred to more natural autumn conditions.

After six weeks, the left over plants from the semi field trial experiment were artificially vernalized for 18 weeks at 6° C. After artificial acclimatization in steps to 18° C., the plants were re-potted and transferred to a greenhouse with additional and natural light and heat. Air temperature measurements were taken 50 cm above the tables of the climate chamber and greenhouse.

Drilling
Greenhouse VH113; week 37-39 (Mid September 2004)

| | |
|---|---|
| Temperature: | 18-25° C. day and 16° C. night |
| Light: | 17 H artificial + 12 H natural light; |
| | Metal halide lamp OSRAM Power Star HQI-BT |
| | 400W/D; 150-200 1 μmol/m² |
| Watering: | On daily basis |
| CO₂: | ambient |
| Pot size: | Plug pot trays (96-format tray, wells 4 × 4 cm) |

During daytime, light intensity could increase >800 μmol/m² due to sunlight. The lights were switched off when light intensity was >35 klux (600 μmol/m²)

Pre-vernalizaiion
Greenhouse VH111; week 40-43 (End September-Mid October 2004)

| | |
|---|---|
| Temperature: | 10-15° C. |
| Light: | 12-10 H natural light |
| Watering: | On daily basis |
| CO₂: | Ambient |
| Pot size: | Plug pot trays (96-format trays, wells 4 × 4 cm) |
| Comments: | Night vernalization |

Vernalization
Artificial, 18 weeks
Climate chamber KK6; week 43-8
(Mid October 2004-End February 2005)

| | |
|---|---|
| Temperature: | Set at 6° C., temperatures 4-8° C. |
| Light: | 12 H artificial light, 8 H incandescent lamp, and 4 H |
| | metalhalide lamp |
| Watering: | Weekly basis |
| CO₂: | Ambient |
| Pot size: | Plug pot trays (96-format trays, wells 4 × 4 cm) |

During nights with sincere frost, temperatures could have been <6° C.; but >0

Acclimatization
Climate chamber KK5; Week 8-10 (Early March)

| | |
|---|---|
| Temperature: | [Day 10 + Night 8° C.] to [Day 18 + |
| | Night 12° C.] gradually during 14 days |
| Light: | 12 H artificial light; metal halide lamp |
| | 100 ± 30 μmol/m² |
| Watering: | Weekly basis |
| CO₂: | Ambient |
| Pot size: | Plug pot trays (96-format trays, wells 4 × 4 cm) |

Plants are transplanted at this stage after vernalization

Post-vernalization
Greenhouse VH113; week 10-19 (Mid March-Mid May 2005)

| | |
|---|---|
| Temperature: | 18-25° C. day and 15° C. night |
| Light: | 17 H artificial and 10-16 H natural light; |
| | metalhalide lamp OSRAM Power Star HQI-BT |
| | 400W/D >150-200 μmol/m² |
| Watering: | On daily basis |
| CO₂: | Ambient |
| Pot size: | 2 liter pots |

During daytime, light intensity could increase >800 μmol/m² due to sunlight. The lights were switched off when light intensity was >35 klux (600 μmol/m²)

### Climate Chamber KK11 (November 2004-June 20005)

### Experiment 04-766 and 767

Seeds were germinated in a greenhouse with extra light and heat, in order to obtain uniform germination. After three weeks, the plants in 96-format plug trays were artificially vernalized for 16 weeks at 6° C. After artificial acclimatization in steps to 18° C., the plants were re-potted and transferred to a climate chamber with artificial post-vernalization conditions and with close to ambient CO₂ levels at 400 ppm. Due to lack of space, the plants were potted up in 12 cm (0.7 litre) pots; smaller than in the VH113 experiment. Air temperature measurements were taken 130 cm above the tables and canopy height of the climate chamber.

Drilling
Greenhouse VH113; week 48 (End November 2004)

| | |
|---|---|
| Temperature: | 18-25° C. day and 16° C. night |
| Light: | 17 H artificial + 8 H natural light; |
| | Metal halide lamp OSRAM Power Star HQI-BT |
| | 400W/D; >150-200 μmol/m² |
| Watering: | On daily basis |
| CO₂: | Ambient |
| Pot size: | Plug pot trays (96-format trays, wells 4 × 4 cm) |

During daytime, light intensity could increase >800 μmol/m² due to sunlight. The lights were switched off when light intensity was >35 klux (600 μmol/m²)

Pre-vernalization
Greenhouse VH113; week 49-51, 2004)

| | |
|---|---|
| Temperature: | 18-25° C. day and 16° C. night |
| Light: | 17 H artificial + 8-6 H natural light; |
| | Metal halide lamp OSRAM Power Star HQI-BT |
| | 400W/D; 150-200 μmol/m² |
| Watering: | On daily basis |
| CO₂: | Ambient |
| Pot size: | Plug pot trays (96-format trays, wells 4 × 4 cm) |

Vernalization
Artificial, 16 weeks
Climate chamber KK12; week 51- 14
(End December 2004-Early April 2005)

| | |
|---|---|
| Temperature: | 5-7° C. |
| Light: | Artificial metalhalide lamp, 150 μmol/m2 day |
| | length 12 H?? |
| Watering: | On daily basis |
| CO₂: | Ambient |
| Pot size: | Plug pot trays (96-format trays, wells 4 × 4 cm) |
| Comments: | Nice vegetative growth, better than KK6; lighter |
| | than in KK6 |

Acclimatization
Climate chamber KK5; Week 14-16 (Mid April 2005)

| | |
|---|---|
| Temperature: | [Day 10 + Night 8° C.] to [Day 18 + |
| | night 12° C.] gradually during 14 days |
| Light: | 12 H artificial light; metal halide lamp |
| | 100 ± 30 μmol/m² |
| Watering: | On daily basis |
| CO₂: | Ambient |
| Pot size: | Plug pot trays (96-format trays, wells 4 × 4 cm) |

Plants are transplanted at this stage after vernalization.

Post-vernalization
Climate Chamber OK125: 11; week 16-24 (End April-Mid June 2005)

| | |
|---|---|
| Temperature: | 18° C. day and 12° C. night |
| Light: | 16 H artificial; Metal halide lamp, 200 μmol/m2 |
| Watering: | On daily basis |
| CO₂: | 400 ppm |
| Pot size: | 12 cm pots |
| Comments: | 15 plants/tray. Dense growth week 16-19 |
| | 9 plants/tray week 19-24. |

### Example 7

### Bolting Behavior of AGL20 (pHiNK382) and FLC (pHiNK260) Events

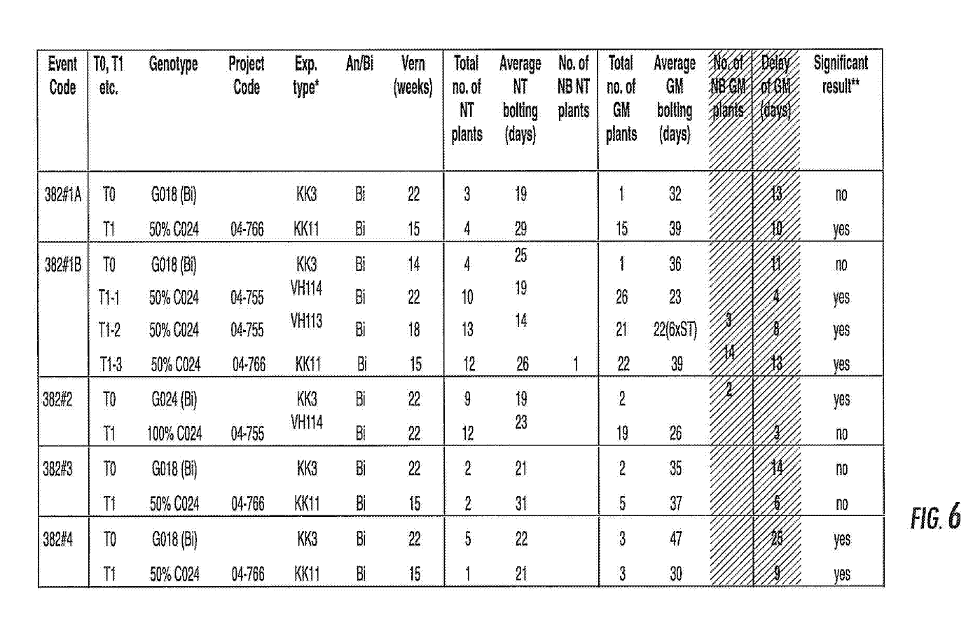

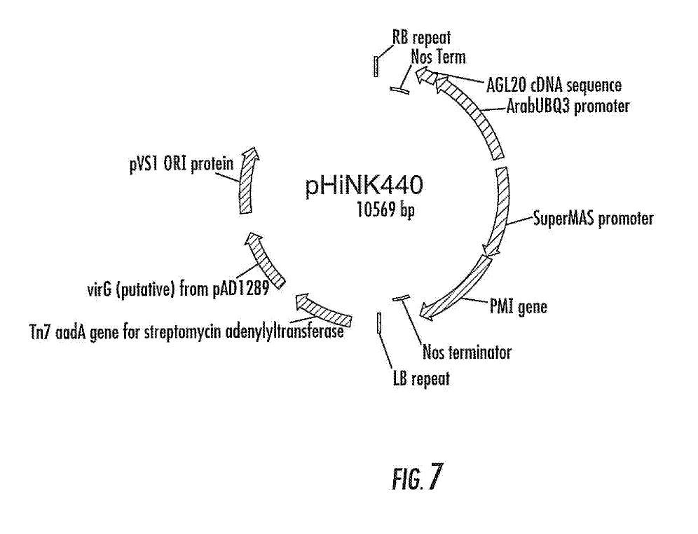

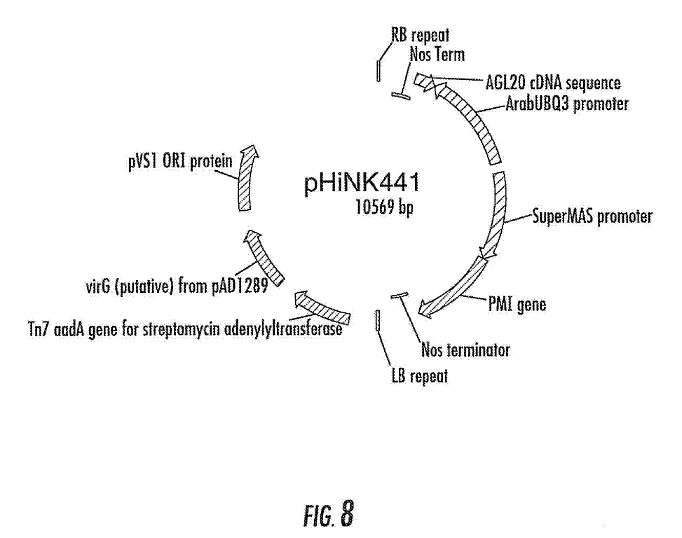

Out of 155 pHiNK260 events overexpressing the FLC gene 34 showed a delay in bolting either in an annual or a biennial background; out of 148 pHiNK382 events suppressing the endogenous AGL20 gene 22 showed a delay in bolting following following a typical vernalization treatment. The strongest events were forced to set seed and the progeny populations of 13 pHiNK260 and 21 pHiNK382 events were tested again for bolting resistance to confirm the results obtained for the T0 generation. The results of the four best pHiNK260 and pHiNK382 events are displayed in FIGS. 5 and 6, respectively, summarizing the results obtained in various generations and phenotypic experiments.

The average bolting day of the transgenic plants was always compared to the average bolting day of the NT control plants. The delay in bolting was calculated as the difference between these two averages (FIG. 5). For instance in experiment T3-04-755, the 24 NT plants of event 260#1 started bolting after 21 days on average. The 12 transgene 26041 plants started bolting after 61 days on average. The delay of bolting for this event in this experiment was therefore 61−21=40 days. In addition, Duncan grouping was carried out in order to test if the differences of NT and transgene bolting times were significant, which is indicated in the final column of FIGS. 5 and 6.

When the plants had still not started bolting at the end of the experiment, the result was recorded as NB (Non-Bolting). In some occasions, some plants did and others did not start bolting during the experiment. For instance in experiment T3-1 04-755, the 19 NT plants of event 260#1 started bolting after 20 days on average. Sixteen of the 17 transgene 260#1 plants started bolting after 61 days on average. One transgene plant, however, did not start bolting. The result of this event was therefore recorded as 17 plants; 61 & 1xNB. The delay of bolting for this event in this experiment was therefore 61−20=41 days & 1xNB.

Not surprisingly, different results were obtained when testing the events under different conditions in the different biochambers and greenhouses. For this reason, the bolting data of the transgenic plants were always compared to the bolting data of the NT control plants.

The climate chamber KK11 was the least bolting inductive. Extra delays in bolting were observed, also of NT plants. The low light intensity of 200 μmol/m2 was probably the limiting factor for rapid bolting induction in this climate chamber. Nevertheless, three out of 4 pHiNK382 events tested in KK11 displayed significant delays in bolting (FIG. 6).

The experiments conducted in greenhouse VH113 most frequently showed non bolting plants, notably for the pHiNK260 events #1, #2 and #3 (FIG. 5). For example out of 54 plants analyzed for the T2 generation of event pHiNK260 #1, none of the plants bolted (FIG. 5, experiment T2-1 02-741 and T2-2 02-735). Also pHiNK382 event #1B showed non-bolting plants in the T1 generation (FIG. 6, experiment T1-2 04-755, 3 out of 21).

The conditions of the semi-field trials in VH114 were the most bolting inductive. The soil was cold much longer compared to conditions in pots on tables in a greenhouse following artificial vernalization in cold rooms. Especially the semi-field trial over winter 2004-2005 was extremely bolting inductive. Plants in this semi-field trial perceived 22 weeks (5 months) of vernalizing temperatures, with a high number of accumulating cold degrees (average 5.2° C.). Despite the extreme long vernalization period, plants comprising a FLC or AGL20 event showed significant bolting delay.

### Example 8

### Bolting Control Under Highly Bolting Inductive Conditions

The following experiment which is described was carried out during the putative winter beet growing season of 2005-2006. Bolting control was further monitored in pHiNK260 and pHiNK382 events under highly bolting inductive conditions.

### 8.1 Plant Material

Entries consisted of T2 to T4 generations, which were created by crossing individual hemizygous transgenic plants of the selected events with non-transgenic plants. Therefore, each generation segregated in transgenic and non-transgene (NT) plants. The phenotypic screens always consisted of both classes of plants, which were handled and grown identically. In such way, the bolting behaviour of the transgenic plants could be studied and compared to NT plants in the same genetic background. Identification of the transgenic and NT plants in segregating progenies was carried out using PCR analysis as described before. Besides the pollinators used for research purposes, the best two AtFLC events #1 and #2B were also crossed with a potential commercial pollinator.

### 8.2 Growth Conditions

Geographic information System (GIS) temperature curves were used in order to come even closer to field conditions than in previous semi-field trials. Average weekly maximum, minimum and mean temperatures obtained over the last 12 years (1994-2005) were taken into consideration.

Vernalization in sugar beet occurs between 3 and 12° C. and the GIS data selected in Northern/Mid France are the one with the longest period with vernalizing temperatures on average in Europe. In such way, a bolting experiment was created under extreme stringent bolting conditions.

### 8.3 Summary of the Growth Conditions

Seeds were drilled and germinated in trays in 96-format plug-pot trays in biochamber KK14. In order to establish a uniform germination and root formation, the trays were grown at: 18 hours light and temperatures of 18-21° C. After 2 weeks, the plants were sampled for identification of the transgenic and NT plants by PCR. Stepwise, the temperatures of the biochamber were lowered before the 4 week old plants entered the vernalization period. Plants were transplanted directly into the soil of the greenhouse VH114. The temperature settings of this semi-field trial mimicked the average winter climate for Northern/Mid France: 4 weeks with average weekly temperatures between 0-3° C. and 25 weeks between 3-12° C. The trial was kept frost-free. In total the plants experienced 29 weeks of average weekly temperatures below 12° C. which is considered extremely bolting inductive. During spring the temperature increased slowly, so no special acclimatization period was introduced.

### 8.3.1 Detailed Growth Conditions

Drilling
Growth chamber KK14; week 39-41 (End September-Mid October 2005)

| | |
|---|---|
| Temperature: | 20-21° C. day and night (First 5 days) 18° C. day |
| | and night (after 5 days) |
| Light: | 18 H artificial (Metal halide lamp OSRAM Power Star |
| | HQI-BT 400W/D; >150-200 μmol/m2) |
| Watering: | On daily basis |
| CO2: | 800 ppm |
| Pot size: | Plug pot trays (96-format tray, wells 4 × 4 cm) |

Pre-vernalization
Growth chamber KK14; week 42-44 (Mid October-Early November 2005)

| | |
|---|---|
| Temperature: | 16° C. day and 8° C. night (gradual daily increase |
| | and decrease of temperature with night vernalization) |
| Light: | 12 H artificial (Metal halide lamp OSRAM Power Star |
| | HQI-BT 400W/D; >150-200 μmol/m2) |
| Watering: | On daily basis |
| CO2: | Ambient 400 to 800 ppm, depending of plant development |
| Pot size: | Plug pot trays (96-format trays, wells 4 × 4 cm) |

Vernalization
Greenhouse VH114; week 44-16 (Early November 2005-Mid April 2006)

| | |
|---|---|
| Temperature | French GIS data to follow. |
| air: | Nov. day 10° C./night 6° C. |
| | Dec. day and night 2-7° C. |
| | Jan. day and night 2-7° C. |
| | Feb. day 10° C./night 4° C. |
| | March/April day 12° C./night 4° C. |
| Light: | Natural light and day length (10 H Oct - 6 H Dec - |
| | 14 H April) |
| | Minimized diffuse light from neighboring greenhouses |
| | Below 200 total radiation PAR until first week Febr |
| | (week 5), increasing PAR up to 1200 PAR in End April |
| | (week 6-16) |
| Watering: | Seldom - only a few times in total, but like a heavy |
| | rain poor. |
| CO2: | Ambient |
| Pot size: | No pots: Plants planted directly in soil (20 cm in rows |
| | and 50 cm between rows). |

Post-vernalization
Greenhouse VH114; week 16-24 (April-Mid June 2006)

| | |
|---|---|
| Temperature | Min. day temp 15° C.; Min. night temp. 8° C. |
| air: | at canopy height |
| Light: | Natural light and day length 14-18 H >800 total |
| | radiation PAR on most days from early April |
| Watering: | On weekly basis |
| CO2: | Ambient |
| Pot size: | No pots: Plants planted directly in soil (20 cm |
| | in rows and 50 cm between rows) |

### 8.4 Results and Discussion

TABLE 1
Phenotypic results of the selected pHiNK260 (FLC) and pHiNK382
(AGL20) events in the semi-field trial 2005-2006

| | | Total no. | Average NT | No. of | Average GM | Delay | Delay GM |
|---|---|---|---|---|---|---|---|
| | Event | of NT | bolting | bolting | bolting | of GM | Significant |
| | Code | plants | (days) | GW plants | (days) | (days) | (Duncan) |
| Semi-field | FLC#1 | 12 | 15 | 24 | 46 | 31 | Yes |
| trial | FLC#1 | 11 | 6 | 24 | 34 | 28 | Yes |
| ’French’ | hybrid | | | | | | |
| winter | FLC#2B | 12 | 16 | 24 | 38 | 22 | Yes |
| Followed by | FLC#2B | 12 | 5 | 24 | 31 | 26 | Yes |
| ’French’ | hybrid | | | | | | |
| spring | FLC#2AB | 48* | 12* | 24 | 24 | 12 | Yes |
| | FLC#4 | 12 | 12 | 24 | 33 | 21 | Yes |
| | FLC#5 | 12 | 11 | 24 | 20 | 9 | Yes |
| | AGL20 | 12 | 14 | 24 | 20 | 6 | Yes |
| | 382#1 | | | | | | |
| | AGL20 | 12 | 18 | 24 | 18 | 0 | No |
| | 382#2 | | | | | | |
| | AGL20 | 12 | 15 | 24 | 21 | 6 | Yes |
| | 382#3 | | | | | | |
| | AGL20 | 12 | 12 | 24 | 17 | 5 | Yes |
| | 382#4 | | | | | | |

Bolting was scored and defined as the first visible elongation of the apical meristem. Day 1 was 30 Mar. 2006, the first day that bolting in the NT controls was detected. Scoring for bolting was stopped after 12 weeks on day 84. The significance of the gene effect was assessed by applying the statistical Duncan's Multiple Range Test
*Data from reference NT plants of other entries with the same genetic background.
NT Non-transgenic control plants
GM Transgenic plants, transformed with either FLC plasmid pHiNK260 or AGL20 plasmid pHiNK382

All plants survived the winter conditions of the semi-field trial and were very vigorous in March. Sunny weather in March made the conditions for bolting favourable, and the first bolting non-transgene (NT) plant was detected on March 30. This day was the first day of counting bolting time. The fact that the conditions of the semi-field trial have been highly bolting inductive is demonstrated by the observation that not one single plant remained non-bolting throughout the experiment (84 days of counting). Even plants of the best pHiNK260 events which were non-bolting in previous experiments, eventually bolted.

All FLC entries were significantly delayed compared to the internal NT controls. The least delayed event was FLC 260 #5 that showed a delay of 9 days; the best events were FLC 260 #1 and #2B showing delays of 31 respectively 22 days. These two FLC events were also crossed with a commercial pollinator in order to study the bolting behaviour in a hybrid background. The hybrids were more vigorous than the research genotypes, and bolting was induced earlier in the NT hybrid controls. Nevertheless, the transgenic hybrids still showed a similar delay in bolting of 28 and 26 days respectively.

Also 3 out of 4 AGL20 events (pHiNK382) were significantly delayed in belting under these extreme stringent bolting conditions, albeit not to the same extend as the FLC events (6 days maximum).

### Example 9

### RNAi Hybrid Concept

### 9.1 Plant Material

The vectors pHiNK440 and 441 express only one strand of the BvAGL20 dsRNA fragment, sense and antisense orientations respectively, as is described in Example 3. The dsRNA for BvAGL20 is therefore obtained in the hybrid only, after crossing events for either vector to each other.

In order to test this RNAi hybrid concept; pHiNK440 was transformed into a female (male sterile) sugar beet line, while pHiNK441 was transformed into a sugar beet pollinator line. T0 events obtained were tested for the expression of the transgenes by RT-PCR and pHiNK440×pHiNK441 combined by crossing. The T1 populations segregated in 4 classes: 1) NT, 2) pHiNK440 only, 3) pHiNK441 only, and 4) the hybrid, pHiNK440×441. All 4 classes were identified by PCR and the expression of the transgenes studied by RT-PCR.

### 9.2 Materials and Methods

DNA was isolated using the GenElute Plant Genomic DNA Miniprep kit from Sigma. RNA was isolated using the RNAqueous-4PCR kit from Ambion. RNA was treated with Dnase I, and the Dnase then removed prior to cDNA production. RNA concentration was measured using the spectrofotometer.

cDNA was produced using the Omniscript Reverse Transcriptase kit and HotStart Taq-polymerase from Qiagen. 1 μg total RNA was used for each reaction and the oligo-dT primer at a total volume 20 μl. After the reverse transcription, the cDNA samples were diluted to 40 μl and used for the RT-PCR reaction at three different concentrations (0.5, 1.0 and 2.0 μl).

The (RT-)PCR set up was carried out in such a way that DNA or cDNA aliquots of all plants were identical for each PCR reaction. PCR reaction bulks were created, so that all plants would be tested with identical PCR mix.

Plasmid pHiNK 440 was identified using the primer pair AGL20 A (5′ GTC TCG AAC TTT CTA AAC GGA) and nos terminator primer HiNK023 (5′ CGC AAG ACC GGC AAC AGG ATT C). Plasmid pHiNK 441 was identified using the primer pair AGL20 B (5′ GAT CAT CTG CTC GTT GTT GG) and primer HiNK023.

As RNA household and internal positive control gene, the gene GAPC, Cytosolic glyceraldehyde-3-phosphate dehydrogenase, was used (Reeves et al, 2006) with the primer pair gapCex5/6F (5′ GCTGCTGCTCACTTGAAGGGTGG) and gapCex8R (5′ CTTCCACCTCTCCAGTCCTT).

Above three PCR reaction were carried out using a PCR programme with a hot start of 15 min at 9500, followed by 35 cycles of denaturing of 30 sec at 94°; annealing of 30 sec at 55° C. and a extension step for 30 sec (+2 sec/cycle) at: 72° C. The PCR was finished with a 5 min step at 72° C.

The endogenous BvAGL20 gene was amplified with BvAGL20 specific primers HiNK 729 (5′ AAG GTA GCA GAT CTC GTG AAG AAT TGA G) and HiNK 819 (5′ TCT GCG TGG AGT GAA AAG TAA AGT G) which cover the gene from putative exon 3 to 8. The PCR programme for BvAGL20 consisted of a hot start of 15 min at 95° C., followed by 30 cycles of denaturing of 30 sec; at 92°; annealing of 30 sec at 57° C. and a extension step for 2 min at 72° C. The PCR was finished with a 5 nm step at 72° C.

The PCR fragments were run on an electrophoresis gel with a composition of different samples of one plant per lane: 1) Water: Negative, no amplification control in order to test if the PCR mix was contaminated. 2) DNA (50 ng/reaction): Positive control of plant DNA in order to confirm that the plant is transgene 3) RNA (200 ng/reaction): Negative, no RT-PCR control in order to check if DNA was successfully removed, and 4) cDNA (0.5, 1.0 and 2.0 μl): Test samples which represents the RNA and should give expression levels.

### 9.3 Results

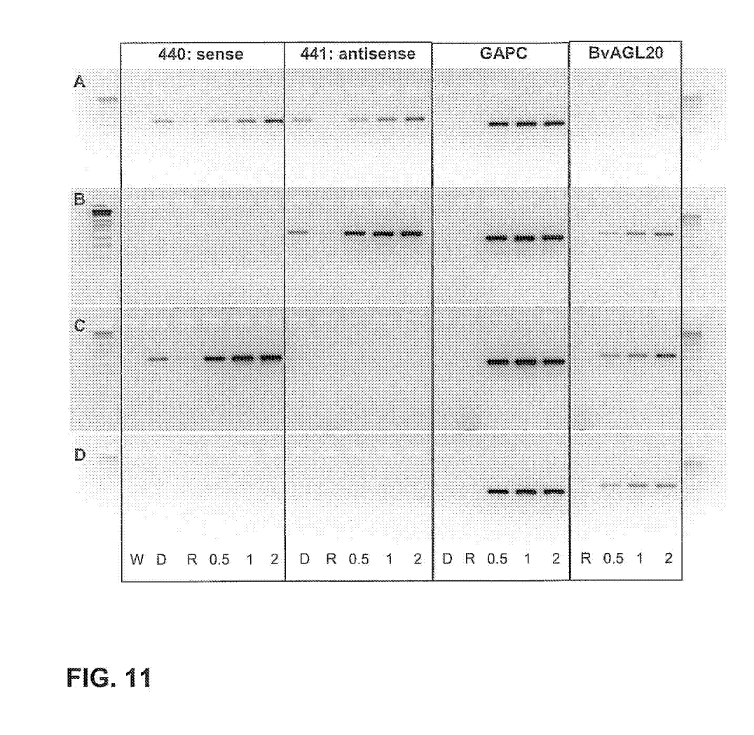

This example describes the hybrid of a cross between a parental pHiNK440 line and a pHiNK441 line with high expression for the transgenes. The RT-PCR results of 4 plants of the progeny, one of each class, are shown in FIG. 11. The results show that the endogenous BvAGL20 gene was down regulated in the hybrid only, but not in the NT, nor plants with a single dsRNA component.

### Example 10

### Stacked Hybrids of FLC (pHiNK260) and AGL20 (pHiNK382) Events

### 10.1 Plant Material

In addition to monitoring the bolting behaviour of FLC and AGL20 events individually, a limited number of stacked hybrids between both types of events were produced. Crosses between individual plants of FLC and AGL20 events resulted in a segregating population segregating in four different classes: 1) FLC alone, 2) AGL20 alone, 3) Stacked hybrid FLC & AGL20 or 4) NT. All young plants were screened by PCR for their identity, and all four classes entered the phenotypic screen.

### 10.2 Growth Conditions

Plants of these segregating stacked hybrid populations were artificially vernalized in a biochamber for 16 weeks. Mid April, the plants entered the semi-field trial experiment described in Example 8. Plants of all four classes were transplanted directly into the soil of greenhouse VH114.

### 10.3 Detailed Growth Conditions of the Stacked Hybrids

Drilling
Growth chamber BK6; week 50-52
(Mid December 2005-early January 2006)

| | |
|---|---|
| Temperature: | 20-21° C. day and night (First 5 days) 18° C. day |
| | and night (after 5 days) |
| Light: | 18 H artificial (Metal halide lamp OSRAM Power Star |
| | HQI-BT 400W/D; >150-200 μmol/m2) |
| Watering: | On daily basis |
| CO2: | 800 ppm |
| Pot size: | Plug pot trays (96-format tray, wells 4 × 4 cm) |

Pre-vernalization
Growth chamber BK6; week 1-3 (Early January 2006)

| | |
|---|---|
| Temperature: | 16° C. day and 8° C. night (gradual daily increase |
| | and decrease of temperature with night vernalization) |
| Light: | 12 H artificial (Metal halide lamp OSRAM Power Star |
| | HQI-BT 400W/D; >150-200 μmol/m2) |
| Watering: | On daily basis |
| CO2: | Ambient 400 to 800 ppm, depending of plant development |
| Pot size: | Plug pot trays (96-format trays, wells 4 × 4 cm) |

Vernalisation
Growth Chamber KK12; week 1-16 (Early January-Mid April 2006)

| | |
|---|---|
| Temperature | 5-7° C. |
| air: | |
| Light: | Artificial light 12 H (Metal halide lamp OSRAM |
| | Power Star HQI-BT 400W/D; >150-200 μmol/m2) |
| Watering: | On daily basis |
| CO2: | Ambient |
| Pot size: | Plug pot trays (96-format trays, wells 4 × 4 cm) |

### Post-Vernalization

### Greenhouse VH114, Week 16-24 (April-Mid June 2006)

The plants entered the experiment described in example 8. The first day of counting bolting time was the day that the plants left the vernalization biochamber.

### 10.4 Results and Discussion

Table 2: Phenotypic results of stacked hybrids after artificial vernalization. Bolting was scored and defined as the first visible elongation of the apical meristem. Day 1 was the day that the plants left the artificial vernalization and entered the semi-field trial of Example 8. Scoring for bolting was stopped after 98 days. The significance of the gene effect was assessed by applying the statistical Duncan's Multiple Range Test.

TABLE 2

| | | Segregating population of cross AGL20 × FLC | | | | | | | |
|---|---|---|---|---|---|---|---|---|---|
| | | NT | | AGL20 | | | FLC | | |
| Parental components | | | Average | | Average | Delay in | | Average | Delay in |
| AGL20 | FLC | No. of | Bolting | No. of | Bolting | bolting vs | No. of | Bolting | bolting vs |
| event | events | plants | (days) | plants | (days) | NT (days) | plants | (days) | NT (days) |
| 382#1 | 260#2A | 4 | 16 | 14 | 23 | 7 | 2 | 26 | 10 |
| 382#1 | 260#2B | 8 | 15 | 4 | 45 | 30 | 2 | 40 | 25 |
| | | | | | | | 1 | — | NB |
| 382#1 | 260#4 | 30* | 17* | 8* | 16* | −1 | 24 | 36 | 19 |
| 382#1 | 260#6 | 5 | 14 | 3 | 19 | 5 | 9 | 29 | 15 |

| | | Segregating population of | | | | |
|---|---|---|---|---|---|---|
| | | cross AGL20 × FLC | | | | |
| | | Stacked hybrid | | | Significant Delay | Additive/ |
| Parental components | | | Average | Delay in | Stack vs single | Synergistic |
| AGL20 | FLC | No. of | Bolting | bolting vs | AGL20 or FLC | gene effects in |
| event | events | plants | (days) | NT (days) | plants (Duncan) | stacked hybrid |
| 382#1 | 260#2A | 10 | 40 | 24 | Yes | Yes |
| | | 6 | — | NB | | |
| 382#1 | 260#2B | 2 | — | NB | Yes | Yes |
| 382#1 | 260#4 | 9 | 47 | 30 | Yes | Yes |
| | | 15 | — | NB | | |
| 382#1 | 260#6 | 4 | — | NB | Yes | Yes |

*Data from reference NT plants of other entries with the same genetic background in the same experiment.
NT Non-transgene plants
NB Non-bolting plants: Plants which did not show any signal of bolting throughout the whole experiment of 98 days

Stacked hybrids of AGL20 event pHiNK382#1 and 4 different FLC events showed a synergistic interaction of the FLC and AGL20 effects on bolting control (Table 2). For example, the effects of the individual events for the first combination was 7 days delay for AGL20 event 382#1 and 10 days for FLC event 260#1, which theoretically adds up to a delay of 17 days for the hybrid. The stacked hybrid, however, showed an additional delay in bolting: 24 days instead of 17 days. Moreover, 6 hybrid plants stayed non-bolting (NB) throughout the experiment for 98 days whereas non-bolting plants were not observed for neither of the individual events. Similar synergistic effects were also obtained for the other 3 hybrid combinations. Notably, while only one single plant of all FLC events analyzed stayed non-bolting, the majority of the stacked hybrids did not start bolting after 3 months, thus illustrating that combining events for the two bolting control genes created a highly significant and synergistic delay in bolting.

### Example 11

### Industrial Applications

The present invention further includes a method of deriving ethanol and/or sugar from the sugar beet plant of the present invention, wherein the root of the sugar beet plant is the predominant source of ethanol and/or sugar. The sugar and the ethanol derivable from the sugar beet plant and root of the sugar beet plant of the invention also fall within the scope of the present invention. Methods of extracted sugar and ethanol from sources such as sugar beet are very well known in the industry.

In summary, ethanol production includes first washing and then slicing the sugar beets followed by an extraction step. The extraction step produces two products: extracted sugar juice and the beet slices. The beet pulp is typically tried and pelletized and sold as as animal feed. Thus, beet pulp and animal feed derived from the sugar beet plant and root of the invention are within the scope of the present invention. The sucrose fraction is typically washed, sterilized or otherwise treated to prevent microbial contamination. The sucrose fraction is then fermented. There are numerous fermentation methodologies known to those skilled in the art. In one embodiment and by way of example only, Saccharomyces cerevisiae is the organism that is used in the fermentation step. During the fermentation a large amount of CO₂ is produced. The CO₂ is used to manufacture beverages, fire extinguishers and in food processing. The product of fermentation, with an alcohol content of 8-15% by volume is passed on to the distillation unit, where it is concentrated to 95%. A final dehydration step is required to remove the remaining water from the ethanol. Ethanol production is well known in the industry and various different methodologies can be used to produce the final ethanol fuel. Ethanol can also be produced by fermentation of sugar beet molasses, sugar juice, dry sugar beet powder and sugar.

Biogas can also be produced from sugar beet using method commonly known in the industry. Biogas consists of methane, carbon dioxide and a small amount of H₂S and ammonia and is produced during anaerobic fermentation of organic material. The fermentation process takes approximately 1 month. In most cases, the biogas is used for combined heat and power generation. The gas is burnt directly and produces heat that can be used for heating houses or generating power. It also can be used as fuel for vehicles.

Biodiesel can also be generated from sugar beet. Using Fischer-Tropsch synthesis, biogas can be converted to liquid fuel, FT-diesel. At present, the production from biomass is only at the pilot stage, and large-scale Fisher-Tropsch conversion installations using fossil fuels exclusively, most commonly natural gas. The advantage of FT-diesel is that its composition can be optimized for the combustion behavior of the motor. The fuel is free from sulfur and aromatic compounds and compared to ordinary diesel, the emissions contain 8% less nitrogen oxides, 30% less particulate matters, 30% less hydrocarbons (HC), 75% less carbon monoxide and 90% less polluting compounds.

### REFERENCES

Levy, Y. Y. and Dean, C. 1998. The Transition to Flowering. Plant Cell, 10:1973-1990
Koornneef, M., Blankestijn-de Vries. H. Hanhart, C., Soppe, W. and Peeters, T. 1994. The phenotype of some late-flowering mutants is enhanced by a locus on chromosome 5 that is not effective in the Landsberg erecta wild-type. The Plant Journal 6: 911-919.
Lee, I., Michaels, S. D., Masshardt, A. S. and Amasino, R. M. (1994) The late-flowering phenotype of FRIGIDA and mutations in LUMINIDEPENDENS is suppressed in the Landsberg erecta strain of Arabidopsis. Plant J. 6, 903-909.
Eckes P, Rosahl S, Schell J and Willmitzer L, 1986. Isolation and characterization of a light-inducible, organ-specific gene from potato and analysis of its expression after tagging and transfer into tobacco and potato shoots. Mol. Gen. Genet. 205, 14-22
Nilsson, O., Lee, I., Blázquez, M. A. and Weigel, D. (1998) Flowering-time genes modulate the response to LEAFY activity. Genetics, 150, 403-410.
Kobayashi, Y., Kaya, H., Goto, K., Iwabuchi, M. and Araki, T. (1999) A pair of related genes with antagonistic roles in mediating flowering signals. Science, 286, 1960-1962.
Lee, H., Suh, S.-S., Park, E., Cho, F., Ahn, J. H., Kim, S.-G., Lee, J. S., Kwon, Y. M. and Lee, I. (2000) The AGAMOUS-LIKE 20 MADS domain protein integrates floral inductive pathways in Arabidopsis. Genes Dev. 14, 2366-2376.
Samach, A. Onouchi, H., Gold, S. E., Ditta, G. S., Schwarz-Sommer, Z. Yanofsky, M. F. and Coupland, G. (2000) Distinct roles of CONSTANS target genes in reproductive development of Arabidopsis. Science, 288, 1613-1618.
Borner, R., Kampmann, S., Chandler. J, Gleissner, R., Wisman, E., Apel, A. and Melzer, S. (2000) A MADS domain gene involved in the transition to flowering in Arabidopsis. Plant J. 24, 591-599.
Elbashier et al. 2001, RNA interference is mediated by 21 and 22 nucleotide RNAs, Genes Dev. 15, 188).
Altschul et al., 1990, Basic local alignment search tool, J. Mol. Biol. 215: 403-410.
Joersbo M, Donaldson I, Kreiberg J, Petersen S G, Brunstedt J and Okkels F T, 1998. Analysis of mannose selection used for transformation of sugar beet. Mol. Breeding 4: 111-117.
Micheals, S D and R M Amasino, 1999. FLOWERING LOCUS C encodes a novel MADS domain protein that acts as a repressor of flowering. The Plant Cell 11: 949-956.
Hood E E, G L Helmer, R T Fraley and M-D Chilton, 1986. The hypervirulence of Agrobacterium tumefaciens A281 is encoded in a region of pTiBo542 outside of T-DNA. J Bacteriology 168: 1291-1301.
Higuchi, R, 1990. Recombinant PCR. In: PCR protocols, A guide to methods and application. Innis M A, Gelfand D H, Sninsky J J and White T J (Eds.), Academic Press Inc., San Diego. pp. 177-183.
Vancanneyt G, Schmidt R, O'Connor-Sanchez A, Willmitzer L and Rocha-Sosa M, 1990. Construction of an intron-containing marker gene: Splicing of the intron in transgenic plants and its use in monitoring early events in Agrobacterium-mediated plant transformations. Mol. Gen. Genet. 220: 245-250.
Wang M B and Waterhouse P M, 2001. Application of gene silencing in plants. Curr. Opin. Plant Biol. 5:124-150.
Norris S R, S E Meyer and J Callis, 1993. The intron of Arabidopsis thaliana polyubiquitin genes is conserved in location and is a quantitative determinant of chimeric gene expression. Plant Mol Biol 21: 895-906.
Holsters M, D de Waele, A Depicker, E Messens, M van Montagu and J Schell, 1978. Transfection and transformation of Agrobacterium tumefaciens. Mol Gen Genetics 163: 181-187.
Krens, F. A. et al., 1982, In vitro transformation of plant protoplasts with Ti-plasmid DNA, Nature 296, 72-74.
Negrutiu I. et al, 1987, Hybrid genes in the analysis of transformation conditions, Plant Mol. Biol. 8, 363-373.
Shillito R. D. et al., 1985, High efficiency direct gene transfer to plants, Bio/Technol. 3, 1099-1102.
Crossway A. et al., 1986, Integration of foreign DNA following microinjection of tobacco mesophyll protoplasts, Mol. Gen. Genet. 202, 179-185.
Klein T. M. et al., 1987, High-velocity microprojectiles for delivering nucleic acids into living cells, Nature 327, 70.
Smith N A, Singh S P, Wang M B, Stoutjesdijk P A Green A G and Waterhouse P M, 2000. Total silencing by intron-spliced hairpin RNAs. Nature 407: 319-320
Jaggard, K. W., Wickens, R., Webb, D. J. and Scott, R. K. 1983. Effects of sowing date on plant establishment and bolting and the influence of these factors on yields of sugar beet. Journal of Agricultural Science, Cambridge. 101, 147-161.
Needleman & Wunsch, 1970, A general method applicable to the search for similarities in the amino acid sequence of two proteins, J. Mol. Biol. 48: 443.
Pearson & Lipman, Proc. Nat'l. Acad. Sci. USA 85: 2444 (1988)
Tijssen (1993) Laboratory Techniques in Biochemistry and Molecular Biology—Hybridization with Nucleic Acid Probes part I chapter 2 “Overview of principles of hybridization and the strategy of nucleic acid probe assays” Elsevier, N.Y.

Pa\check{r}enicoavá L et al., 2003. Molecular and phylogenetic analyses of the complete MADS-box transcription factor family in Arabidopsis: New openings to the MADS world. Plant Cell 15: 1538-1551.
Reeves, Patrick A., He, Yuehui, Schmitz, Robert J., Amasino, Richard M., Panella, Lee W. and Richards, Christopher M. 2006 Evolutionary conservation of the FLC-mediated vernalization response; evidence from the sugar beet (Beta vulgaris) Genetics: Published Articles Ahead of Print, published on Dec. 18, 2006 as 10.1534/genetics.106.069336

1-77. (canceled)

78. A transgenic sugar beet plant comprising in its genome the coding region of a heterologous FLC gene, wherein expression of said FLC gene causes over expression of the FLC gene product thereby suppressing the vernalization response of said sugar beet plant.

79. The transgenic sugar beet plant according to claim 78, wherein said heterologous FLC gene comprises the FLC coding region consisting of the FLC cDNA of Accession No. AF537203.

80. The transgenic sugar beet plant according to claim 78, wherein said FLC gene is depicted by SEQ ID NO: 3.

81. The transgenic sugar beet plant according to claim 78 comprising an endogenous FLC gene as depicted in SEQ ID NOs: 21, 23 and 25 or the encoding part thereof.

82. The transgenic sugar beet plant according to claim 78, wherein said heterologous FLC gene comprises the FLC coding region which has at least between 93% and 99% sequence identity with the nucleotide sequence of the FLC cDNA of Accession No. AF537203.

83. The transgenic sugar beet plant according to claim 78, wherein said heterologous FLC gene has at least between 93% and 99% sequence identity with the nucleotide sequence depicted by SEQ ID NO: 3.

84. The transgenic sugar beet plant according to claim 78, wherein said endogenous FLC gene has at least between 93% and 99% sequence identity with the nucleotide sequence depicted in SEQ ID NOs: 21, 23 and 25 or with the encoding part thereof.

85. The transgenic sugar beet plant according to claim 78, wherein said heterologous FLC gene is incorporated in an expression cassette under the control of the constitutive promoter, particularly a CaMV 35S promoter, and a terminator, particularly the mannopine synthase (mas) terminator from Agrobacterium tumefaciens.

86. The transgenic sugar beet plant according to claim 78, wherein said heterologous or endogenous FLC gene is incorporated in an expression cassette depicted by nucleotide sequence 786-2817 of SEQ ID NO: 1.

87. A method of producing a transgenic sugar beet plant according to claim 78, comprising:
a) transforming a sugar beet plant cell with a expression cassette comprising a heterologous or an endogenous FLC gene, wherein said FLC gene is operably linked to regulatory sequences;
b) identifying a sugar beet plant cell carrying the transgene; and
c) regenerating a transgenic plant from said plant cell identified in b) of this claim.

88. The method of producing a transgenic sugar beet plant according to claim 87, wherein said FLC gene is depicted by SEQ ID NO: 3.

89. The method of producing a transgenic sugar beet plant according to claim 87, wherein said FLC gene has at least between 93% and 98% sequence identity with the nucleotide sequence depicted by SEQ ID NO: 3.

90. The method of producing a transgenic sugar beet plant according to claim 87, wherein said FLC gene is depicted in SEQ ID NOs: 21, 23 and 25.

91. The method of producing a transgenic sugar beet plant according to claim 87, wherein said FLC gene has at least between 93% and 98% sequence identity with the nucleotide sequence depicted SEQ ID NOs: 21, 23 and 25 or the encoding part thereof.

92. A method of producing a transgenic sugar beet plant according to claim 88, wherein said transgene is incorporated into an expression cassette depicted by nucleotide sequence 786-2817 of SEQ ID NO: 1.

93. A method of producing a transgenic sugar beet plant according to claim 92, wherein said transgene in said expression cassette is operatively linked to a constitutive promoter.

94. A method of producing a transgenic sugar beet plant according to claim 92, wherein said transgene in said expression cassette is operatively linked to a CaMV35S promoter.

95. A root of the transgenic sugar beet plant of claim 78, which root still exhibits the invention-relevant features of said sugar beet plant as defined in claim 78.

96. A progeny plant of a sugar beet plant of claim 78, wherein said progeny plant still exhibits the invention-relevant features of said sugar beet plant as defined in claim 78.

97. A seed of the transgenic sugar beet plant of claim 78, wherein said seed still exhibits the invention-relevant features of said sugar beet plant as defined in claim 78.

98. A method of producing a transgenic sugar beet plant according to claim 87, further comprising the steps of:
d) identifying a sugar beet plant exhibiting a delay of the vernalization response or a complete suppression of the vernalization response resulting in a non bolting (NB) phenotype; and
e) optionally confirming the presence of the heterologous DNA in the plant cell genome introduced in step a).

99. A method of producing sugar comprising processing a sugar beet plant according to claim 78 and deriving sugar from said sugar beet plant.

100. A method of producing ethanol comprising processing a sugar beet plant according to claim 78 and deriving ethanol from the sugar beet plant.

101. A method of producing biogas comprising processing a sugar beet plant according to claim 78 and deriving biogas from the sugar beet plant.

102. A method of producing diesel fuel comprising processing a sugar beet plant according to claim 78 and deriving diesel fuel from the sugar beet plant.
